SEQUENCE LISTING (48 sequences)

SEQ ID NO: 1 - protein, 435 aa, Artificial Sequence
  HMMer software generated consensus sequence
GGLRELMAGQLAVWHAQQLAPENPVYNVGEYVEIDGEVDLDLLVAAVRRVMEEADAARLRFREVDGVPRQYFAEDEDYPVEVIDVSAEADPRAAAESLMAADLRRPRDLRDGELYTQKIYKVGEDLVFWYQRAHHIILDGRSAGLVASRVAAVYSALAAGGDVEEGALPSSSVLMDAEDEYRASEEFELDREYWREALAGLPEEVSLGANEPSRLPREPVRHEEDVSDAAAAELRAAARRLGTSLAGLAIAAAALYQHRLTGQRDVVVGVPVAGRSKTAELDIPGMTANVVPVRLAVAPKTTVAELVRQVARGVRDGLRHQRYRYEDILDDLKLVGRDGLYPLLVNVLSFDYDLRFGDAVSVAHGLSAGPVDDVSIDVYDRSSDGSMKVVVDVNPDLTDRSDADEVARKFLALLRWLAESDAEEPVARIDLLDED

SEQ ID NO: 2 - protein, 451 aa, Artificial Sequence
  HMMer software generated consensus sequence
SVRHGVTAAQRGVWVAQQLRPDSRLYNCGLYLELDGALDPAVLSRAVRRTLAETEALRSRFEEDDDGALLQRVLAPAPDEQTRLLEDGVPYTPVLLRHIDLSGDDDPEAAARRWMDADLAEPVDLDRAGTSRHALLTLGGDRHLLYLGYHHIALDGFGAALYLDRLAAVYRALRTGREPPPCPFGPLDRLVAEEAAYRDSARHRRDRAYWTGRFADLPEPVGLAGRAAAAAPAPLRRTVRLPPERTAALAAAAEATGSRWPAVVIAAVAAFLRRLAGAEEVVVGLPVTARVTRAALRTPGMLANVLPLRLEVRQGASFAALLEETSRALSALLRHQRFRGEDLGRELGLAGERAGLAPTTVNVMAFAPVLDFGDCRAVVHQLSSGPVEDLAINLYGTPGTGDELRVTVAANPALYTADDVASLQERLVRFLAALGADPAAPVGRVRLLDPA

SEQ ID NO: 3 - protein, 603 aa, Artificial Sequence
  HMMer software generated consensus sequence
VSAVMVDLAAGPSVPAALRAHAEARPDRTAVVFVRDTDRADGTASLSYAELDRRARAVAVWLRARLAPGDRVLLLHPAGPEFVAAYLGCLYAGLVAVPAPLPGGYSHERRRVVGIAADAGAGAVLTDADTEAEVREWLAETGLPGLPVLAVDPLAADGDPGAWRPPGLRADTVAVLQYTSGSTGSPKGVVVTHGNLLANARSLSRSFGLTEDTVFGGWLPLYHDMGLFGLLLPALFLGATVVLMSPSAFLRRPHLWLRLIDRFGVVFSAAPDFAYDLCVRRVTDEQIAGLDLSRWRWAANGSEPIRAATLRAFAERFAPAGLRPEALTPCYGLAEATLFVSGKSAGPLRTRRVDPAALEDHRFEEAVPGRPAREIVSCGRVPDLEVRIVDPGTGRPLPDGAVGEIWLRGPSVAAGYWGRPEATAETFGAVTDGGDGPWLRTGDLGALYEGELYVTGRIKELLIVHGRNLYPHDIEHELRAAHDELAGAVGAAFAVPAPGGGEEVLVVVHEVRPRVPADELPALASAMRATVAREFGVPAAGVVLVRRGTVRRTTSGKVQRRAMRELFLTGELAPVHAELGPHLQAAAAGEARAATSLAPASTV

SEQ ID NO: 4 - protein, 91 aa, Artificial Sequence
  HMMer software generated consensus sequence
MSDLTAPPARHTPEELRAWLRECVADYVGLPPAEIATDVPLTDYGLDSVYALALCAEIEDHLGIEVDPTLLWDHPTIDELSAALAPRLARR

SEQ ID NO: 5 - DNA, 1305 bp, Actinoplanes sp.
cctgacctgcgcccgctcacgcccgcccagctcgccgtctggcacgcgcagcagctcgccccgcacagccccgtctatcaggtcggcgagttcgtcgagatcgacggcgagtgcgaccccgatctcctggtggcggcgttgcgtcaggtcatgggcgaggccgagagcgcccggctgcggttccgcgtgatcgacggtacgccgtggcagtacgtcgccgaggacggcgacgacccgatccaggtcgtggacctcggcgcggccgcggacccgcgcgccgcggcgctgggccgcatggcggccgacctcgaccggcccggcgacctgcgcgacggcccgctcgtcgagcaccacgtctacctgctcggcgagggccgggtcatctggtaccaccgcgcgcaccacatcgtctgcgacggcggcagcctcggcattgtcgcctcccgggtggccggcgtctattccgcgctcgcggccggtggtgacgtccggccgggtgcgctgccgccgctgtcggtgttgctgtcggccgccgacgcctacgagcgctccggcgaccgcgaccgggaccgcgagcactggcgctccgcgctggcgggcctgcccgccgagctgctcgcgggcgcgggccggccgcggccgctgcccggaccgccggtgcgccacgagcacgacctctccgcggcggaggcgggccggctgcgcgcgggggcgcggcggctgcggaccagcgtggcgcaggccggcatcgcggccgcggccctctaccagcaccggctcaccggcgcccgggacgtgctggtggcggtgcccgtcgccggccgcaccacccgcccggagttcgacgtgcccggcatgacgtcgaacgtggtgccggtgcgcctcgcggtcacgcccgccacgaccgtcggcgagctgctgcgcgacgtcgcccgtggtgtccgcgacggcctgcggcaccagcggtacccgtacccgaacatcgtggacgacctcggcctggccgaccgtgccgcgctgcgcccggtgaccgtcaacgccctggcgctgggacggccgctgcgcttcggctcggcggtgggtgtgcgctccggcctgtcggcgggcccggtggacgacgtcaccatcggcctctacgaaaaggtcagcggcggcggcatgcagacgatcgccgagctgaaccccgggcgcacggaccgcccggacgcggcggaggtctcccgctggttccgtacgctgctgcgcgggctggccgagagcgacgccggcgacccggtggcccgcatcgacatcgtcgacgagccc

SEQ ID NO: 6 - protein, 435 aa, Actinoplanes sp.
PDLRPLTPAQLAVWHAQQLAPHSPVYQVGEFVEIDGECDPDLLVAALRQVMGEAESARLRFRVIDGTPWQYVAEDGDDPIQVVDLGAAADPRAAALGRMAADLDRPGDLRDGPLVEHHVYLLGEGRVIWYHRAHHIVCDGGSLGIVASRVAGVYSALAAGGDVRPGALPPLSVLLSAADAYERSGDRDRDREHWRSALAGLPAELLAGAGRPRPLPGPPVRHEHDLSAAEAGRLRAGARRLRTSVAQAGIAAAALYQHRLTGARDVLVAVPVAGRTTRPEFDVPGMTSNVVPVRLAVTPATTVGELLRDVARGVRDGLRHQRYPYPNIVDDLGLADRAALRPVTVNALALGRPLRFGSAVGVRSGLSAGPVDDVTIGLYEKVSGGGMQTIAELNPGRTDRPDAAEVSRWFRTLLRGLAESDAGDPVARIDIVDEP

SEQ ID NO: 7 - DNA, 1305 bp, Streptomyces roseosporus
tcgcagcgcctcggcgtcaccgccgcccaacagagcgtctggctcgccggccagctggcggacgaccaccgcctgtaccactgtgcggcgtacctgtcactcaccgggtccatcgacccgcggacactcggcacggcggtccggcggaccctcgacgagaccgaggcgctgcgtacccggttcgtaccgcaggacggggaactgctgcagatcctcgaacccggtgccggacagctcctgctggaagccgacttctccggcgacccggaccccgagcgggcggcacacgactggatgcacgcggcgctcgccgcaccggtccgcctcgaccgcgccgggaccgccacccacgccctgctcaccctcggcccgtcccgccacctgctgtacttcggctaccaccacatcgcgctcgacggctacggtgccctgctccacctgcgccgcctcgcccacgtctacaccgccctcagcaacggggacgaccccggcccctgcccgttcggccccctggccggtgtcctcacggaggaggcggcctaccgtgactccgacaaccatcggcgcgacggggaattctggacccggtccctcgccggtgcggacgaggcccccgggctgagcgagcgggaggccggcgctctcgccgtcccgctgcgccgcaccgtggagctgtccggcgaacggacggagaagctggccgcctcggccgcggccactggagctcgctggtcgtcactgctcgtcgccgccaccgccgcgttcgtacgccgccacgctgccgccgacgacaccgtcatcggcctgcccgtcaccgcccggctcaccgggccggcgctgcgtaccccgtgcatgctcgccaacgacgtgccgctgcgcctcgacgcccggctcgatgccccgttcgccgcgctccttgccgacaccacccgcgccgtcggcacgctggcgcgccaccagcggttccgcggggaagaactccaccggaacctggggggcgtcggccgcaccgcgggcctggcgcgggtcaccgtcaacgtcctggcgtatgtcgacaacatccggttcggcgactgccgggccgtggtccacgagttgtcctcgggaccggtccgcgacttccacatcaactcctacggcacccccggcacccccgacggcgtccagctggtcttcagcggtaaccccgccctgtacacggccaccgatctggccgaccaccaggagcggttcctgcgcttcctcgacgctgtgaccgccgacccggacctgccgaccggaagacaccgcctcctgtcgccgggc

SEQ ID NO: 8 - protein, 435 aa, Streptomyces roseosporus
SQRLGVTAAQQSVWLAGQLADDHRLYHCAAYLSLTGSIDPRTLGTAVRRTLDETEALRTRFVPQDGELLQILEPGAGQLLLEADFSGDPDPERAAHDWMHAALAAPVRLDRAGTATHALLTLGPSRHLLYFGYHHIALDGYGALLHLRRLAHVYTALSNGDDPGPCPFGPLAGVLTEEAAYRDSDNHRRDGEFWTRSLAGADEAPGLSEREAGALAVPLRRTVELSGERTEKLAASAAATGARWSSLLVAATAAFVRRHAAADDTVIGLPVTARLTGPALRTPCMLANDVPLRLDARLDAPFAALLADTTRAVGTLARHQRFRGEELHRNLGGVGRTAGLARVTVNVLAYVDNIRFGDCRAVVHELSSGPVRDFHINSYGTPGTPDGVQLVFSGNPALYTATDLADHQERFLRFLDAVTADPDLPTGRHRLLSPG

SEQ ID NO: 9 - DNA, 1359 bp, Streptomyces fradiae
gcacaccgtgtggccgccacgtcggcccagaccgggatctggacggcgcagcgtctgcgcggggacgacaggctctacgcctgcggcctcttcctcgaactcgaccacgtggtggaggaggtgctgagcgaggcgatccgccgcgccgtcgccgacaccgaggcgctgcgcaccgcgttccgggaggacgcggacggcgcgctggagcagcacgtcctcgcccggccgccgagcacgcagacccgcctcttccacgccgacccgagcggcggaaccccctcccgctccgcgtccctggactggatggaccggcaacgggcgcaaccctgggacctcgcgtcgggcgacacctgccgtcataccctgatccccctcggcggcgaccgctcgctgctgcacctgcgttaccaccacctcgccctggacgggtacggcgccgcgctctatctggaccggctcgcggcggtctaccgcgcgctgcgcaccggccatcaaccgcccccctgcgcgttcgcgccgctggcccgcctggtcgaggaggaccacgcctaccggaactccgcccgtcaccgcgcggacgccaatcactggcgcgaccgcttcgcggacctcccgcgccccaccagcctcgccgacgccaccacgcccgcggcgcccaccacgcccgccacgcccgccgcgcccgccgcgcccgacgaactgcggcgcaccgtgcgcctgtccgccgcccggtccgccgcgctgcgccgtgcctcggaccggagcggccgaccctggcccgtgtacgccacggccgcggtggccgccttcctgagccgactcgcgccgggggaggaggtcgtcgtcggcctcccggtcaccgccagggtgacccccgccgcggtgcgcacaccggggatgctcgccaacgtcgtaccgcttcgcctgcccgtccggcagggcatgtcgacggcggagctgctggagctgaccgcggccgagatcagcaccacactgcgccaccagcgccaccgcaccgaggacatcgggcgggcgctcggactccacggcgctccgccagccaccacactcgtgaacgtcatggcgttcgccccggtcctcgacttcggcgactgccgggccccggtgcaccagctctcggccggaccggtggaggacctggtcgtcaacctcctcggcaccccgggcgacggcggcgagagcgacggcaccgagctggagatcactgtcgccgccaacccccgcctccactcggcggacgcggtggcctcgctggccgcgcggctcgcggagttcctcacgcacatggggcaggacgccgaggcgcccctcggccggacccggctgctcgacgcggag

SEQ ID NO: 10 - protein, 453 aa, Streptomyces fradiae
AHRVAATSAQTGIWTAQRLRGDDRLYACGLFLELDHVVEEVLSEAIRRAVADTEALRTAFREDADGALEQHVLARPPSTQTRLFHADPSGGTPSRSASLDWMDRQRAQPWDLASGDTCRHTLIPLGGDRSLLHLRYHHLALDGYGAALYLDRLAAVYRALRTGHQPPPCAFAPLARLVEEDHAYRNSARHRADANHWRDRFADLPRPTSLADATTPAAPTTPATPAAPAAPDELRRTVRLSAARSAALRRASDRSGRPWPVYATAAVAAFLSRLAPGEEVVVGLPVTARVTPAAVRTPGMLANVVPLRLPVRQGMSTAELLELTAAEISTTLRHQRHRTEDIGRALGLHGAPPATTLVNVMAFAPVLDFGDCRAPVHQLSAGPVEDLVVNLLGTPGDGGESDGTELEITVAANPRLHSADAVASLAARLAEFLTHMGQDAEAPLGRTRLLDAE

SEQ ID NO: 11 - DNA, 1296 bp, Streptomyces ghanaensis
tcggtgcgtcatggggtgctggccgcgcagcgagaggtctgggtggcccagcaactgcggccgctcagccctcggttcaactgcggcgttttcctggacgtcggcgaggccctcgacgccgccgtgctccgccgcgccgtgacccgtgccctggaggagacggagacgctgcgctcactgttcgccgaacaggacggcgacggcgagatcatccggaccacgcggccggcccccgacgactgcgtgacgacaatcgacgtgcgcgacgcggacgacccggtcgccgcggcacggcggtggatggacgccgacctggccgagccggtcgacctgcggcacgacccgagctaccggcacgtgctgttccggatcggcgagcggcgctccatcttctacttccgctaccaccacatcacgctcgacggtttcgggcagaccctgtacctgaaccgggtggccgacctctacacggccctggccaccgccaccgagccggacgcggccccgttcggcggcctggaccgcctgctggacgaggaacggcagtacgaggactccgccaggtgcgccgaggaccgggcccactggcacaccaccgcccggtccctcgccgagggacgcggcagcggcccggcggccgcctcggaccaggtgctccgcgacacggtgcggctgccgcgggaactgaccgacgcggtgtgcgcccacgcgcggtcgcacggttcgcgctggaccgcggtgatgctgggtgccgtggcggcctgtgcccggcggcggcagggcgacgacgcggtcgtgatcgacctgccggtgaccgcccgcacgacccgggccgcgctgacgacgcccggcatgatgtcgaacgtgctgccgctgcggctggaggtcgcgcgcgacgcggacctgcgcgccctcacggaggaggtgtcccgggcactgccggcgacactccggcaccagcgcttccgcggcgaggagctgtaccgcgagctcggcgcgggcggcgcgcggggacacctctcggtgaacgtgatgccgttcgaccaccaggtgcggttcggcaccgcgccggcgaccctgcaccaactggccaacggccaggtgcacgaggtggcgatcgacgtgtacgggacccccgacaagggcggcgacatccacgtcaccgtgcacgccaacgcccggacgcacaccgtcgaggacgtccggcagtggcaccgggagctgcgccgcatgctcgtccacctcctcggcggaccgggccgcacggtcggcgaggccgaactgctcgacgaggcc

SEQ ID NO: 12 - protein, 432 aa, Streptomyces ghanaensis
SVRHGVLAAQREVWVAQQLRPLSPRFNCGVFLDVGEALDAAVLRRAVTRALEETETLRSLFAEQDGDGEIIRTTRPAPDDCVTTIDVRDADDPVAAARRWMDADLAEPVDLRHDPSYRHVLFRIGERRSIFYFRYHHITLDGFGQTLYLNRVADLYTALATATEPDAAPFGGLDRLLDEERQYEDSARCAEDRAHWHTTARSLAEGRGSGPAAASDQVLRDTVRLPRELTDAVCAHARSHGSRWTAVMLGAVAACARRRQGDDAVVIDLPVTARTTRAALTTPGMMSNVLPLRLEVARDADLRALTEEVSRALPATLRHQRFRGEELYRELGAGGARGHLSVNVMPFDHQVRFGTAPATLHQLANGQVHEVAIDVYGTPDKGGDIHVTVHANARTHTVEDVRQWHRELRRMLVHLLGGPGRTVGEAELLDEA

SEQ ID NO: 13 - DNA, 1314 bp, Streptomyces refuineus
gcagaccgcgtggccgccacctcggcccagtccgggatctggacggcacagcggctgcgctcggatgaccggctctacacctgcggcctctacctcgaactcgaccacgtggtggaggaggtgctgggcgaggcgatcggccgtgcggtcgccgacaccgaggcgctgcgcaccgccttcggggaggacggggacggcgcgctggaacagcgcgtgctcgcgcggccgccggacacgcagacacggctgttccggctggacctgggcggagacgaccggccccgcgccgaggccctggactggatggaccggcagcaggcggaaccgtgggacctcgccgccggcgacacctgccggcacaccctgatccgcctcggcggccaccgcaccgtcctgcacctgcgctaccaccacctcgccctggacgggttcggtgccgcgctctacctggacaggatcgcggcggtgtaccgggcgctgcgcaccggccgggagacgcccccctgcaccttcgcgccgctggcccgcctcgtggaggaggaccgcgcctaccggcggtccgcccgccaccgcagggacgccgaccactggcggacgcgcttcgcggacctcccccgccccaccagcctcgccggcgccgccgcgcccgccgcgcccgccgcgctgcgccacacggtccgcgtgtccgcggccgacaccgccgcactgggcctgcgggcggaccggagcggcagcacctggccggtgttcgccacggccgcggtggccgccttcctgagccgcctcgcgccgggggaggaggtcgtcgtcggcttcccggtcaccgccagggtcacgcccgccgcggtgcgcacgccggggatgctggcgaacgtcgtgccgctccggatccgggtgcggcaggggatgtcgttcgccgcgctgctggaccggaccgcggccgagatcggcgccacgctgcggcaccagcgccaccgcaccgaggacatcggccgggcgctcggcctccccccgcacggcgcccagccggccccgaccctggtcaacgtcatggccttcgccccggtgctcgacttcggcgactgcctctcgccggtgcaccagctgtcggccggcccggtcgaggacctggcggtcaacctgctcggcacccccggggacggccgggagctggagatcaccgtcgccgccaaccccctgctccactcggaggacgcggtggcgtcgctggccgcgcggctggcggagttcctggcgcgcgcgggcgagcacgccgacgccccgatcggccggacacgcctgctcggcgcggcg

SEQ ID NO: 14 - protein, 438 aa, Streptomyces refuineus
ADRVAATSAQSGIWTAQRLRSDDRLYTCGLYLELDHVVEEVLGEAIGRAVADTEALRTAFGEDGDGALEQRVLARPPDTQTRLFRLDLGGDDRPRAEALDWMDRQQAEPWDLAAGDTCRHTLIRLGGHRTVLHLRYHHLALDGFGAALYLDRIAAVYRALRTGRETPPCTFAPLARLVEEDRAYRRSARHRRDADHWRTRFADLPRPTSLAGAAAPAAPAALRHTVRVSAADTAALGLRADRSGSTWPVFATAAVAAFLSRLAPGEEVVVGFPVTARVTPAAVRTPGMLANVVPLRIRVRQGMSFAALLDRTAAEIGATLRHQRHRTEDIGRALGLPPHGAQPAPTLVNVMAFAPVLDFGDCLSPVHQLSAGPVEDLAVNLLGTPGDGRELEITVAANPLLHSEDAVASLAARLAEFLARAGEHADAPIGRTRLLGAA

SEQ ID NO: 15 - DNA, 1296 bp, Streptomyces aizunensis
ggtgggctccgggaaatgatggcgggccagctcgcgatctggtacagccatcagttggcgcccgagaacccgtgcttcaacggtgccgagtacctggcgcttgacggagacgtggatctgggcctcctggtgaaggcctcgcagcggctgatggaagaggcggacgccgcccggctgcggatccgtgaagtggacgggcagccgaggcagtacttccacgacgtggaggactaccccgtcgaggtcatcgacatcagctccgaggccgatccccaggcggcggccgagagcctgatgtgggaagacctgcggggcgagcggggagcggccgaccgctccctctacaccatcaagatctacacggccggtccccggctcaccttctggtaccagcgggcctaccacgtgatcctggacggccgcagcgcgggcctggtggtcggccgcctgtcgcaggtgtacaacaccctgctccagggcggttccgtggaagagggcgccctgccctccagcaccgtcctgatggacgcggaacgcgagtaccggacctccgaggcccacgaggccgaccgggagtactggcgcggcgtcctcgcgggcctccccgaggccgagggcctcggcagcaactacggcggccgcgcccagcgcgcccccatccggttcgtggagagcgtcggcgacgccgtcgccacggacctgaagacggccgcccgcgggctggggacgaacttcgccggcctgatgatcagcgccgccgccctctaccagcaccacctcaccggacagcaggacgtggtcgtcggcgtcccggtcagcggccgctccggaacgcgcgacctcgccattccgttcatgaccaacaacgtccttccgatccgggtgacgatcgccccggacacctcggtcgccgacctcgtgcggcagaccacgcgcgccgtgatgaagggcctgcgccaccagcgctaccgctacgagcacatgctcaacgacgcgatgctcggcgagggcggtctctgggacctgctcatcaacgtgatgtccttcgacatctacgccctccccttcggcgactgcaccgtcaccgcgcacaacctctccagcggccccgtcgacagcacgcgcatcgacgtgtacgaccgctccggcctgaagatcgccgtcgacgtcaaccccgacgcccccgacctgtcgccgggcgacgaggtctgccgtcgcttcctggcgctcgcgcactggctcgtctcggtcgatcccgccgaaccggtcggccgctccggcctgctggacgcggac

SEQ ID NO: 16 - protein, 432 aa, Streptomyces aizunensis
GGLREMMAGQLAIWYSHQLAPENPCFNGAEYLALDGDVDLGLLVKASQRLMEEADAARLRIREVDGQPRQYFHDVEDYPVEVIDISSEADPQAAAESLMWEDLRGERGAADRSLYTIKIYTAGPRLTFWYQRAYHVILDGRSAGLVVGRLSQVYNTLLQGGSVEEGALPSSTVLMDAEREYRTSEAHEADREYWRGVLAGLPEAEGLGSNYGGRAQRAPIRFVESVGDAVATDLKTAARGLGTNFAGLMISAAALYQHHLTGQQDVVVGVPVSGRSGTRDLAIPFMTNNVLPIRVTIAPDTSVADLVRQTTRAVMKGLRHQRYRYEHMLNDAMLGEGGLWDLLINVMSFDIYALPFGDCTVTAHNLSSGPVDSTRIDVYDRSGLKIAVDVNPDAPDLSPGDEVCRRFLALAHWLVSVDPAEPVGRSGLLDAD

SEQ ID NO: 17 - DNA, 1293 bp, Actinomycete
gtggatcgtcgtcccgtctccgccgcccagctgggcatctgggtcgcgcagcaggtgctgccggacagtcctctgtacaactgcggctgctactacgagatcggcgcggccgatcccgggctgctcgaccgcgcggtccggcacacgctggccgagaccgaggcgctgcggtcgcgcttcgagacgatcgacgaccagctgtggcagctcgtcgggccggccgaccccgagccgctggaggtcgtcgacctgcgcgcggagcccgacccggaggcggccgcccggcgctggatgggggccgcgatggccgaggtccggcccctgggccgggccccgctgagccgccaggccgtgctgctgctcggcgcggaccgccggctgtggttccacggctaccaccacgccgtgctggacggcttcggccagtccgtctacgccgcccgggtggcgcaggtctacgccgccctggccgccggccggaccccgccggagcgggtcttcgcgacgctggacgaggcgcacgccgacgccgcggtggaccccgcgtcccgccggttcgccgccgaccgggactactggctcggcgccttcgccgaccggccggagccggtcgggctggccggccgggccggcgccgccgggccgacccagctgcgccggatccgcccgctgccgccggggtgcgccgcccggttcgcggcggcggccgaggcggtgggcagcacctggccggccgccgtgatcgccgcggtggccgcctactaccaccggatgaccgggcgcgaggagatcgtcttcgcgctgccgctggccggccgccgcggccgggcctcgctgagcacgcccggggccctggtcaacgtgctgccgatccggctctcggtgagctcccgggccaccttcgccgagctggcccggcaggccggccgccggctggccgacgtgctgcgccatcagcgcttccgcggcgagcagctgttccaggagctgggcctgtccggcgagcgcgcgttctgggggcccacggtcaacgtgatgggcttcggcggcgacctggccctgggcccggtcaccgcggtgccgcacccgctcgcgaccggcccggtccaggacctcaagatcaacttctacggtacgccggccacgggcgtccgcctcgaactcgacgccgacccggcccgctacgacgcggtggccgtcgcggagcaccaggaccggctgatccggctgctgacggcgctcgggcacgacccggcgacccggatcggcgccgtcgacctgctcgaccccgcc

SEQ ID NO: 18 - protein, 431 aa, Actinomycete
VDRRPVSAAQLGIWVAQQVLPDSPLYNCGCYYEIGAADPGLLDRAVRHTLAETEALRSRFETIDDQLWQLVGPADPEPLEVVDLRAEPDPEAAARRWMGAAMAEVRPLGRAPLSRQAVLLLGADRRLWFHGYHHAVLDGFGQSVYAARVAQVYAALAAGRTPPERVFATLDEAHADAAVDPASRRFAADRDYWLGAFADRPEPVGLAGRAGAAGPTQLRRIRPLPPGCAARFAAAAEAVGSTWPAAVIAAVAAYYHRMTGREEIVFALPLAGRRGRASLSTPGALVNVLPIRLSVSSRATFAELARQAGRRLADVLRHQRFRGEQLFQELGLSGERAFWGPTVNVMGFGGDLALGPVTAVPHPLATGPVQDLKINFYGTPATGVRLELDADPARYDAVAVAEHQDRLIRLLTALGHDPATRIGAVDLLDPA

SEQ ID NO: 19 - DNA, 1305 bp, Streptomyces sp. (Ecopia strain)
ggcggccgtcgtgagctgatggccggacagcttggcttatggcatgcgcagcaactgaatccggataatccgatctataacatgggtgaatacatagagattcgcggaaaggtggacacgagcttattcgaggcggctgtgcgaagggtcgtcctggaagtcgacggttttggtctgcgctttgagggaggtgctgacgaagttccgcggcaatattttggcctgcggagcgattggctgtttcatgtgatcgacgtgagcggcgaggaggacccccgttccagcgcggagagttggatgcgggcggacatgcgacgcccggtggatctccaggtcggtgaactcttcacccaggccatcatcaaggtggacgaagatctcttcttctggtatcagcgaatacaccacatcatcgcggacggactcgcgggaccccggatagcctcccgagtggctgcggtctacacggcactgtcggccggcgaacccctcgcggacagtgcgcctccctcgagttccgtactgatggacgccgatgccgactaccgggcgtccccagaattcgaactggaccggcagtactggacggagcgtctttccgatcgccctcaaacggtcagcttgagcggccaggaaccttccaccacaccccatgaactgacacggcatacgctccacatcccacccgacgccgctgcggaactcagaagctccgcccgtcggctgggaacgagcctctcgggtctggccgtagccgcgagcgccgcctacctgcatcgcgcaacgggacaagaggacatcattctcggggtccccgtaatgggcagaaaaaccgcgctgcgggacatccccggaatgacggcgaatatcgttcctctgcgcctcgctgtgcagccgaaggccacggtgagggagctcgtgaagcaggtatctcgcggagtacgagacgccttgcggcatcagcgataccgctacgaggacatcctcagagacctgaagctcgtggggcgcgacggactctaccccctactggtcaatatcgtctcctttgactacgatttgagatttggtgacgcccccagcattgcgcacgggctcggcggcataaacttcaacgacctgtcgatttccgtgtacgacaggtcgtccgacggaagcatgtccgtggttgtggacgccaatcccgacctttacagccgtggagcggtgcaagagcatgccgcgaaattcctcgacgtgatgaactggatggcgcgttccgctgcggaggaacgcatccaccagatcacgttgatgagccgctcc

SEQ ID NO: 20 - protein, 435 aa, Streptomyces sp. (Ecopia strain)
GGRRELMAGQLGLWHAQQLNPDNPIYNMGEYIEIRGKVDTSLFEAAVRRVVLEVDGFGLRFEGGADEVPRQYFGLRSDWLFHVIDVSGEEDPRSSAESWMRADMRRPVDLQVGELFTQAIIKVDEDLFFWYQRIHHIIADGLAGPRIASRVAAVYTALSAGEPLADSAPPSSSVLMDADADYRASPEFELDRQYWTERLSDRPQTVSLSGQEPSTTPHELTRHTLHIPPDAAAELRSSARRLGTSLSGLAVAASAAYLHRATGQEDIILGVPVMGRKTALRDIPGMTANIVPLRLAVQPKATVRELVKQVSRGVRDALRHQRYRYEDILRDLKLVGRDGLYPLLVNIVSFDYDLRFGDAPSIAHGLGGINFNDLSISVYDRSSDGSMSVVVDANPDLYSRGAVQEHAAKFLDVMNWMARSAAEERIHQITLMSRS

SEQ ID NO: 21 - DNA, 1338 bp, Streptomyces coelicolor
tcggttcggcacggtctgacgagcgcgcagcacgaggtgtggctcgcccagcagctggatccgcgtggcgcgcactaccggacgggatcctgcctggagatcgacggacccctggaccacgcggtgctgagccgcgccctgcggctcaccgtggccggtacggagacgctctgctcgcgcttcctcaccgacgaggagggccggccgtaccgcgcgtactgcccgcccgcgccggagggttcggccgccgtcgaggacccggacggggtgccgtacacccccgtgctgctgcgccacatcgacctctccggtcacgaggaccccgagggcgaggcccagcggtggatggaccgggaccgcgcgacgccgctgccgctggaccggcccggcctgagcagccacgcgctgttcacgctcggcgggggccggcacctgtactacctgggcgtccaccacatcgtgatcgacggcaccagcatggccctgttctacgagcggctggccgaggtgtaccgcgcgctgcgggacgggcgtgcggtgcccgcggccgccttcggggacacggaccggatggtcgcgggcgaggaggcctaccgcgcgtcggcgcggtacgagcgtgaccgggcctactggaccggcctgttcaccgaccgccccgagcccgtctcgctcaccgggcgcggcggcggccgggccctcgcgccgaccgtgaggagcctgggcctgcccccggagcgcacggaggtgctcggccgggccgccgaggcgaccggtgcgcactgggcgcgcgtggtcatcgccggtgtggccgccttcctgcaccggacgacgggcgcccgggacgtcgtggtgtcggtgccggtcaccgggcgctacggcgcgaacgcccggatcacccccggcatggtctccaaccggctgccgctgcggctggcggtgcgccccggcgagagtttcgcgcgggtggtcgagaccgtgtccgaggcgatgagcggcctcctggcgcacagccgcttccgcggcgaggacctcgaccgggagctgggcggcgcgggggtgtcggggcccaccgtcaacgtcatgccgtacatcaggccggtggacttcggcggtccggtcggcctgatgcgcagcatcagttcgggtccgaccaccgatctgaacatcgtgctgaccggcacccccgagtccggcctgcgcgtcgacttcgagggcaacccgcaggtgtacggcggccaggacctgacggtgctgcaggaacgcttcgtccggttcctggcggagctggcggccgaccccgcagccaccgtcgacgaggtcgcgctgctgacgccggac

SEQ ID NO: 22 - protein, 446 aa, Streptomyces coelicolor
SVRHGLTSAQHEVWLAQQLDPRGAHYRTGSCLEIDGPLDHAVLSRALRLTVAGTETLCSRFLTDEEGRPYRAYCPPAPEGSAAVEDPDGVPYTPVLLRHIDLSGHEDPEGEAQRWMDRDRATPLPLDRPGLSSHALFTLGGGRHLYYLGVHHIVIDGTSMALFYERLAEVYRALRDGRAVPAAAFGDTDRMVAGEEAYRASARYERDRAYWTGLFTDRPEPVSLTGRGGGRALAPTVRSLGLPPERTEVLGRAAEATGAHWARVVIAGVAAFLHRTTGARDVVVSVPVTGRYGANARITPGMVSNRLPLRLAVRPGESFARVVETVSEAMSGLLAHSRFRGEDLDRELGGAGVSGPTVNVMPYIRPVDFGGPVGLMRSISSGPTTDLNIVLTGTPESGLRVDFEGNPQVYGGQDLTVLQERFVRFLAELAADPAATVDEVALLTPD

SEQ ID NO: 23 - DNA, 1763 bp, Actinoplanes sp.
tggtcatcgacgccgccacccaacccaccgttcccgacgccttccgggcgcaggcgatcgcgcgccccggcgagcccgccctcgtggtgctccccggcgacccggacgccgagcccgtcaccctcacgtacgccgagctcgaccgccgcgccgcggcgcgggcggcctggctcgccgcccggttcccggccggggagcgcatcctcatcgccctgcccaccggcgccgagttcgtcgagctctacctggcgtgcctctacgccggcctggtcgccgtgccggcgcccccgcccggagggtcgtccggcgcctccgagcgcaccgtcggcatcgcggccgactgctcccccgccctggccgtcgtcaacgccgacgacgcggcgccgctcaccgccgtcctgcgcgagcgcggcctgtccggcctgccggtcggtgcgcttccgcccctcgcggcggaagcgatccgcccgccccgcgggccccggccggactcgctggccgtcctgcagtacagctcgggctccaccggctcgcccaagggcgtgatgctcagccaccgggccgtgctggccaacctccgcgcgttcgaccgcagcagcgggcacaacagcgacgacgtgttcggcagctggctgccgctgcaccacgacatgggcctgttcgccatgctcaccgcgggcctgctgaacggcgccggcgtcgtgctgatgtcgccgacggccttcgtccgccggccggcggactggctgcggatgatggaccgctaccgggtcaccatctccgccgcgcccaacttcgcgtacgacctgtgcgtgcgcgccgtgcgggacgagcagatcgccggcctcgacctgtcccgcatccgcacgctctacaacggatcggagccggtcaacccggccaccgtccgggcgttcaccgagcgcttcgcccccttcggcctgcacacccacgcggtgaacccctgctacggcatggccgagttcaccgcgtacgtgtcgacgaaggtcttcgaggcgccggcggtctttcttcccgccgaccctcgcgcgctggaggacgccgcgtcgccggccctgcgcccggccgaccccgccgcggcccgggagataccgggtgtcggccgggtgcccgacttcgaggtgctcatcgtcgacccggacgggctacggccgctgcccgagggccgggtcggcgagatctggctgcgcgggcccggcgcgggcgccggctactggggcaggaccgagctcaaccccggcatcttcgacgccaggcccgcgggcgacggccaggacggcggctgggtgcgaacgggtgacctgggtgcgctgaccggaggcgagctgttcctcaccggacgcctcaaggagctgctcatcgtgcacggccgcaacctggccccgcacgacctcgagcgggaggcccgggccgcgcacgacgcggtggaccaccagatcggggcggcgttcggggtgccggcgcccgacgagcggatcgtgctggtgcaggaggtgcatccgcgcacgccgctcgacgagctgccgcgggtggcgagcgccgtcagccgccggctcaccgtctccttcggcgtgccggtacgcaacgtgctgctggtgcggcgcggcacggtgcgccggaccacgagcggcaagatccgccggaccgcggtccgcgagcggttcctggccggcggcatcacggcgctgcacgccgagctcgagccggcgctgcggccggtgcaggcgggcgcgggccgatga

SEQ ID NO: 24 - protein, 587 aa, Actinoplanes sp.
MVIDAATQPTVPDAFRAQAIARPGEPALVVLPGDPDAEPVTLTYAELDRRAAARAAWLAARFPAGERILIALPTGAEFVELYLACLYAGLVAVPAPPPGGSSGASERTVGIAADCSPALAVVNADDAAPLTAVLRERGLSGLPVGALPPLAAEAIRPPRGPRPDSLAVLQYSSGSTGSPKGVMLSHRAVLANLRAFDRSSGHNSDDVFGSWLPLHHDMGLFAMLTAGLLNGAGVVLMSPTAFVRRPADWLRMMDRYRVTISAAPNFAYDLCVRAVRDEQIAGLDLSRIRTLYNGSEPVNPATVRAFTERFAPFGLHTHAVNPCYGMAEFTAYVSTKVFEAPAVFLPADPRALEDAASPALRPADPAAAREIPGVGRVPDFEVLIVDPDGLRPLPEGRVGEIWLRGPGAGAGYWGRTELNPGIFDARPAGDGQDGGWVRTGDLGALTGGELFLTGRLKELLIVHGRNLAPHDLEREARAAHDAVDHQIGAAFGVPAPDERIVLVQEVHPRTPLDELPRVASAVSRRLTVSFGVPVRNVLLVRRGTVRRTTSGKIRRTAVRERFLAGGITALHAELEPALRPVQAGAGR

SEQ ID NO: 25 - DNA, 1803 bp, Streptomyces roseosporus
gtgcctgccgtgagtgagagccgctgtgccgggcagggcctggtgggggcactgcggacctgggcacggacacgtgcccgggagactgccgtggttctcgtacgggacaccggaaccaccgacgacacggcgtcggtggactacggacagctggacgagtgggccagaagcatcgcggtgaccctccgacagcaactcgcgccggggggacgggcacttctgctgctgccgtccggcccggagttcacggccgcgtacctcggctgcctgtacgcgggtctggccgccgtaccggcgccgctgcccggggggcgccacttcgaacgccgccgtgtcgcggccatcgccgccgacagcggagccggcgtggtgctgaccgtcgcgggtgagaccgcctccgtccacgactggctgaccgagaccacggccccggctactcgcgtcgtggccgtggacgaccgggcggcgctcggcgacccggcgcagtgggacgacccgggcgtcgcgcccgacgacgtggctctcatccagtacacctcgggctcgaccggcaaccccaagggcgtggtcgtgacccacgccaacctgctggcgaacgcgcggaatctcgccgaggcctgcgagctgaccgccgccactcccatgggcggctggctgcccatgtaccacgacatggggctcctgggcacgctgacaccggccctgtacctcggcaccacgtgcgtgctgatgagctccacggcattcatcaaacggccgcacctgtggctacggaccatcgaccggttcggcctggtctggtcgtcggctcccgacttcgcgtacgacatgtgtctgaagcgcgtcaccgacgagcagatcgccgggctggacctgtcccgctggcggtgggccggcaacggcgcggagcccatccgggcagccaccgtacgggccttcggcgaacggttcgcccggtacggcctgcgccccgaggcgctcaccgccggctacgggctggccgaggccaccctgttcgtgtcgaggtcgcaggggctgcacacggcacgagtcgccaccgccgccctcgaacgccacgaattccgcctcgccgtacccggcgaggcagcccgggagatcgtcagctgcggtcccgtcggccacttccgcgcccgcatcgtcgaacccggcgggcaccgtgttctgccgcccggccaggtcggcgagctggtcctccagggagccgccgtctgcgccggctactggcaggccaaggaggagaccgagcagaccttcggcctcaccctcgacggcgaggacggtcactggctgcgcaccggcgatctcgccgccctgcacgaagggaatctccacatcaccggccgctgcaaagaggccctggtgatacgaggacgcaatctgtacccgcaggacatcgagcacgaactccgcctgcaacacccggaacttgagagcgtcggcgccgcgttcaccgtcccggcggcacctggcacgccgggcttgatggtggtccacgaagtccgcaccccggtccccgccgacgaccacccggccctggtcagcgccctgcgggggacgatcaaccgcgaattcggactcgacgcccagggcatcgccctggtgagccgcggcaccgtactgcgtaccaccagcggcaaggtccgccggggcgccatgcgtgacctctgcctccgcggggagctgaacatcgtccacgcggacaagggctggcacgccatcgccggcacggccggagaggacatcgcccccactgaccacgctccacatccgcaccccgcgtaa

SEQ ID NO: 26 - protein, 600 aa, Streptomyces roseosporus
VPAVSESRCAGQGLVGALRTWARTRARETAVVLVRDTGTTDDTASVDYGQLDEWARSIAVTLRQQLAPGGRALLLLPSGPEFTAAYLGCLYAGLAAVPAPLPGGRHFERRRVAAIAADSGAGVVLTVAGETASVHDWLTETTAPATRVVAVDDRAALGDPAQWDDPGVAPDDVALIQYTSGSTGNPKGVVVTHANLLANARNLAEACELTAATPMGGWLPMYHDMGLLGTLTPALYLGTTCVLMSSTAFIKRPHLWLRTIDRFGLVWSSAPDFAYDMCLKRVTDEQIAGLDLSRWRWAGNGAEPIRAATVRAFGERFARYGLRPEALTAGYGLAEATLFVSRSQGLHTARVATAALERHEFRLAVPGEAAREIVSCGPVGHFRARIVEPGGHRVLPPGQVGELVLQGAAVCAGYWQAKEETEQTFGLTLDGEDGHWLRTGDLAALHEGNLHITGRCKEALVIRGRNLYPQDIEHELRLQHPELESVGAAFTVPAAPGTPGLMVVHEVRTPVPADDHPALVSALRGTINREFGLDAQGIALVSRGTVLRTTSGKVRRGAMRDLCLRGELNIVHADKGWHAIAGTAGEDIAPTDHAPHPHPA

SEQ ID NO: 27 - DNA, 1785 bp, Streptomyces ghanaensis
atggtcaacgtcagtgaagcgcggagtgtccccgaactcctgcggcatcacgcgagttcggcacccgaccgggaggcgctgcgctacctgcgcgacaccacggggacggacgggaccccgctcacctaccgggaagtggaccgcgctgccgccgccgtggcacggcgcctctcccggagcttcgaggcgggcgaccggctgctgctcctgcactccttcggcccggacttcatcgtgggcttcctcgcctgcctctacgccggcatggtggccgttcccgcgccgctgcccggcagataccgccatgaacgcagacgggtgctgagcatcgcccacgacagcggcgccgtcgcggtgctcaccgacgacgcgagctccgcggaggtcggcgagtggatgcgcgaggagggcctggacggcctccccctcatcgccaccgactggtccgccgaggagcccggtgccttcaccccggccgcggacctcggacgcgagacgctcgccatgctccagtacacctccggctccacgggcgagccgaagggcgtgatggtgacgcacgggaacctgctgcggaacgtcaccgcgctgagccgtgcgttcggcctcgacgagcacacccacttcggcggctggatcccccatttccacgacatgggcctgatcgggctgctgctgccctccctcttcctgcgcagcaggtgcgtgctgatgagcccgtccgccttcatccgccgaccgcacacctggctgaagatgatcgacgacttcgacgtcgcgtggtcggcggcccccgatttcgcctacgaactgtgctgccgacgagtcaccgacgagcagctcggcagcctcgacctctcgcgctggcgctacgcgggcaacggctcggaacccatccacgccggcaccatcaccgccttcgccgagcggttcgccgccgccgggttccgcgccgagtcgctgtccccgtgctacggcctcgccgagtcgacggtctacgtctccggcggtccctccgcccggatcaccgcggtcgacgcccagtcgctggaggaccaccggctcggcgaggccgtaccgggacggccgcaccgctcgctggtgagctgcggcgcgccggcggacgtcgacctccggatcgtcgacccgcggaccggggacccgctgccggacggcgcggtcggggagatctggctgcggggaggcagcgtcgccgtcggctactgggacaaccccgcggcgtccgccgagaccttcggcgccgtcatcgacggggtggagggccgctatctgcggaccggtgacctcggcgcgctgtacgacggggagctgtacgtcacgggccggatcaaggaaatgatcaccgtgcacgggaggaacgtctacccacaggacgtcgagcaggaactgcgcgccgcccaccaggaactcgccggctgcgtcggcgccgtcttcgccctctcggatacgggccccgacccggtcctggtcgtgtcccacgaggtccgggcgggcctcggtgcggacgtactggaggcgctggcccgggacatgaagcagacggtggcccgcgagatgggcatgacggcgtcgtgcgtggtgctcctgcgccgcgggacggtacgccggacgacgagcggcaagatccagcgcgacgcgatgcggaagctgttccgggacggcgagctgaagccccttcacacgcactggcacatgccgaggcagcgcgccgcggtccacgggagcagctcggcgcagagcctggccgaggagtccacggtatga

SEQ ID NO: 28 - protein, 594 aa, Streptomyces ghanaensis
MVNVSEARSVPELLRHHASSAPDREALRYLRDTTGTDGTPLTYREVDRAAAAVARRLSRSFEAGDRLLLLHSFGPDFIVGFLACLYAGMVAVPAPLPGRYRHERRRVLSIAHDSGAVAVLTDDASSAEVGEWMREEGLDGLPLIATDWSAEEPGAFTPAADLGRETLAMLQYTSGSTGEPKGVMVTHGNLLRNVTALSRAFGLDEHTHFGGWIPHFHDMGLIGLLLPSLFLRSRCVLMSPSAFIRRPHTWLKMIDDFDVAWSAAPDFAYELCCRRVTDEQLGSLDLSRWRYAGNGSEPIHAGTITAFAERFAAAGFRAESLSPCYGLAESTVYVSGGPSARITAVDAQSLEDHRLGEAVPGRPHRSLVSCGAPADVDLRIVDPRTGDPLPDGAVGEIWLRGGSVAVGYWDNPAASAETFGAVIDGVEGRYLRTGDLGALYDGELYVTGRIKEMITVHGRNVYPQDVEQELRAAHQELAGCVGAVFALSDTGPDPVLVVSHEVRAGLGADVLEALARDMKQTVAREMGMTASCVVLLRRGTVRRTTSGKIQRDAMRKLFRDGELKPLHTHWHMPRQRAAVHGSSSAQSLAEESTV

SEQ ID NO: 29 - DNA, 1806 bp, Streptomyces refuineus
gtggctcacgtgagcggacccccagcagacccgccggccggctcccacctggtggccgcgatccgcgcgacggccgaggccgaccccgagcgcaaggccgtcggcttcgtccgggatccggaacgcgaaggtgaggaggcgctgcggagctactcctggctcgacgacagggcccgccgcatcgccgtcctcctccgcggggcgcggctcggcgcgggctcgcgcgtcctgctgctcttcccgcagtccgcggagttcgcggcggcctacgccggatgcctctacggggggatggtcgccgtccccgcgcccctgcccacgggaacctccctggagaccgcacgcgtcgccggcatcgcccgggacgccggggcgggcgccgtcctcaccgtctccgacaccgaggcggaggtccggcggtgggcggccgagaccggtctgggcgacctgcccctgttctccgtcgacgaactgcccgacgacaccgacccgggggagtggcgggagccggagatccgggccggcaccgtggcggtgctgcagtacacctccggctccaccggcagccccaagggggtcgtcgtcacccacggcgcgctcgccgacaacgtccgcagcctcctgtccgggttcgacctgggaaccggcgcccggctgggcggctggctgccgatgtaccacgacatggggctgttcgggctgctgagcccggcgctgttcagcggcggcgccgccgtgctgatgagcggcagcgccttcctgcgcaggccgcacctgtggccgacgctgatcgaccgcttcggcgtggtcttctccgcggcgcccgacttcgcctacgactactgcgtacggcgggtggagcccgagcaggtggaccggctcgacctctcgcgctggcgctgggcggccaacggctcggagcccatccgggccgagacgctccgcgccttcaccaaggagttcgcccccgcggggctgccccacgacgcgatgaccccctgctacggactggccgaggcgaccctgctggtctccctgtcggcgggcgagctgcgcacccggcgggtggacgccgcggcactggagaaccaccgcttcgtcgaggcggccgcgggccgcccgtcccgcgaggtcgtctcgtgcggccggcccccggccctggaggtccgcgtggccgaccccgcgaccggagagcccgtcacgggcgatgcggtgggcgagatccaggtgcggggcgcgagcgtggccggcggctactggcggaaaccggaggcgaccgccgagacgttcgtcacggccgcggacggctccgggccctggctgcgcaccggcgacctcggcgccctgtacgagggcgagctgtacgtcaccggccgcatcaaggaactcctcatcgtgcacggccgcaacatctacccgcacgacgtcgagcgcgaactgcgcgcccaccacgacgagctcggcgcgatcggcgccgtcttctccgtccccacggaggagggcgaggccgtcgtggtcacgcacgaggtggtcccgtccgtccgggacgaccggggccccgcgctggtgacggcggtacgggcgacgctcgcccgggagttcggcctggcaccggccggggtggtgctggtgcgccgcggccgcaccccgcgcaccagcagcggcaaggtgcagcgccgcctggccgcccggctcttccgcaccggggaactcgcccaggtccacgccgaccccggtgcccaccggctcgtggcggcgctccgcgaggcggacggcctgcgcgacgcccccgcgtccacgacatga

SEQ ID NO: 30 - protein, 601 aa, Streptomyces refuineus
VAHVSGPPADPPAGSHLVAAIRATAEADPERKAVGFVRDPEREGEEALRSYSWLDDRARRIAVLLRGARLGAGSRVLLLFPQSAEFAAAYAGCLYGGMVAVPAPLPTGTSLETARVAGIARDAGAGAVLTVSDTEAEVRRWAAETGLGDLPLFSVDELPDDTDPGEWREPEIRAGTVAVLQYTSGSTGSPKGVVVTHGALADNVRSLLSGFDLGTGARLGGWLPMYHDMGLFGLLSPALFSGGAAVLMSGSAFLRRPHLWPTLIDRFGVVFSAAPDFAYDYCVRRVEPEQVDRLDLSRWRWAANGSEPIRAETLRAFTKEFAPAGLPHDAMTPCYGLAEATLLVSLSAGELRTRRVDAAALENHRFVEAAAGRPSREVVSCGRPPALEVRVADPATGEPVTGDAVGEIQVRGASVAGGYWRKPEATAETFVTAADGSGPWLRTGDLGALYEGELYVTGRIKELLIVHGRNIYPHDVERELRAHHDELGAIGAVFSVPTEEGEAVVVTHEVVPSVRDDRGPALVTAVRATLAREFGLAPAGVVLVRRGRTPRTSSGKVQRRLAARLFRTGELAQVHADPGAHRLVAALREADGLRDAPASTT

SEQ ID NO: 31 - DNA, 1743 bp, Streptomyces aizunensis
atgaccctcgaacccagcgtgctccatctgctgcgccggcacgccgtcgaccgggcagagcggaccgccgtcaccttcgtccacgacttcgacgcggccgacggctcgcggagcctgaactacgccgaactcgacgcggaggcacgtcgcgtcgcgtcctggctccaggagcgctgtgcgcccggagaccgggtgctgctgctgcacccggccggtctgcccttcgtcaccgcgttcctcgcctgcctctacgcgggtgtcatcgcggtgccgtctccgatgccgggccagttccagtaccagcagcgccgcgtgacgacgatcgcccgcgatgccggtgtcagcgtggcgctcaccgacacgggccagctgcccgaggcgcagcagtggatggccgacacccgcctcgaactgccggtcgccgcgagcgacgcccccggcttcggtgacgcgtcgcgctggcgcgaccccggcgccaccgcccaggacgtggtgctgctgcagtacacctccggctcgaccggtgaccccaagggcgtcatggtcacgcacgccaacctgctgcacaacgccgacagcctgagccgttccctcggcttcaccgaggacaccaacttcggcggctggatcccgctctaccacgacatgggcctgatggggcagctgctgccgggtctcttcctgggcagcagcgtcgcgctgatgtcgccgatggcgttcctcaagcgcccgcaccactggctcgcgctgatcgaccgctacgacatcggcttctccgccgcgcccaacttcgcgtacgagctgtgcctgcgccgggtcaccgacgcgcagatcgccgcactcgacctgtcgcgctggcagttcgccgccaacggctccgagccgatccaggccagcaccctgcgggagttcgcggagcgcttcggcccggccggcttccgggccgagcagctcgccccgtgctacggcatggccgaggcgacggtcttcatctccggccgctcgacccggccgccgcggatccgcgccgtcgacccgcaggcgctggagaagcacgtcgtccaggaccccgagccgggcggcctcgtgcgcgaactcgtcggctgcggcgacgtacccgacctcgacgtgcgcatcgtcgaagcgggcacgcgcacggtgctgacggacggcacgaccggcgagatctggctgcgcgggccgagcgtcgcggccggctactggaaccggccggaggtgaccgaggagatcttccgcgcccacaccgccgacggcgacgggccctacatgcgcaccggcgacctcggagtgctgctcgacggcgaaatctacgtcacgggccgcaccaaggacctgctgatcgtcaacggccgcaacctctacccgcacgacctcgaacacgaactgcggctctcccacgccccgttggcgaccctcgccggtacggcgttcaccgtccccgccccgcaggaagaggtcgtggtcgtgcacgaggtgcgcggccgcttcagccaggaagagctgcgcgagctggccatcggcatgcgcgccaccgtgcaccgcgagttcggcgtgcacaccgcgggcatcgtgctgatgcggcccggcacggtccgcaagaccaccagcggcaaggtgcagcgcgccgagatgcgcggcctgttcctcgcgggcgccctcgccccgctgtacgaggagatggcgcccggagtccaggcggcgatggccggagccgccgggtga

SEQ ID NO: 32 - protein, 580 aa, Streptomyces aizunensis
MTLEPSVLHLLRRHAVDRAERTAVTFVHDFDAADGSRSLNYAELDAEARRVASWLQERCAPGDRVLLLHPAGLPFVTAFLACLYAGVIAVPSPMPGQFQYQQRRVTTIARDAGVSVALTDTGQLPEAQQWMADTRLELPVAASDAPGFGDASRWRDPGATAQDVVLLQYTSGSTGDPKGVMVTHANLLHNADSLSRSLGFTEDTNFGGWIPLYHDMGLMGQLLPGLFLGSSVALMSPMAFLKRPHHWLALIDRYDIGFSAAPNFAYELCLRRVTDAQIAALDLSRWQFAANGSEPIQASTLREFAERFGPAGFRAEQLAPCYGMAEATVFISGRSTRPPRIRAVDPQALEKHVVQDPEPGGLVRELVGCGDVPDLDVRIVEAGTRTVLTDGTTGEIWLRGPSVAAGYWNRPEVTEEIFRAHTADGDGPYMRTGDLGVLLDGEIYVTGRTKDLLIVNGRNLYPHDLEHELRLSHAPLATLAGTAFTVPAPQEEVVVVHEVRGRFSQEELRELAIGMRATVHREFGVHTAGIVLMRPGTVRKTTSGKVQRAEMRGLFLAGALAPLYEEMAPGVQAAMAGAAG

SEQ ID NO: 33 - DNA, 1767 bp, Actinomycete
atggtggagctgggctcggccgaaagcattccggcggtgctgcgccggcacgcggagaatacgcccgaccgcgccgcccacgcctttgtcaccgacctcgacgaggccggcggcgtcgcctggctcagccacgccgagctggaccgccgggcccgggccgtggccgcgcagctgtccgcgcacgccgctcccggcgaccggatgctgctgctgcacccggccggcccggacttcctgatcgcgctgctcggctgcctgcacgccggtctgatcgcggtgccgtcgccgctgcccggccgctacgcccatcagcggcgccgggtccggctgatcgcggccgacgccgacgtgacggccgtgctgaccgaccgggccacccgcgcggaggtcgtcgagtgggccgccgagcagggactgcccgacatcgcggtgctgacccccgacccggaggccgacccgggtgactggcagccgccgccgctgagccgggacacggtcgccgtcctgcagtacacctccggctccaccggcaaccccaagggcgtggtcatcgaccacggcaacatcctgagcaacgccgccacgatcatcgcggtgaccgggatccggcccggcaccgtgatcggcgggtggctgccgcacttccacgacatgggcctgatgggcctgctgctgccgccgctgctggccggggcgacgacggtgctgagcagccccgtctcgttcctgaagcgcccgctgagctggctgcggatgatcgaccggtacggcgtcgagatcaccgccgcccccgacttcgcctacgacctgtgcgtcgccaaggtcaccgacgccgagctggccacgctcgacctgtcccgctggcgggtcgccatcaacggctccgagccggtccgggccgccgtgctcacccggttccggcagcgcttcgccgccgccgggctgcggcccgaggtgctgaccccgagcttcggcatggccgaggcgacgctgttcgtctccggcgacccggccaccccgttcgtcgtccgccgcgtcgacaccgaccggctggcccggcaccggttcgagccggccccggacggcgggccgggccgcgacgtggtggcctgcggcgcgccggccggcgtcgaggtgcgcatcgtcgaccccggcagcggcgacccgttgccggacggcgccgtcggcgagatctggctgcgcggcccgtcgatcggccgcggctactgggggcgcgccgcgaacaccgcgggcttcggcgcggtcaccagcatcggcgacgccgggtacctgcggaccggcgacctgggcaccctgtacgagggccagctctacgtcaccggccgccgcaaggacatgctggtgctgcgcggccgcaactactacccgcaggacatcgagcacgagctgcgggcgcaccaccccgagctggccgggcgcgtcggcgcctgcttcgccgtgcgatcgcgcgacggggcgggcggcggcgaggaggtcctcgtggtcacccacgaggtgcgcgggatctccgatccggaccggctgcgtacccttgccggggccatgcggctcacggtggcccgggagttcggcgtgccgagcgctgcggtgctgctgctgcgccccggcgcggtggcccgtaccaccagcggcaagatccagcgctcggcgatgcgggagctgttcgagaccggcgcgctggagccggtcggcggcgaggtggacgaccggctggtcgccaccgcggcgctgggcgcggcccgatga

SEQ ID NO: 34 - protein, 588 aa, Actinomycete
MVELGSAESIPAVLRRHAENTPDRAAHAFVTDLDEAGGVAWLSHAELDRRARAVAAQLSAHAAPGDRMLLLHPAGPDFLIALLGCLHAGLIAVPSPLPGRYAHQRRRVRLIAADADVTAVLTDRATRAEVVEWAAEQGLPDIAVLTPDPEADPGDWQPPPLSRDTVAVLQYTSGSTGNPKGVVIDHGNILSNAATIIAVTGIRPGTVIGGWLPHFHDMGLMGLLLPPLLAGATTVLSSPVSFLKRPLSWLRMIDRYGVEITAAPDFAYDLCVAKVTDAELATLDLSRWRVAINGSEPVRAAVLTRFRQRFAAAGLRPEVLTPSFGMAEATLFVSGDPATPFVVRRVDTDRLARHRFEPAPDGGPGRDVVACGAPAGVEVRIVDPGSGDPLPDGAVGEIWLRGPSIGRGYWGRAANTAGFGAVTSIGDAGYLRTGDLGTLYEGQLYVTGRRKDMLVLRGRNYYPQDIEHELRAHHPELAGRVGACFAVRSRDGAGGGEEVLVVTHEVRGISDPDRLRTLAGAMRLTVAREFGVPSAAVLLLRPGAVARTTSGKIQRSAMRELFETGALEPVGGEVDDRLVATAALGAAR

SEQ ID NO: 35 - DNA, 2169 bp, Streptomyces fradiae
ttgaccgtccgggcggagcaccggaaagcgtcgaccctgccgccggggaacccggccgtcagcagcggcgactccgcgtcccgccgggagaagagggccgccgctgggagcagttcttcggcggacccgctggccggcccccacctggtggccgcgatctccgcgacggccgaggccgacccggggcgcaaggccgtcggtctcgtccgggatccggagcgcgagggcgaggaggcgctgcggagctacgcctggctcgacgacaccgcccgccgcatcgccgtcctcctgcgtgcggccgggctggaaacgggcgcacgcgtgctgctgctcttcccgcagtccgcggagttcgcggcggcctacgccgggtgtctctacgcgggcatggttgccgtccccgcgccccttccgaccggcacctcccatgaggccgcacgcgtcgtcggcatcgcgaaggactccgaggcaggcgccgtcctcaccgtctccgaaaccgaggcggacgtccggcaatgggcggcccgcaccggcctgggcgcgctgcccctccactgcgtcgacgaactgcccggcgacgccgaccccgacacgtggcgggaaccggagatccgggccgacaccgtggcggtcctccagtacacctccggctccaccggcagccccaagggggtcgtcgtcacccacggcgcgctcgccgacaacgtgcgcagcctgctcacgggcttcgatctgggatccggcgcccggctgggcggctggctgccgatgtaccacgacatggggctgttcggcctgctgagcccggcactgttcagcggcggagccgccgtgctgatgagcggcagcgccttcctgcgccgcccgtcccagtggctgaggctgatcgaccgcttcggcctcgtcttctcggcggcgcccgacttcgcctacgactactgcgtacggcgggtgagacccgaggagacggacgggctcgacctgtcgcgctggcgctgggcggccaacggctccgagcccatccgcgccgagacgctgcgcgccttcgccaaggagttcgccccggccggactccacccgaacgccaccaccccttgctacggactggccgaggcgaccctgctggtgtccctgcccacgggtgagctgcgcacccgacgggtggacgtcgcggaactggagaaccaccgcttcgtcgaagcggccgtgggacgcccctcccgcgagatcgtgtcctgcggccggcccccgtccctggagatccgcgtcgtcgaccccgcgaccggcaagtccgtcacgggcggcgacggagccggcgagaccagggtgggcgagatcagagtgcgcggcgcgagcgtcgccaggggctactggcagaaaccggaggcgaccgccgagacgttcgtcatggacgcggacggctccgggccctggctgcgcaccggcgacctcggcgctctgtacgagggcgagctgtacgtcaccggccgtatcaaggaactcctcatcgtgcacggccgcaacatctacccccatgacatcgagcacgaactgcgcgcccgccacgccgaactcggcgctgtcggggccgccttctccctcagcaccgaatcgggcgaggttgtggtcgtcacccatgaggtgaaccccaccgtccggcccgagcagggtcccgagctggtgaccgccctgcgtgcgacgctcgcgcgggagttcggcctcgccccggccggggtggtgctggtgcgccgcggccgcatcccgcgcaccagcagcggcaaggtgcaacgccgcctgaccgcccggctgttcagcacgggggaactcgcccaggtccatgccgaccccggcgcccaccgcctcctggcggaactcagggaggcgcacgaccgcggcggcgccttcccgcccccctccccgcccgccagccaggaccccgaggccctgcggcagcggctgcgcgagctgtgcgccgactgtctcggcgtccccgtggactccctcgccacggacgcccccctcaccgactacgggatgacctccgtcaccggcaccgccctgtgcgggatggtggaggagtacctggacgtcgaatgcgacctggaactgctctggcaggagccgacgatcgacgggctcgcctcccggctggcctcgcgcaccgtgcgc

SEQ ID NO: 36 - protein, 723 aa, Streptomyces fradiae
LTVRAEHRKASTLPPGNPAVSSGDSASRREKRAAAGSSSSADPLAGPHLVAAISATAEADPGRKAVGLVRDPEREGEEALRSYAWLDDTARRIAVLLRAAGLETGARVLLLFPQSAEFAAAYAGCLYAGMVAVPAPLPTGTSHEAARVVGIAKDSEAGAVLTVSETEADVRQWAARTGLGALPLHCVDELPGDADPDTWREPEIRADTVAVLQYTSGSTGSPKGVVVTHGALADNVRSLLTGFDLGSGARLGGWLPMYHDMGLFGLLSPALFSGGAAVLMSGSAFLRRPSQWLRLIDRFGLVFSAAPDFAYDYCVRRVRPEETDGLDLSRWRWAANGSEPIRAETLRAFAKEFAPAGLHPNATTPCYGLAEATLLVSLPTGELRTRRVDVAELENHRFVEAAVGRPSREIVSCGRPPSLEIRVVDPATGKSVTGGDGAGETRVGEIRVRGASVARGYWQKPEATAETFVMDADGSGPWLRTGDLGALYEGELYVTGRIKELLIVHGRNIYPHDIEHELRARHAELGAVGAAFSLSTESGEVVVVTHEVNPTVRPEQGPELVTALRATLAREFGLAPAGVVLVRRGRIPRTSSGKVQRRLTARLFSTGELAQVHADPGAHRLLAELREAHDRGGAFPPPSPPASQDPEALRQRLRELCADCLGVPVDSLATDAPLTDYGMTSVTGTALCGMVEEYLDVECDLELLWQEPTIDGLASRLASRTVR

SEQ ID NO: 37 - DNA, 273 bp, Actinoplanes sp.
atgtccgagaccgacctgtccgccgcccggcacacgcccgagcagatccgctcctggctgatcgaccggatcgcctactacgtgatgctgccgacccaggagatcgagccggacgtgtccctggccgagtacggcctggactcggtgtacgcgttcgcgctctgcggcgagatcgaggacacgctcggcatcccgatcgagccgaccctgctgtgggacgtcgacaccgtcgccaccctcaccgcccacctcgccgaccgcgtcaaccgataa

SEQ ID NO: 38 - protein, 90 aa, Atinoplanes sp.
MSETDLSAARHTPEQIRSWLIDRIAYYVMLPTQEIEPDVSLAEYGLDSVYAFALCGEIEDTLGIPIEPTLLWDVDTVATLTAHLADRVNR

SEQ ID NO: 39 - DNA, 270 bp, Streptomyces roseosporus
atgaacccgcccgaagcggtcagcacgcccagcgaggtcaccgcgtggatcaccggacagatcgccgagttcgtgaacgagacacccgaccggatcgccggtgacgcacccctgaccgaccatggcctcgactccgtctccggagttgccctctgcgcgcaggtcgaggaccgctacgggatcgaggtcgacccggagctgctgtggagcgtccccacactcaacgagttcgtccaggcactgatgccccagttggccgaccgcacctga

SEQ ID NO: 40 - protein, 89 aa, Streptomyces roseosporus
MNPPEAVSTPSEVTAWITGQIAEFVNETPDRIAGDAPLTDHGLDSVSGVALCAQVEDRYGIEVDPELLWSVPTLNEFVQALMPQLADRT

SEQ ID NO: 41 - DNA, 273 bp, Streptomyces ghanaensis
atggaagcagcacagactccccgaacggccgccgaactcggcgactggctcacccgcacggtggccgactacgtcaggtgtgatccggcggagatcgacccggacgtgccgctgtccgattacggcctcgactcgatctcggcgaccacggtgtgtgccgacatcgaggaccacttcggtctgcccgtcgaagtgacgctgatctgggaccaccccacgataggcaaactgtcgcaggcactggccgaggagctcgaaaccgccgtccgctga

SEQ ID NO: 42 - protein, 90 aa, Streptomyces ghanaensis
MEAAQTPRTAAELGDWLTRTVADYVRCDPAEIDPDVPLSDYGLDSISATTVCADIEDHFGLPVEVTLIWDHPTIGKLSQALAEELETAVR

SEQ ID NO: 43 - DNA, 300 bp, Streptomyces refuineus
atgtccctgtccccgccttcttcgtccccgccttcttccccgcccccttctccgccgcacgaccccgacgccctgcggcagtggctgcgcgagcagtgcgccgactgcctcggcgtccccccggcatccctcgccaccgacgtccccctcaccgactacggcatgacctccgtcaccgggaccgccctgtgcggcatggtggaggaccacctggacgtcgagtgcgacctgagcctgctctggcaggagcagacgatcgacggcatcacctcccggctggcctcgcgcaccgcgcgctga

SEQ ID NO: 44 - protein, 99 aa, Streptomyces refuineus
MSLSPPSSSPPSSPPPSPPHDPDALRQWLREQCADCLGVPPASLATDVPLTDYGMTSVTGTALCGMVEDHLDVECDLSLLWQEQTIDGITSRLASRTAR

SEQ ID NO: 45 - DNA, 276 bp, Streptomyces aizunensis
atgtccgacatcaccgcccccccggccacggccgatgccgcagaggtccgcacctggctgcgcgaatgcgtggccacctacgtccggcttcccgccgaggacatcgacgtgaacctgccgctgtccgagtacggtctcgactccgtgtacgtgctcagcctgtgcgccgacatcgaggaccgctacggcatcgaggtcgagcccaccctgctctgggaccaccccgccatcggcccgatcgccgacgcgctgaccccgctgctcgccgctcgctag

SEQ ID NO: 46 - protein, 91 aa, Streptomyces aizunensis
MSDITAPPATADAAEVRTWLRECVATYVRLPAEDIDVNLPLSEYGLDSVYVLSLCADIEDRYGIEVEPTLLWDHPAIGPIADALTPLLAAR

SEQ ID NO: 47 - DNA, 267 bp, Actinomycete
atgtccgacctcaccgccgtccccacgccggagagcctgcgcgcctggctcgtcgactgcgtcgccgaccacctcggccgcgcgcccgccggcatcgccaccgacgtgccgctgaccacgtacggcctggactccgtctacgcgttgtcgatcgccgcggagctcgaggaccacctggacgtctcgctcgatccgaccctgatctgggaccacccgacgatcgacgccctcagcgcggccctggtggccgagctgcgttccgcctga

SEQ ID NO: 48 - protein, 88 aa, Actinomycete
MSDLTAVPTPESLRAWLVDCVADHLGRAPAGIATDVPLTTYGLDSVYALSIAAELEDHLDVSLDPTLIWDHPTIDALSAALVAELRSA

(19) United States
(12) Patent Application Publication
(10) Pub. No.: US 2003/0211567 A1
(43) Pub. Date: Nov. 13, 2003

(54) Compositions, methods and systems for discovery of lipopeptides

(75) Inventors: Chris M. Farnet, Outremont (CA); Alfredo Staffa, Saint-Laurent (CA); Emmanuel Zazopoulos, Montreal (CA)

Correspondence Address: PALMER & DODGE, LLP, KATHLEEN M. WILLIAMS, 111 HUNTINGTON AVENUE, BOSTON, MA, 02199, US

(73) Assignee: Ecopia Biosciences, Inc.

(21) Appl. No.: 10/329,027

(22) Filed: Dec. 24, 2002

Related U.S. Application Data
(63) Continuation-in-part of application No. 09/976,059, filed on Oct. 15, 2001.
(63) Continuation-in-part of application No. 10/232,370, filed on Sep. 3, 2002.
(63) Continuation-in-part of application No. 09/910,813, filed on Jul. 24, 2001.
(60) Provisional application No. 60/342,133, filed on Dec. 26, 2001.
(60) Provisional application No. 60/372,789, filed on Apr. 17, 2002.

(51) Int. Cl. C12P 21/06; C07H 21/04; C12N 9/10; C12N 1/21; C12N 15/74

(52) U.S. Cl. 435/68.1; 435/69.1; 435/193; 435/320.1; 435/252.3; 536/23.2

(57) ABSTRACT

The invention relates to isolated polypeptides involved in lipopeptide biosynthesis and polynucleotides encoding such polypeptides. In particular, the isolated polypeptide may be an acyl-specific C-domain, an adenylating enzyme, or an acyl carrier. The invention also relates to methods for detecting a polypeptide involved in lipopeptide biosynthesis or a polynucleotide encoding such a polypeptide, as well as relevant useful computer readable medium and computer systems.

### RELATED APPLICATIONS

This application claims the benefit of U.S. Provisional Application No. 60/342,133, filed on Dec. 26, 2001, U.S. Provisional Application No. 60/372,789, filed on Apr. 17, 2002. The present application is a continuation-in-part of U.S. application Ser. No. 09/976,059, filed Oct. 15, 2001, and of U.S. application Ser. No. 10/232,370, filed Sep. 3, 2002, which is a continuation-in-part of U.S. application Ser. No. 09/910,813 filed Jul. 24, 2001. The teachings of the above applications are incorporated herein by reference in their entirety.

### FIELD OF INVENTION

The invention relates to genes and proteins involved in the biosynthesis of lipopeptides and related compounds, and to methods, systems and compositions for the discovery and engineering of new lipopeptide biosynthetic loci and new lipopeptides.

### BACKGROUND

Lipopeptides are natural products that exhibit potent, broad-spectrum antibiotic activity with a high potential for biotechnological and pharmaceutical applications as antimicrobial, antifungal, or antiviral agents. Examples include compounds such as lichenysin, fengycin, surfactin, syringomycin, serrawettin, ramoplanin, daptomycin, A54145, the “calcium-dependent antibiotic” of Streptomyces coelicolor, echinocandin, pneumocandin, aculeacin, etc. Even within a group of relatively closely related actinomycete lipopeptide producers, lipopeptide natural products may differ in structure and can be classified into distinct sub-groups based on their chemical features. Lipoglycopeptides are lipopeptide natural products that are glycosylated, for example, ramoplanin. Acidic lipopeptides are lipopeptide natural products that are characterized by having acidic amino acid residues incorporated in the peptide chain portion of the lipopeptide, for example, daptomycin, A54145 and the calcium dependent antibiotic of Streptomyces coelicolor.

A single microorganism may produce a mixture of related lipopeptides that differ in the lipid moiety that is attached to the peptide core via a free amine, usually the N-terminal amine of the peptide core. The lipid moiety can have a major influence on the biological properties of lipopeptide natural products. For example, the lipopeptide antibiotic A21978C complex produced by S. roseosporus comprises at least six related microbiologically active factors C₀, C₁, C₂, C₃, C₄, and C₅ All factors of the lipopeptide antibiotic A21978C complex bear an identical 13-amino acid cyclic, acidic polypeptide core, but differ from one another in the identity of the fatty acyl group at the terminal amino group. The biological properties, e.g., antibacterial efficacy, toxicity, solubility, etc. of the different A21978C factors vary. One of the six factors identified as part of the A21978C complex, the A21978C factor C₀, is also known as daptomycin. Likewise, the A54145 antibiotics produced by S. fradiae are a group of lipopeptides related to the A21978C complex. Like the A21978C complex, the A54145 antibiotics comprise at least eight microbiologically active, related factors A, A₁, B, B₁, C, D, E, and F. Each A54145 factor bears a cyclic 13-amino acid, acidic polypeptide core and a fatty acyl group attached to the N-terminal amine. The eight A54145 factors differ in the identity of the amino acid residue at positions 12 and 13 of the peptide core as well as in the identity of the fatty acyl group attached to the terminal amino group of the amino acid residue at position 1. There is a continuing need for compositions, methods and systems useful in discovery of lipopeptide natural products and related compounds.

Methods for natural product discovery have faced many challenges. Discovery efforts that focus on microbial-derived natural products are hampered by difficulties in cultivating the microbes; indeed most microbes have yet to be cultivated in vitro. In addition, many cultivated microorganisms are not amenable to fermentation. Furthermore many secondary metabolites are not expressed to detectable levels under in vitro conditions. Furthermore, natural products produced under in vitro conditions often vary according to the growth conditions, e.g. nutrients provided, and may not be representative of the full biosynthetic potential of the microorganism. Genomics-based compositions, methods and systems for discovering lipopeptides would obviate or mitigate one or more of these disadvantages.

Lipopeptides produced by micororganisms are synthesized nonribosomally on large multifunctional proteins termed nonribosomal peptide synthetases (NRPSs) (Doekel and Marahiel, 2001, Metabolic Engineering, Vol. 3, pp. 64-77). NRPSs are modular proteins that consist of one or more polyfunctional polypeptides each of which is made up of modules. The amino-terminal to carboxy-terminal order and specificities of the individual modules correspond to the sequential order and identity of the amino acid residues of the peptide product. Each NRPS module recognizes a specific amino acid substrate and catalyzes a stepwise condensation to form the growing peptide chain. The identity of the amino acid recognized by a particular unit can be determined by comparison with other units of known specificity (Challis and Ravel, 2000, FEMS Microbiology Letters, Vol. 187, pp. 111-114). In many peptide synthetases, there is a strict correlation between the order of repeated units in a peptide synthetase and the order in which the respective amino acids appear in the peptide product, making it possible to correlate peptides of known structure with putative genes encoding their synthesis, as demonstrated by the identification of the mycobactin biosynthetic gene cluster from the genome of Mycobacterium tuberculosis (Quadri et al., 1998, Chem. Biol. Vol. 5, pp. 631-645).

The modules of a peptide synthetase are composed of smaller units or “domains” that each carry out a specific role in the recognition, activation, modification and joining of amino acid precursors to form the peptide product. One type of domain, the adenylation (A) domain, is responsible for selectively recognizing and activating the amino acid that is to be incorporated by a particular unit of the peptide synthetase. This activation step is ATP-dependent and involves the transient formation of an amino-acyl-adenylate. The activated amino acid is covalently attached to the peptide synthetase through another type of domain, the thiolation (T) domain, that is generally located adjacent to the A domain. The T domain is post-translationally modified by the covalent attachment of a phosphopantetheinyl prosthetic arm to a conserved serine residue. The activated amino acid substrates are tethered onto the nonribosomal peptide synthetase via a thioester bond to the phosphopantetheinyl prosthetic arm of the respective T domains. Amino acids joined to successive units of the peptide synthetase are subsequently covalently linked together by the formation of amide bonds catalyzed by another type of domain, the condensation (C) domain.

Little is known about the mechanism involved in attachment of lipid moieties to the peptide core. The literature is sparse regarding the enzymatic mechanism or timing of addition of the acyl group to lipopeptide natural products. In particular, the enzymes involved in N-acylation of peptide natural products have not been identified, and it remains unknown whether acylation occurs prior to, concomitant with, or subsequent to the formation of the peptide core. Doekel and Marahiel, (2001, Metabolic Engineering, 3, 64-77) reviews catalytic domains in peptide synthetases and notes that condensation domain sequences vary according to the domain arrangements of NRPSs, referring to condensation domains located C-terminal to epimerization domains, condensation domains located C-terminal to thiolation domains, and condensation domains involved in initiation of acyl-transfer during assembly of lipopeptides. Understanding the mechanism by which the lipid moieties are covalently attached to the peptide core would allow for introduction of alternative fatty acyl moieties onto a given peptide core by means of recombinant DNA technologies, or to increase the yield of product(s) containing the desirable fatty acyl moiety or moieties by recombinant DNA technologies.

Selective feeding experiments indicate that growth nutrients can affect the relative amounts of lipopeptide products. Growth conditions that favor the synthesis of one given lipid precursor will preferentially lead to the synthesis of the corresponding lipopeptide containing that lipid moiety. For example, daptomycin is normally produced by S. roseosporus in trace amounts. A great deal of effort is required to generate adequate amounts of biologically pure daptomycin. Continuous feeding of fermentation cultures with caproic acid or decanoic acid mixed 1:1 (v:v) in methyl oleate has been shown to increase the yield of daptomycin (R. H. Baltz, Lipopeptide Antibiotics Produced by Streptomyces roseosporus and Streptomyces fradiae, in: Biotechnology of Antibiotics, Second Edition, pp. 415-435, edited by W. R. Strohl). Alternatively, a chemical process requiring enzymatic deacylation of A21978C factors, protection of a certain reactive sidechain in the peptide portion of the compound, synthetic addition of the fatty acyl group, and finally deprotection to yield the desired daptomycin product has been developed. However, these methods are compound-specific, laborious and inefficient and highlight the need for improved methods of producing lipopeptides and derivatives thereof.

### SUMMARY OF THE INVENTION

In one aspect, the invention provides an isolated polynucleotide encoding an acyl-specific C-domain, wherein said isolated polynucleotide encodes a polypeptide which comprises at least 45% sequence identity to at least one sequence selected from SEQ ID NOS: 1 and 2. Certain embodiments expressly exclude one or more sequences, in particular the nucleotide sequence corresponding to the C-domain of NRPS protein of GenBank accession no. CAB 38518, i.e. coordinates 195135 to 217526 of Genbank nucleotide accession AL939115, and SEQ ID NO: 21. Other embodiments, exclude nucleic acid sequences originating from an organism other than an organism of the actinomycetes taxon. Other sequences can be excluded without departing from the scope of the invention. In a related aspect the invention provides an isolated polynucleotide comprising a sequence selected from the group consisting of: (a) a sequence selected from the group consisting of SEQ ID NOS: 5, 7, 9, 11,13,15, 17 and 19; (b) a sequence that is complementary to (a); (c) a sequence which hybridizes to said sequence of (a) or (b) under conditions of high stringency; and (d) a sequence which has at least 70% or higher homology to said sequence of (a), (b), or (c). Certain embodiments expressly exclude one or more sequences, in particular the nucleotide sequence corresponding to the C-domain of NRPS protein of GenBank accession no. CAB 38518, i.e. coordinates 195135 to 217526 of Genbank nucleotide accession AL939115, and SEQ ID NO: 21. Other embodiments, exclude nucleic acid sequences originating from an organism other than an organism of the actinomycetes taxon. Other sequences can be excluded without departing from the scope of the invention. In one embodiment of the invention, the acyl-specific C-domain encoded by the isolated polynucleotide is involved in lipopeptide acyl-capping. In one embodiment the acyl-specific C-domains reside in cosmids 008CH, 184CM and 024CK having accession numbers IDAC 190901-2, IDAC 260202-1 and IDAC 260202-5, respectively.

In a further embodiment, the isolated polynucleotide encoding an acyl-specific C-domain resides in a gene locus selected from the group consisting of the biosynthetic locus for ramoplanin from Actinoplanes sp. ATCC 33076; the biosynthetic locus for A21978C from Streptomyces roseosporus NRRL 11379; the biosynthetic locus for A54145 from Streptomyces fradiae ATCC 18158; the biosynthetic locus for the calcium-dependent antibiotic from Streptomyces coelicolor A3(2); the biosynthetic locus for a lipopeptide natural product from Streptomyces ghanaensis NRRL B-12104; the biosynthetic locus for a lipopeptide natural product from Streptomyces refuineus NRRL 3143; the biosynthetic locus for a lipopeptide natural product from Streptomyces aizunensis NRRL B-11277; the biosynthetic locus for a lipopeptide natural product from Actinoplanes nipponensis FD 24834 ATCC 31145; and the biosynthetic locus for a lipopeptide natural product from a Streptomyces sp. organism.

In another embodiment, the isolated polynucleotide encoding an acyl-specific C-domain does not reside in the biosynthetic locus for the calcium-dependent antibiotic from Streptomyces coelicolor A3(2) (CADA).

The invention provides two or more isolated polynucleotides, wherein the first polynucleotide encodes a polypeptide which comprises at least 45% sequence identity to at least one sequence selected from SEQ ID NOS: 1 and 2, and the second polynucleotide encodes a polypeptide selected from the group consisting of a polypeptide having at least 55% sequence identity to SEQ ID NO: 3 and a polypeptide having at least 50% sequence identity to SEQ ID NO: 4. In a related aspect the invention provides two or more isolated polynucleotides wherein the first polynucleotide encodes an acyl-specific C-domain and the second polynucleotide encodes an adenylating enzyme, an acyl carrier protein or a fusion of an adenylating enzyme and an acyl carrier protein.

The invention also provides an isolated polynucleotide comprising a sequence selected from the group consisting of: (a) a sequence selected from the group consisting of SEQ ID NOs. 23, 25, 27, 29, 31, 33, 35, 37, 39, 41, 43, 45 and 47; (b) a sequence that is complementary to (a); (c) a sequence which hybridizes to said sequence of (a) or (b) under conditions of high stringency; and (d) a sequence which has at least 70% or higher homology to said sequence of (a), (b), or (c). In one embodiment the polynucleotide encodes a polypeptide selected from the group consisting of a polypeptide having at least 55% sequence identity to SEQ ID NO: 3. In another embodiment, the polynucleotide encodes a polypeptide having at least 50% sequence identity to SEQ ID NO: 4.

In one embodiment the polynucletide encodes an adenylating enzyme. In another embodiment the polynucleotide encodes an acyl carrier protein. In a further embodiment, the polynucleotide encodes a fusion of an adenylating enzyme and an acyl carrier protein. In another embodiment the polypeptide encoding an adenylating enzyme, an acyl carrier protein or a fusion of the two is from a biosynthetic locus selected from the group consisting of the biosynthetic locus for ramoplanin from Actinoplanes sp. ATCC 33076; the biosynthetic locus for A21978C from Streptomyces roseosporus NRRL 11379; the biosynthetic locus for A54145 from Streptomyces fradiae ATCC 18158; the biosynthetic locus for a lipopeptide natural product from Streptomyces ghanaensis NRRL B-12104; the biosynthetic locus for a lipopeptide natural product from Streptomyces refuineus NRRL 3143; the biosynthetic locus for a lipopeptide natural product from Streptomyces aizunensis NRRL B-11277; the biosynthetic locus for a lipopeptide natural product from Actinoplanes nipponensis FD 24834 ATCC 31145; and the biosynthetic locus for a lipopeptide natural product from a Streptomyces sp. organism. In one embodiment the adenylating enzyme is from cosmids 008CO and 024CK having accession numbers IDAC 190901-2 and IDAC 260202-5, respectively. In another embodiment the acyl carrier protein is from cosmids 008CH and 024CK having accession numbers IDAC 190901-3 and IDAC 260202-5 respectively. In one embodiment the fusion protein containing an adenylating enzyme and an acyl carrier protein is from cosmid 184CM having accession number IDAC 260202-1.

The invention also provides an isolated acyl-specific C-domain comprising at least 45% sequence homology to at least one sequence selected from SEQ ID NO. 1 and SEQ ID NO. 2. Certain embodiments expressly exclude one or more sequences, in particular the polypeptide sequence corresponding to the C-domain of NRPS protein of GenBank accession no. CAB 38518, and SEQ ID NO: 22. Other embodiments, exclude polypeptide sequences originating from an organism other than an organism of the actinomycetes taxon. Other sequences can be excluded without departing from the scope of the invention. In a related aspect, the invention provides an isolated acyl-specific C-domain comprising a polypeptide sequence selected from the group consisting of: (a) a sequence selected from the group consisting of SEQ ID NOs. 6, 8, 10,12, 14,16,18, 20 and 22; and (b) a sequence which has at least 70% or higher homology to said sequence of (a). Certain embodiments expressly exclude one or more sequences, in particular the polypeptide sequence corresponding to the C-domain of NRPS protein of GenBank accession no. CAB 38518, and SEQ ID NO: 22. Other embodiments, exclude polypeptide sequences originating from an organism other than an organism of the actinomycetes taxon. Other sequences can be excluded without departing from the scope of the invention.

The invention further provides two or more isolated polypeptides, wherein the first isolated polypeptide is an acyl-specific C-domain comprising at least 45% sequence homology to at least one sequence selected from SEQ ID NO. 1 and SEQ ID NO. 2, and the second isolated polypeptide is selected from the group consisting of a polypeptide having at least 55% identity to SEQ ID NO. 3 and a polypeptide having at least 50% identity to SEQ ID NO. 4. In still a further aspect, the invention provides an N-acyl-capping cassette comprising at least one acyl-specific C-domain polypeptide and another polypeptide selected from the group consisting of an adenylating protein and an acyl-carrier protein.

In one embodiment, the isolated acyl-specific C-domain is not from the biosynthetic locus for the calcium-dependent antibiotic from Streptomyces coelicolor A3(2) (CADA).

The invention provides an isolated polypeptide comprising a polypeptide selected from the group consisting of: (a) SEQ ID NOs. 24, 26, 28, 30, 32, 34, 36, 38, 40, 42, 44, 46 and 48; and (b) a sequence which has at least 70% or higher homology to said sequence of (a). In one embodiment, such isolated polypeptide is not from the biosynthetic locus for the calcium-dependent antibiotic from Streptomyces coelicolor A3(2) (CADA).

The invention further provides a computer readable medium comprising a computer program and data, comprising: (a) a computer program stored on said media containing instructions sufficient to implement a process for effecting the identification, analysis, or modeling of a representation of a polynucleotide or polypeptide sequence; (b) data stored on said media representing a sequence of a polynucleotide selected from the group consisting of: (i) a polynucleotide encoding an acyl-specific C-domain, said polynucleotide encoding a polypeptide having at least 45% sequence identity with either SEQ ID NO: 1 or SEQ ID NO: 2; (ii) a polynucleotide encoding a polypeptide having at least 55% sequence identity with SEQ ID NO: 3; and (iii) a polynucleotide encoding a polypeptide having at least 50% sequence identity with SEQ ID NO: 4; and (c) a data structure reflecting the underlying organization and structure of said data to facilitate said computer program access to data elements corresponding to logical sub-components of the sequence, said data structure being inherent in said program and in the way in which said computer program organizes and accesses said data. In a related aspect, the invention provides a computer readable medium comprising a computer program and data, comprising: (a) a computer program stored on said media containing instructions sufficient to implement a process for effecting the identification, analysis, or modeling of a representation of a polypeptide sequence; (b) data stored on said media representing a sequence of a polypeptide selected from the group consisting of: (i) a polypeptide representing an acyl-specific C-domain and having at least 45% sequence identity with either SEQ ID NO: 1 or SEQ ID NO: 2; (ii) a polypeptide having at least 55% sequence identity with SEQ ID NO: 3; and (iii) a polypeptide having at least 50% sequence identity with SEQ ID NO: 4; and (c) a data structure reflecting the underlying organization and structure of said data to facilitate said computer program access to data elements corresponding to logical sub-components of the sequence, said data structure being inherent in said program and in the way in which said computer program organizes and accesses said data.

The invention also provides a memory for storing data that can be accessed by a computer programmed to implement a process for effecting the identification, analysis, or modeling of a sequence of a polynucleotide or a polypeptide, said memory comprising data representing a polynucleotide selected from the group consisting of: (a) a polynucleotide encoding an acyl-specific C-domain, said polynucleotide encoding a polypeptide having at least 45% sequence identity with either SEQ ID NO: 1 or SEQ ID NO: 2; (b) a polynucleotide encoding a polypeptide having at least 55% sequence identity with SEQ ID NO: 3; and (c) a polynucleotide encoding a polypeptide having at least 50% sequence identity with SEQ ID NO: 4. In a related aspect, the invention provides a memory for storing data that can be accessed by a computer programmed to implement a process for effecting the identification, analysis, or modeling of a sequence of a polypeptide, said memory comprising data representing a polypeptide selected from the group consisting of: (a) a polypeptide having at least 45% sequence identity with either SEQ ID NO: 1 or SEQ ID NO: 2; (b) a polypeptide having at least 55% sequence identity with SEQ ID NO: 3; and (c) a polypeptide having at least 50% sequence identity with SEQ ID NO: 4.

The invention provides a method for detecting a polypeptide involved in lipopeptide biosynthesis or a polynucleotide encoding such a polypeptide comprising the step of identifying (a) a polypeptide having at least 45% sequence identity to SEQ ID NO: 1 or SEQ ID NO: 2, or (b) a polynucleotide encoding a polypeptide having at least 45% sequence identity to SEQ ID NO: 1 or SEQ ID NO: 2, wherein said at least 45% sequence identity indicates a polypeptide involved in lipopeptide biosynthesis. In one embodiment the method comprises the steps of: (a) providing a reference polynucleotide or polypeptide sequence selected from the group consisting of a polynucleotide or polypeptide sequences representing an acyl-specific domain; (b) comparing said reference sequence to one or more candidate polynucleotide or polypeptide sequences stored on a computer readable medium; (c) determining level of homology between said reference sequence and said one or more candidate sequences, and (d) identifying a candidate sequence which shares at least 70% homology with reference sequence. In one embodiment the method further comprising the step of identifying, in proximity to the polypeptide of (a) or the polynucleotide of (b), at least (c) one polypeptide having at least 55% sequence identity to SEQ ID NO: 3 or one polynucleotide sequence encoding a polypeptide having at least 55% sequence identity to SEQ ID NO: 3; or (d) one polypeptide having at least 50% sequence identity to SEQ ID NO: 4 or one polynucleotide sequence encoding a polypeptide having at least 50% sequence identity to SEQ ID NO: 4. In another embodiment of the method the polypeptide of c) is a polypeptide of SEQ ID NO: 24, 26, 28, 30, 32, 34, 36, 38 or 40, or a polypeptide having at least 70% sequence identity to a polypeptide of SEQ ID NO: 24, 26, 28, 30, 32, 34, 36, 38 or 40; or the nucleotide of (d) is a nucleotide encoding a polypeptide of SEQ ID NO: 24, 26, 28, 30, 32, 34, 36, 38 or 40 or a nucleotide encoding a polypeptide having at least 70% sequence identity to a polypeptide of SEQ ID NO: 24, 26,28,30, 32, 34, 36, 38 or 40.

The invention provides a computer system comprising: (a) a database of reference sequences, wherein the reference sequences encode proteins involved in lipid biosynthesis, and wherein the reference sequences include one or more of: (i) a polypeptide sequence representing an acyl-specific C-domain or a polynucleotide encoding an acyl-specific C-domain; and (b) a user interface capable of: (ii) receiving a test sequence for comparing against each of the reference sequences in the database; and (iii) displaying the results of the comparison. In one embodiment, reference sequences of the computer system further include one or more of: (iv) a polypeptide sequence representing an adenylating enzyme or a polynucleotide encoding an adenylating enzyme; and (v) a polypeptide sequence representing an acyl carrier protein or a poynucleotide encoding an acyl carrier protein. In another embodiment, the reference sequence of (i) is selected from SEQ ID NOS: 1, 2, 5, 6, 7, 8, 9, 10, 11, 12, 13, 14, 15, 16, 17, 18, 19, 20, 21 and 22; the reference sequence of (iv) is selected from SEQ ID NOS: 3, 23, 24, 25, 26, 27, 28, 29, 30, 31, 32, 33 and 34; and the reference sequence of (v) is selected from SEQ ID NO: 4, 37, 38, 39, 40, 41, 42, 43, 44, 45, 46, 47 and 48.

### BRIEF DESCRIPTION OF THE DRAWINGS

FIGS. 1a, 1b, 1c, 1d and 1e represent schematic views of the biosynthetic loci for: (1a) ramoplanin from Actinoplanes sp. ATCC 33076 (RAMO) and A21978C from Streptomyces roseosporus NRRL 11379 (DAPT); (1b) A54145 from Streptomyces fradiae ATCC 18158 (A541) and the lipopeptide from Streptomyces ghanaensis NRRL B-12104 (009H); (1c) a lipopeptide from Streptomyces refuineus NRRL 3143 (024A) and a lipopeptide from Streptomyces aizunensis NRRL B-1 1277 (023C); (1d) a lipopeptide from Actinoplanes nipponensis FD 24834 ATCC 31145 (A410) and a putative lipopeptide natural product (070B) from organism 070 in Ecopia's private culture collection; and (1e) the calcium-dependent antibiotic from Streptomyces coelicolor A3 (CADA), showing a scale in base pairs, and the relative position and orientation of open reading frames (ORFs) encoding representative acyl-specific C-domains of the invention and representative adenylating enzymes and acyl carrier proteins of the invention. Deposited cosmids containing genes of the invention are also indicated in regard to RAMO, A541 and 024A.

FIG. 2 represents a dendrogram showing the evolutionary relatedness of C-domains from various lipopeptide NRPSs with a clearly branching cluster of representative C-domains of the invention involved in N-acylation highlighted in gray.

FIGS. 3a and 3b represent an amino acid alignment of representative acyl-specific C-domains of the invention as found in each of the RAMO, DAPT, A541, CADA, 009H, 024A, 023C, A410 and 070B lipopeptide biosynthetic loci. Conserved motifs are highlighted. In each of the clustal alignments a line above the alignement is used to mark strongly conserved positions. In addition, three characters, namely ★(asterisk),: (colon) and . (period) are used, wherein “★” indicates positions which have a single, fully conserved residue; “:” indicates that one of the following strong groups is fully conserved: STA, NEQK, NHQK, NDEQ, QHRK, MILV, MILF, HY, and FYW; and “.” Indicates that one of the following weaker groups is fully conserved: CSA, ATV, SAG, STNK, STPA, SGND, SNDEQK, NDEQHK, NEQHRK, FVLIM, and HFY.

FIGS. 4a, 4b and 4c represent an amino acid alignment of representative ADLE proteins of the invention as found in each of the RAMO, DAPT, 009H, 024A, 023C and A410 loci, together with the ADLE portion of the ADLF fusion protein from the A541 locus. Conserved motifs of acyl CoA ligases are indicated by lines on top of the sequences.

FIG. 5 is an amino acid alignment of representative ACPH proteins of the invention from the RAMO, DAPT, 009H, 024A, 023C and A410 loci together with the corresponding portion of the ADLF fusion protein from the A541 locus. The conserved serine residue of the thiolation domain to which a phosphopantetheine group is covalently attached post-translationally is highlighted.

FIG. 6a is a dendrogram showing the evolutionary relatedness of the representative NRPS C-domains of the invention. FIG. 6b is a dendrogram showing the evolutionary relatedness of the representative ADLE proteins of the invention. FIG. 6c is a dendrogram showing the evolutionary relatedness of the representative ACPH proteins of the invention.

FIGS. 7a and 7b illustrate a general biosynthetic scheme for formation of N-acyl peptide linkage in lipopeptides using the acyl-specific C-domain, ADLE protein and ACPH protein of the invention.

FIG. 8 illustrates the biosynthetic scheme of FIGS. 7a and 7b as applied to formation of the N-acyl peptide linkage in ramoplanin and A54145.

FIGS. 9a and 9b are photographs of plates generated in the bioassay of anionic lipopeptide isolation experiments and illustrating an enrichment of activity, based on IRA67 anion exchange chromatography of lipopeptides from Streptomyces refuineus subsp. thermotolerans and Streptomyces fradiae.

FIG. 10a and 10b illustrate use of NRPS biosynthetic machinery of a nonlipopeptide natural product, complestatin, to produce an N-acylated analogue of complestatin. FIG. 10a illustrates the biosynthesis of complestatin. FIG. 10b illustrates a rationally designed recombinant NRPS system that gives rise to N-acylated complestatin analogue(s).

FIG. 11 is a block diagram of a computer system according to one embodiment of the invention.

FIG. 12 is a flow chart representing a process performed by the computer system to compare candidate sequences with one or more reference sequences according to one embodiment of the invention.

FIG. 13 is a flow chart representing a process performed by the computer system to compare candidate sequences with one or more reference sequences and the display of comparison results according to one embodiment of the invention.

FIG. 14 is a chart the domains disclosed according to some embodiments of the invention.

### DETAILED DESCRIPTION

The invention provides compositions, methods and systems useful in the discovery and engineering of lipopeptides and related compounds. The compositions can be used in identifying lipopeptide natural products, lipopeptide genes, lipopeptide gene clusters and lipopeptide-producing organisms.

Lipopeptide biosynthetic loci from a variety of organisms were discovered and analyzed. For convenience, the lipopeptide biosynthetic loci and the organism in which the locus is found is sometimes indicated by reference to a source designation wherein “RAMO” refers to the biosynthetic locus for ramoplanin from Actinoplanes sp. ATCC 33076, “DAPT” refers to the biosynthetic locus for A21978C from Streptomyces roseosporus NRRL 11379, “A541” refers to the biosynthetic locus for A54145 from Streptomyces fradiae ATCC 18158, “CADA” refers to the biosynthetic locus for the calcium-dependent antibiotic from Streptomyces coelicolor A3(2) (Bentley et al., 2002, Nature, vol. 417, pp 141-147), “009H” refers to the biosynthetic locus for a lipopeptide natural product from Streptomyces ghanaensis NRRL B-12104, “024A” refers to the biosynthetic locus for a lipopeptide natural product from Streptomyces refuineus NRRL 3143, “023C” refers to a biosynthetic locus for a lipopeptide natural product from Streptomyces aizunensis NRRL B-1 1277, “A410” refers to the biosynthetic locus for a lipopeptide natural product from Actinoplanes nipponensis FD 24834 ATCC 31145, and “070B” refers to the biosynthetic locus for a lipopeptide natural product from a Streptomyces sp. organism in Ecopia's private culture collection.

Surprisingly, a conserved gene domain and conserved genes common to lipopeptide biosynthetic loci have been discovered. The conserved domain is referred to as an “acyl-specific C-domain (unusual C-domain)” which means a condensation-domain (C-domain) involved in N-acyl capping for lipopeptide biosynthesis. The “acyl-specific C-domain” is required for the N-acyl peptide linkage found in lipopeptides between the lipid moiety and the first amino acid residue of the peptide core. Representative examples of the acyl specific C-domains of the invention include the acyl specific C-domain residing in the ramoplanin biosynthetic locus from Actinoplanes sp. ATCC 33076 (SEQ ID NO: 6), the acyl-specific C-domain residing in the A21978C locus in Streptomyces roseosporus NRRL 11379 (SEQ ID NO: 8), the acyl specific C-domain residing in the A54145 locus in Streptomyces fradiae ATCC 18158 (SEQ ID NO: 10), the acyl-specific C-domain residing in a lipopeptide biosynthetic locus in Streptomyces ghanaensis NRRL B-12104 (SEQ ID NO: 12), the acyl-specific C-domain residing in a lipopeptide biosynthetic locus in Streptomyces refuineus NRRL 3143 (SEQ ID NO: 14), the acyl-specific C-domain residing in a lipopeptide biosynthetic locus in Streptomyces aizunensis NRRL B-11277 (SEQ ID NO: 16), the acyl-specific C-domain residing in the A41,012 lipopeptide biosynthetic locus in Actinoplanes nipponensis FD 24834 ATCC 31145 (SEQ ID NO: 18), the acyl-specific C-domain residing in a putative lipopeptide biosynthetic locus from the Streptomyces sp. organism 070 in Ecopia's private culture collection (SEQ ID NO: 20) and the acyl-specific C-domain residing in the biosynthetic locus for the calcium-dependent antibiotic from the Streptomyces coelicolor A3(2) (SEQ ID NO: 22). Certain embodiments expressly exclude the acyl-specific C-domain residing in the calcium dependent antibiotic biosynthetic locus from the Streptomyces coelicolor A3(2) (SEQ ID NO: 22 and the polypeptide sequence corresponding to the C-domain of NRPS protein of GenBank accession no. CAB 38518). Other embodiments, exclude polypeptide sequences originating from an organism other than an organism of the actinomycetes taxon.

An “acyl-specific C-domain” of the present invention is defined structurally as a polypeptide sequence that produces an alignment with at least 45% identity to one of the two following consensus sequences using the BLASTP 2.0.10 algorithm (with the filter option -F set to false, the gap opening penalty -G set to 11, the gap extension penalty -E set to 1, and all remaining options set to default values):

>Consensus sequence 1
GgIReLmAgQLAvWhAqQLaPenPvYnvGEYveidGevDIdLLvaAvrrv
meEadaaRLRfrevDgvPRQYfaedeDypveViDvSaeaDPrAAAeSIMa
aDLrRprDlrdgeLytqkiykvgedlvfWYqRahHiiIDGrSaGIVaSRV
AaVYsALaaGgdveegALPsssVLmdAedeYraSeefeIDReYWreaLAg
IPeevslganePsrlprepvRheedvsdaaAaeLraaARRLgTSIAgIai
AAAAIYqHrITGqrDVvvgVPVaGRsktaeIdiPGMTaNVvPvRIAVaPk
ttVaeLvrqvaRGVrdGLRHQRYrYediIdDIkLvgrdgLypIIVNvISf
DydLrFGdAvsvahgLSagpvddvsIdvYdrsSdGsmkvvvdvNPDItdr
sdadEvarkFIaIIrWLaesdAeepVaridLIded
>Consensus sequence 2
svRhgvtaAQrgvWvAQQLrpdsrIYnCGIyLeIdgaIDpavLsrAvRrt
IaeTEALRsrFeedddGaIIqrvIapaPdeqtrIIeDGvPYtPvLLRHiD
IsgddDPeaAArrWMDadIAePvdLdragtsrHaLItLGgdRhLIYIgYH
HiaLDGfGaaLYIdRIAaVYrALrtGrePppcpFgpLdrIvaeeaaYrdS
aRhrrDrayWtgrfadIpEPvgLagraAaAapapLRrtvrLpperTaaLa
aaAeatGsrWpavviAAVAAFIrRIagaeeVVvgLPVTARvTrAAIrTPG
MLaNvIPLRLeVrqgasfAaLIeetsraIsaILRHQRFRGEdLgReLGIa
GerAgIapttVNVMaFapvIdFGdcrAvvHqLSsGPVeDLaInIyGTPgt
GdeIrvtvaANPalYtaddVaslqeRLvRfLaalgaDPaapvGrvrLLdp
a

where consensus sequence 1 is based on the sequences of the acyl-specific condensation domains from the calcium-dependent antibiotic (CADA) locus in Streptomyces coelicolor A3(2) (GenBank accession numbers CAB38517, CAB38518; CAB38516 and CAB38876), A21978C (DAPT) locus in Streptomyces roseosporus (NRRL 11379), A54145 locus in Streptomyces fradiae (ATCC 18158), A410 locus from an Actinoplanes nipponensis, 009H locus from Streptomyces ghanaensis (NRRL B-12104), and 024A locus in Streptomyces refuineus (NRRL 3143); and where consensus sequence 2 is based on the sequences of the acyl specific condensation domains from the ramoplanin (RAMO) locus (Actinoplanes sp. ATCC 33076), 023C locus from Streptomyces aizunensis (NRRL B-11277), and 070B, a putative lipopeptide locus found from Ecopia's private culture collection.

The consensus sequences were generated as follows. First, the listed sequences were aligned with the ClustaIX 1.81 program using default settings. Then a profile hidden Markov model (HMM) was made from the alignment file with the hmmbuild program of the HMMER 2.2 package (Sean Eddy, Washington University; world-wide-web hmmer.wustl.edu/) and was calibrated with the hmmcalibrate program of the HMMER package, both using default settings. Briefly, a profile hidden Markov model is a statistical description of a sequence family's consensus. HMMER is a freely distributable implementation of profile HMM software for protein sequence analysis and is available from the above web site. Finally, the consensus sequences were generated from the HMM with the hmmemit program of the HMMER package using the -c option so as to predict a single majority rule consensus sequence from the HMM's probability distribution. Highly conserved amino acid residues (p>=0.5) are shown in upper case in the consensus sequence, others are shown in lower case.

A “polynucleotide encoding an acyl-specific condensation domain (C-domain)” refers to a polynucleotide encoding an acyl-specific C-domain. Representative examples of a polynucleotide encoding an acyl specific C-domain of the invention include the polynucleotide encoding the acyl specific C-domain residing in the ramoplanin biosynthetic locus from Actinoplanes sp. ATCC 33076 (SEQ ID NO: 5), the polynucleotide encoding the acyl-specific C-domain residing in the A21978C locus in Streptomyces roseosporus NRRL 11379 (SEQ ID NO: 7), the polynucleotide encoding the acyl specific C-domain residing in the A54145 locus in Streptomyces fradiae ATCC 18158 (SEQ ID NO: 9), the polynucleotide encoding the acyl-specific C-domain residing in a lipopeptide biosynthetic locus in Streptomyces ghanaensis NRRL B-12104 (SEQ ID NO: 11), the polynucleotide encoding the acyl-specific C-domain residing in a lipopeptide biosynthetic locus in Streptomyces refuineus NRRL 3143 (SEQ ID NO: 13), the polynucleotide encoding the acyl-specific C-domain residing in a lipopeptide biosynthetic locus in Streptomyces aizunensis NRRL B-11277 (SEQ ID NO: 15), the polynucleotide encoding the acyl-specific C-domain residing in a lipopeptide biosynthetic locus in Actinoplanes nipponensis FD 24834 ATCC 31145 (SEQ ID NO: 17), the polynucleotide encoding the acyl-specific C-domain residing in a biosynthetic locus of a Streptomyces sp. in Ecopia's private culture collection (SEQ ID NO: 19), and the polynucleotide encoding the acyl-specific C-domain residing in the calcium dependent antibiotic biosynthetic locus from the Streptomyces coelicolor A3(2) (SEQ ID NO: 21). Certain embodiments expressly exclude polynucleotides encoding the acyl-specific C-domain residing in the calcium dependent antibiotic biosynthetic locus from the Streptomyces coelicolor A3(2) (SEQ ID NO: 21 and nucleotide sequences encoding the polypeptide sequence of the C-domain of NRPS protein of GenBank accession no. CAB 38518, i.e. coordinates 195135 to 217526 of nucleotide accession AL939115 represent the nucleotide sequence of the NRPS of CAB38518). Other embodiments, exclude polypeptide sequences originating from an organism other than an organism of the actinomycetes taxon.

The acyl-specific C-domains of SEQ ID NOS: 6, 8, 10,12,14, 16,18 and 20 were compared using the BLASTP algorithm with the default parameters to the sequences of the National Center for Biotechnology Information (NCBI) nonredundant protein database and to sequences of the DECIPHER® database of microbial genes, pathways and natural products (Ecopia BioSciences Inc., St-Laurent, Canada). The accession numbers of the top GenBank hits of this BLAST analysis are presented in Table 1 along with the corresponding E values. The E value assists in the determination of whether two sequences display sufficient similarity to justify an inference of homology. The E value relates the expected number of chance alignments with an alignment score at least equal to the observed alignment score. An E value of 0.00 indicates a perfect homolog. The E-values are calculated as described in Altschul et al. 1990, J. Mol. Biol. 215(3):403-410; Gish et al., 1993, Nature Genetics 3:266-272.

TABLE 1

| | | | | | | | | |
|---|---|---|---|---|---|---|---|---|
| | | | | GenBank | prob- | % | % | proposed function of GenBank |
| SEQ id | Family | Locus | #aa | homology | ability | identity | similarity | match |

| | | | | | | | | |
|---|---|---|---|---|---|---|---|---|
| 6 | NRPS- | RAMO | 435aa | AAD56240. | 6e−46 | 111/391 | 167/391 | DhbF, Bacillus subtilis |
| | Cdom | | | 1,2378aa | | (28.39%) | (42.71%) | |
| | | | | BAB69380. | 1e−44 | 126/436 | 177/436 | non-ribosomal peptide |
| | | | | 1,1440aa | | (28.9%) | (40.6%) | synthetase,Streptomyces avermitilis |
| | | | | T30288, | 3e−42 | 128/429 | 170/429 | pristinamycin I synthase 2,Streptomyces |
| | | | | 2591aa | | (29.84%) | (39.63%) | pristinaespiralis |
| 8 | NRPS- | DAPT | 435aa | Np_627443. | 6e−64 | 160/451 | 214/451 | CDA peptide synthetase I,Streptomyces |
| | Cdom | | | 1,7463aa | | (35.48%) | (47.45%) | coelicolor |
| | | | | AAD56240. | 2e−57 | 133/430 | 199/430 | DhbF,Bacillus subtilis |
| | | | | 1,2378aa | | (30.93%) | (46.28%) | |
| | | | | T14591, | 3e−57 | 146/432 | 196/432 | actinomycin synthetase II,Streptomyces |
| | | | | 2611aa | | (33.8%) | (45.37%) | chrysomallus |
| 10 | NRPS- | A541 | 453aa | Np_627443. | 9e−48 | 150/462 | 187/462 | CDA peptide synthetase I,Streptomyces |
| | Cdom | | | 1,7463aa | | (32.47%) | (40.48%) | coelicolor |
| | | | | AAD56240. | 1e−45 | 131/447 | 190/447 | DhbF,Bacillus subtilis |
| | | | | 1,2378aa | | (29.31%) | (42.51%) | |
| | | | | T14591, | 5e−40 | 138/452 | 186/452 | actinomycin synthetase II,Streptomyces |
| | | | | 2611aa | | (30.53%) | (41.15%) | chrysomallus |
| 12 | NRPS- | 009H | 432aa | NP_627443. | 1e−53 | 148/452 | 195/452 | CDA peptide synthetase I,Streptomyces |
| | Cdom | | | 1,17463aa | | (32.74%) | (43.14%) | coelicolor |
| | | | | BAB69380. | 1e−53 | 140/431 | 198/431 | non-ribosomal peptide |
| | | | | 1,1440aa | | (32.48%) | (45.94%) | synthetase,Streptomyces avermitilis |
| | | | | AAD56240. | 8e−53 | 133/434 | 201/434 | DhbF,Bacillus subtilis |
| | | | | 1,2378aa | | (30.65%) | (46.31%) | |
| | NRPS- | 024A | 438aa | NP_627443. | 4e−53 | 143/414 | 190/414 | CDA peptide synthetase I,Streptomyces |
| | Cdom | | | 1,17463aa | | (34.54%) | (45.89%) | coelicolor |
| | | | | AAD56240. | 6e−53 | 135/430 | 202/430 | DhbF,Bacillus subtilis |
| | | | | 1,2378aa | | (31.4%) | (46.98%) | |
| | | | | BAB69380. | 7e−47 | 137/405 | 179/405 | non-ribosomal peptide |
| | | | | 1,1440aa | | (33.83%) | (44.2%) | synthetase,Streptomyces avermitilis |
| 16 | NRPS- | 023C | 432aa | AAD56240. | 6e−54 | 125/393 | 202/393 | DhbF,Bacillus subtilis |
| | Cdom | | | 1,2378aa | | (31.81%) | (51.4%) | |
| | | | | BAB69380. | 3e−46 | 124/400 | 180/400 | non-ribosomal peptide |
| | | | | 1,1440aa | | (31%) | (45%) | synthetase,Streptomyces avermitilis |
| | | | | AAG31130. | 1e−44 | 128/436 | 196/436 | MxcG,Stigmatella aurantiaca |
| | | | | 1,1456aa | | (29.36%) | (44.95%) | |
| 18 | NRPS- | A410 | 431aa | AAD56240. | 1e−68 | 149/428 | 213/428 | DhbF,Bacillus subtilis |
| | Cdom | | | 1,2378aa | | (34.81%) | (49.77%) | |
| | | | | AAG31130. | 5e−56 | 141/424 | 194/424 | MxcG,Stigmatella aurantiaca |
| | | | | 1,1456aa | | (33.25%) | (45.75%) | |
| | | | | NP_627443. | 1e−55 | 144/446 | 199/446 | CDA peptide synthetase I,Streptomyces |
| | | | | 1,17463aa | | (32.29%) | (44.62%) | coelicolor |
| 20 | NRPS- | 070B | 435aa | AAD56240. | 7e−86 | 168/423 | 239/423 | DhbF,Bacillus subtilis |
| | Cdom | | | 1,2378aa | | (39.72%) | (56.5%) | |
| | | | | AAG31130. | 5e−83 | 174/415 | 237/415 | MxcG,Stigmatella aurantiaca |
| | | | | 1,1456aa | | (41.93%) | (57.11%) | |
| | | | | BAB69380. | 9e−71 | 155/406 | 215/406 | non-ribosomal peptide |
| | | | | 1,1440aa | | (38.18%) | (52.96%) | synthetase,Streptomyces avermitilis |
| 22 | NRPS- | CADA | 446aa | NP_627443. | 0.0 | 446/446 | 446/446 | CDA peptide synthetase I,Streptomyces |
| | Cdom | | | 1,7463aa | | (100%) | (100%) | coelicolor |
| | | | | AAD56240. | 2e−70 | 159/450 | 236/450 | DhbF,Bacillus subtilis |
| | | | | 1,2378aa | | (35.33%) | (52.44%) | |
| | | | | T14591, | 6e−67 | 171/448 | 224/448 | actinomycin synthetase,Streptomyces |
| | | | | 2611aa | | (38.17%) | (50%) | chrysomallus |

As used herein, the term “adenylating enzyme” or ADLE, means member of a family of proteins involved in N-acyl capping for lipopeptide biosynthesis. Representative adenylating enzymes of the invention include the adenylating enzyme residing in the ramoplanin biosynthetic locus from Actinoplanes sp. ATCC 33076 (SEQ ID NO: 22), the adenylating enzyme residing in the A21978C locus in Streptomyces roseosporus NRRL 11379 (SEQ ID NO: 24), the adenylating enzyme residing in a lipopeptide biosynthetic locus in Streptomyces ghanaensis NRRL B-12104 (SEQ ID NO: 26), the adenylating enzyme residing in a lipopeptide biosynthetic locus in Streptomyces refuineus NRRL 3143 (SEQ ID NO: 28), the adenylating enzyme residing in a lipopeptide biosynthetic locus in Streptomyces aizunensis NRRL B-11277 (SEQ ID NO: 30), and the adenylating enzyme residing in a lipopeptide biosynthetic locus in Actinoplanes nipponensis FD 24834 ATCC 31145 (SEQ ID NO: 32). The adenylating enzyme may be a portion of a fusion protein, for example, the adenylating enzyme residing in the A54145 locus in Streptomyces fradiae ATCC 18158 is residues 1 to 648 of a fusion protein designated ADLF (SEQ ID NO: 34).

The adenylating enzyme is defined structurally as a polypeptide sequence that produces an alignment with at least 55% identity to the following consensus sequence using the BLASTP 2.0.10 algorithm (with the filter option -F set to false, the gap opening penalty -G set to 11, the gap extension penalty -E set to 1, and all remaining options set to default values):

>Consensus sequence 3
vsavmvdIaagpsvpaaLRahAearPdRtAvvfVrDtdradgtasLsYae
LDrrARavAvwLrarlapGdRvLLLhPaGpeFvaAyLgCLYAGIvAVPAP
LPGgysherrRVvgIAaDagagaVLTdadteAeVreWIaEtGLpgLPVIA
vDpIAadgDPgaWrpPgIradtVAvLQYTSGSTGsPKGVvVTHgNLLaNa
rsLsrsfgItedtvfGGWLPIyHDMGLfGILIPaLfIGatvVLMSPsAFI
rRPhIWLrIIDRfgvvfSAAPDFAYDLCvRRVtDEQiAgLDLSRWRwAaN
GSEPIrAaTIRaFaeRFApAGLRpeaLtPCYGLAEATIfVSgkSagPIrt
rrVDpaaLEdHrfeeAvpGrpaREiVsCGrvpdIevRIVDPgtgrpLPdG
aVGEIwLRGpSVaaGYWgrpEataetFgavtDGgDGPwLRTGDLGALYeG
ELYVTGRiKEILiVhGRNIYPhDiEhELRAaHdELagavGAaFaVpapGg
GeEvIVVvHEVrprvpaDeIpaLAsAmRaTvaREFGvpaagVvLvRRGTV
rRTTSGKvQRramReLFItGeLapvHaelgphlqaaaagearaatSIAPa
Stv

where consensus sequence 3 is based on the ADLE polypeptide sequences of the DAPT, A410, 009H, 024A, RAMO and 023C lipopeptide loci as described herein above and residues 1 to 648 of the ADLF (as defined hereinafter) polypeptide sequence of the A541 lipopeptide locus. Consensus sequence 3 was generated as described above in relation to consensus sequences 1 and 2.

A “polynucleotide encoding an adenylating enzyme” or a “polynucleotide encoding ADLE” refers to a polynucleotide encoding a member of the ADLE family of proteins involved in N-acyl capping for lipopeptide biosynthesis. Representative polynucleotides encoding adenylating enzymes of the invention include the polynucleotide encoding the adenylating enzyme residing in the ramoplanin biosynthetic locus from Actinoplanes sp. ATCC 33076 (SEQ ID NO: 21), the polynucleotide encoding the adenylating enzyme residing in the A21978C locus in Streptomyces roseosporus NRRL 11379 (SEQ ID NO: 23), the polynucleotide encoding the adenylating enzyme residing in a lipopeptide biosynthetic locus in Streptomyces ghanaensis NRRL B-12104 (SEQ ID NO: 25), the polynucleotide encoding the adenylating enzyme residing in a lipopeptide biosynthetic locus in Streptomyces refuineus NRRL 3143 (SEQ ID NO: 27), the polynucleotide encoding the adenylating enzyme residing in a lipopeptide biosynthetic locus in Streptomyces aizunensis NRRL B-11277 (SEQ ID NO: 29), and the polynucleotide encoding the adenylating enzyme residing in a lipopeptide biosynthetic locus in Actinoplanes nipponensis FD 24834 ATCC 31145 (SEQ ID NO: 31). The nucleotide encoding an adenylating enzyme may be a portion of a gene encoding a fusion protein, for example, the nucleotide encoding the adenylating enzyme residing in the A54145 locus in Streptomyces fradiae ATCC 18158 is residues 1 to 1944 of the nucleotide encoding a fusion protein designated ADLF (SEQ ID NO: 33). The ADLE portion of the ADLF fusion protein is sometimes designated with an asterisk “★” in the figures.

The ADLE polypeptides of SEQ ID NOS: 24, 26, 28, 30, 31, 32, and residues 1 to 648 of SEQ ID NO 34, i.e. the portion of the ADLF fusion protein representing an ADLE protein, were compared using the BLASTP algorithm with the default parameters to the sequences of the National Center for Biotechnology Information (NCBI) nonredundant protein database and to sequences of the DECIPHER® database of microbial genes, pathways and natural products (Ecopia BioSciences Inc., St-Laurent, Canada). The accession numbers of the top GenBank hits of this BLAST analysis are presented in Table 2 along with the corresponding E values.

TABLE 2

| | | | | GenBank | prob- | % | % | proposed function of GenBank |
|---|---|---|---|---|---|---|---|---|
| SEQ id | Family | Locus | #aa | homology | ability | identity | similarity | match |
| 24 | ADLE | RAMO | 584aa | AAG02359. | 1e−95 | 235/572 | 291/572 | peptide synthetase, Streptomyces |
| | | | | 1,2675aa | | (41.08%) | (50.87%) | verticillus |
| | | | | BAB69270. | 2e−92 | 224/563 | 279/563 | non-ribosomal peptide synthetase/ |
| | | | | 1,1261aa | | (39.79%) | (49.56%) | acyl-CoA dehydrogenase |
| | | | | | | | | fusion, Streptomyces avermiltilis |
| | | | | T18551, | 2e−79 | 199/576 | 273/576 | saframycin Mx1 synthetase |
| | | | | 1770aa | | (34.55%) | (47.4%) | B, Myxococcus xanthus |
| 26 | ADLE | DAPT | 600aa | BAB69270. | 1e−92 | 206/477 | 263/477 | non-ribosomal peptide synthetase/ |
| | | | | 1,1261aa | | (43.19%) (55.14%) | acyl-CoA dehydrogenase | |
| | | | | | | | | fusion, Streptomyces avermitilis |
| | | | | T18551, | 2e−87 | 201/540 | 273/540 | saframycin Mx1 synthetase |
| | | | | 1770aa | | (37.22%) | (50.56%) | B, Myxococcus xanthus |
| | | | | AAG02359. | 5e−81 | 194/534 | 263/534 | peptide synthetase, Streptomyces |
| | | | | 1,2675aa | | (36.33%) | (49.25%) | verticillus |
| 28 | ADLE | 009H | 594aa | BAB69270. | 1e−115 | 244/577 | 316/577 | non-ribosomal peptide synthetase/ |
| | | | | 1,1261aa | | (42.29%) | (54.77%) | acyl-CoA dehydrogenase |
| | | | | | | | | fusion, Streptomyces averniltilis |
| | | | | NP_630013. | 3e−92 | 225/575 | 296/575 | polyketide synthase, Streptomyces |
| | | | | 1,2297aa | | (39.13%) | (51.48%) | coelicolor |
| | | | | T18551. | 3e−91 | 210/565 | 296/565 | saframycin Mx1 synthetase |
| | | | | 1770aa | | (37.17%) | (52.39%) | B, Myxococcus xanthus |
| 30 | ADLE | 024A | 601aa | BAB69270. | 1e−112 | 257/594 | 322/594 | non-ribosomal peptide synthetase/ |
| | | | | 1,1261aa | | (43.27%) | (54.21%) | acyl-CoA dehydrogenase |
| | | | | | | | | fusion, Streptomyces avermitilis |
| | | | | NP_630013. | 1e−103 | 261/640 | 338/640 | polyketide synthase, Streptomyces |
| | | | | 1,2297aa | | (40.78%) | (52.81%) | coelicolor |
| | | | | AAN85512. | 1e−102 | 242/578 | 307/578 | non ribosomal peptide |
| | | | | 1,1745aa | | (41.87%) | (53.11%) | synthetase, Streptomyces |
| | | | | | | | | atroolivaceus |
| 32 | ADLE | 023C | 580aa | BAB69270. | 1e−136 | 266/572 | 333/572 | non-ribosomal peptide synthetase/ |
| | | | | 1,1261aa | | (46.5%) | (58.22%) | acyl-CoA dehydrogenase |
| | | | | | | | | fusion, Streptomyces avermitilis |
| | | | | ZP_00110248. | 1e−112 | 226/560 | 318/560 | hypothetical protein, Nostoc |
| | | | | 1,1204aa | | (40.36%) | (56.79%) | punctiforme |
| | | | | BAB69218. | 1e−109 | 230/572 | 314/572 | non-ribosomal peptide |
| | | | | 1,574aa | | (40.21%) | (54.9%) | synthetase, Streptomyces |
| | | | | | | | | avermitilis |
| 34 | ADLE | A410 | 588aa | BAB69270. | 1e−117 | 251/596 | 321/596 | non-ribosomal peptide synthetase/ |
| | | | | 1,1261aa | | (42.11%) | (53.86%) | acyl-CoA dehydrogenase |
| | | | | | | | | fusion, Streptomyces avermitilis |
| | | | | AAG02359. | 1e−103 | 233/588 | 315/588 | peptide synthetase, Streptomyces |
| | | | | 1,2675aa | | (39.63%) | (53.57%) | verticillus |
| | | | | ZP_00124327. | 1e−102 | 238/570 | 318/570 | hypothetical protein, Pseudomonas |
| | | | | 1,4339aa | | (41.75%) (55.79%) | syringae | |
| 36 | ADLF | A541 | 723aa | BAB69270. | 1e−108 | 261/629 | 320/629 | non-ribosomal peptide synthetase/ |
| | | | | 1,1261aa | | (41.49%) | (50.87%) | acyl-CoA dehydrogenase |
| | | | | | | | | fusion, Streptomyces avermiltilis |
| | | | | NP_251114. | 1e−101 | 251/679 | 346/679 | non-ribosomal peptide |
| | | | | 1,4342aa | | (36.97%) | (50.96%) | synthetase, Pseudomonas aeruginosa |
| | | | | AAN85512. | 1e−101 | 261/684 | 344/684 | non ribosomal peptide |
| | | | | 1,1745aa | | (38.16%) | (50.29%) | synthetase, Streptomyces |
| | | | | | | | | atroolivaceus |
| 36* | ADLE | A541 | 608aa | BAB69270. | 1e−108 | 254/594 | 311/594 | non-ribosomal peptide synthetase/ |
| | domain | | | 1,1261aa | | (42.76%) | (52.36%) | acyl-CoA dehydrogenase |
| | | | | | | | | fusion, Streptomyces avermitilis |
| | of | | | AAN85512. | 9e−99 | 238/581 | 309/581 | non-ribosmal peptide |
| | A541_ADL | 1,1745aa | | (40.96%) | (53.18%) | synthetase, Streptomyces | | |
| | F | | | | | | | atroolivaceus |
| | | | | NP_251114. | 6e−98 | 231/591 | 313/591 | non-ribosomal peptide |
| | | | | 1,4342aa | | (39.09%) | (52.96%) | synthetase, Pseudomonas aeruginosa |

As used herein, the term acyl carrier protein or ACPH refers to a member of a family of proteins involved in N-acyl capping for lipopeptide biosynthesis. Representative acyl carrier proteins of the invention include the acyl carrier protein residing in the ramoplanin biosynthetic locus from Actinoplanes sp. ATCC 33076 (SEQ ID NO: 36), the acyl carrier protein residing in the A21978C locus in Streptomyces roseosporus NRRL 11379 (SEQ ID NO: 38), the acyl carrier protein residing in a lipopeptide biosynthetic locus in Streptomyces ghanaensis NRRL B-12104 (SEQ ID NO: 40), the acyl carrier protein residing in a lipopeptide biosynthetic locus in Streptomyces refuineus NRRL 3143 (SEQ ID NO: 42), the acyl carrier protein residing in a lipopeptide biosynthetic locus in Streptomyces aizunensis NRRL B-1 1277 (SEQ ID NO: 44), and the acyl carrier protein residing in a lipopeptide biosynthetic locus in Actinoplanes nipponensis FD 24834 ATCC 31145 (SEQ ID NO: 46). The acyl carrier protein may be a portion of a fusion protein, for example, the acyl carrier protein residing in the A54145 locus in Streptomyces fradiae ATCC 18158 is residues 649 to 743 of a fusion protein designated ADLF (SEQ ID NO: 34). The ACPH portion of the ADLF fusion protein is sometimes designated with a double asterisk “★★” in the figures.

The acyl carrier protein (ACPH) of the invention is defined structurally as a polypeptide sequence that produces an alignment with at least 50% identity to the following consensus sequence using the BLASTP 2.0.10 algorithm (with the filter option -F set to false, the gap opening penalty -G set to 11, the gap extension penalty -E set to 1, and all remaining options set to default values):

>Consensus sequence 4
MsdItappArhTPeelRaWLrecvAdyVgIppaeIatDvPLtdYGLDSVy
alaLCAeiEDhIGievdptLLWDhPTIdeLsaaLaPrlarr

where consensus sequence 4 is based on the ACPH polypeptide sequences of the DAPT, A410, 009H, 024A, RAMO, 023C lipopeptide loci and residues 649 to 743 of the ADLF polypeptide sequence of the A541 lipopeptide locus. A “polynucleotide encoding an ACPH” is defined as a nucleotide sequence encoding an acyl carrier protein as defined above. Consensus sequence 4 was generated as described above in relation to consensus sequences 1 and 2.

A “polynucleotide encoding an acyl carrier protein” or a “polynucleotide encoding ACPH” refers to a polynucleotide encoding a member of the ACPH family of proteins involved in N-acyl capping for lipopeptide biosynthesis. Representative polynucleotides encoding acyl carrier proteins of the invention include the polynucleotide encoding the acyl carrier protein residing in the ramoplanin biosynthetic locus from Actinoplanes sp. ATCC 33076 (SEQ ID NO: 35), the polynucleotide encoding an acyl carrier protein residing in the A21978C locus in Streptomyces roseosporus NRRL 11379 (SEQ ID NO: 37), the polynucleotide encoding the acyl carrier protein residing in a lipopeptide biosynthetic locus in Streptomyces ghanaensis NRRL B-12104 (SEQ ID NO: 39), the polynucleotide encoding an acyl carrier protein residing in a lipopeptide biosynthetic locus in Streptomyces refuineus NRRL 3143 (SEQ ID NO: 41), the polynucleotide encoding an acyl carrier protein residing in a lipopeptide biosynthetic locus in Streptomyces aizunensis NRRL B-1 1277 (SEQ ID NO: 43), and the polynucleotide encoding the acyl carrier protein residing in a lipopeptide biosynthetic locus in Actinoplanes nipponensis FD 24834 ATCC 31145 (SEQ ID NO: 45). The polynucleotide encoding an acyl carrier protein may be a portion of a gene encoding a fusion protein, for example, the polynucleotide encoding the acyl carrier protein residing in the A54145 locus in Streptomyces fradiae ATCC 18158 is residues 1945 to 2229 of the polynucleotide encoding fusion protein designated ADLF (SEQ ID NO: 33).

The ACPH polypeptides of SEQ ID NOS: 36, 38, 40, 42, 44 and 46, and residues 649 to 743 of SEQ ID NO: 34, i.e. the ACPH portion of the ADLF fusion protein, were compared using the BLASTP algorithm with the default parameters to the sequences of the National Center for Biotechnology Information (NCBI) nonredundant protein database and to sequences of the DECIPHER® database of microbial genes, pathways and natural products (Ecopia BioSciences Inc., St-Laurent, Canada). The accession numbers of the top GenBank hits of this BLAST analysis are presented in Table 3 along with the corresponding E values.

TABLE 3

| | | | | | | | | |
|---|---|---|---|---|---|---|---|---|
| | | | | GenBank | prob- | % | % | proposed function of GenBank |
| SEQ id | Family | Locus | #aa | homology | ability | identity | similarity | match |

| | | | | | | | | |
|---|---|---|---|---|---|---|---|---|
| 36** | ACPH | A541 | 61aa | AAF62883. | 5e−04 | 23/58 | 30/58 | epoD, Polyangium cellulosum |
| | domain | | | 1,7257aa | | (39.66%) | (51.72%) | |
| | of | | | AAF26920. | 7e−04 | 22/58 | 29/58 | polyketide synthase, Polyangium |
| | 541_ADLF | 1,1832aa | | (37.93%) | (50%) | cellulosum | | |
| | | | | NP_488099. | 0.002 | 18/51 | 28/51 | unknown protein, Nostoc sp. |
| | | | | 1,126aa | | (35.29%) | (54.9%) | |
| 38 | ACPH | RAMO | 90aa | BAB69272. | 1e−17 | 41/82 | 56/82 | hypothetical protein, |
| | | | | 1,88aa | | (50%) | (68.29%) | Streptomyces avermitilis |
| | | | | ZP_00107884. | 2e−09 | 28/76 | 49/76 | hypothetical protein, |
| | | | | 1,105aa | | (36.84%) | (64.47%) | Nostoc punctiforme |
| | | | | NP_488099. | 2e−08 | 27/75 | 44/75 | unknown protein, Nostoc sp. |
| | | | | 1,126aa | | (36%) | (58.67%) | |
| 40 | ACPH | DAPT | 89aa | BAB69272. | 8e−10 | 28/81 | 47.81 | hypothetical protein, |
| | | | | 1,88aa | | (34.57%) | (58.02%) | Streptomyces avermitilis |
| | | | | NP_488099. | 6e−07 | 26/82 | 41/82 | unknown protein, Nostoc sp. |
| | | | | 1,126aa | | (31.71%) | (50%) | |
| | | | | ZP_00003083. | 9e−06 | 26/84 | 48/84 | hypothetical protein, |
| | | | | 1,2544aa | | (30.95%) | (57.14%) | Nitrosomonas europaea |
| 42 | ACPH | 009H | 90aa | BAB69272. | 1e−14 | 39/86 | 53/86 | hypothetical protein, |
| | | | | 1,88aa | | (45.35%) | (61.63%) | Streptomyces avermitilis |
| | | | | ZP_00107884. | 1e−09 | 31/74 | 45/74 | hypothetical protein, |
| | | | | 1,105aa | | (41.89%) | (60.81%) | Nostoc punctiforme |
| | | | | AAA03658. | 4e−09 | 31/78 | 44/78 | polyketide synthase, |
| | | | | 1,506aa | | (39.74%) | (56.41%) | Anabaena sp. |
| 44 | ACPH | 024A | 99aa | BAB69272. | 4e−09 | 29/77 | 43/77 | hypothetical protein, |
| | | | | 1,88aa | | (37.66%) | (55.84%) | Streptomyces avermitilis |
| | | | | NP_488099. | 2e−05 | 24/73 | 37/73 | unknown protein, Nostoc sp. |
| | | | | 1,126aa | | (32.88%) | (50.68%) | |
| | | | | AAF62883. | 2e−04 | 24/71 | 37/71 | epoD, Polyangium cellulosum |
| | | | | 1,7257aa | | (33.8%) | (52.11%) | |
| 46 | ACPH | 023C | 91aa | BAB69272. | 3e−18 | 42/75 | 53/75 | hypothetical protein, |
| | | | | 1,88aa | | (56%) | (70.67%) | Streptomyces avermitilis |
| | | | | ZP_00107884. | 2e−09 | 27/71 | 44/71 | hypothetical protein, |
| | | | | 1,105aa | | (38.03%) | (61.97%) | Nostoc punctiforme |
| | | | | BAB69217. | 3e−07 | 26/69 | 43/69 | polyketide synthase, |
| | | | | 1,4809aa | | (37.68%) | (62.32%) | Streptomyces avermitilis |
| 48 | ACPH | A410 | 88aa | BAB69272. | 8e−18 | 41/81 | 57/81 | hypothetical protein, |
| | | | | 1,88aa | | (50.62%) | (70.37%) | Streptomyces avermitilis |
| | | | | NP_488099. | 6e−12 | 34/75 | 48/75 | unknown protein, Nostoc sp. |
| | | | | 1,126aa | (45.33%) | (64%) | | |
| | | | | BAB69217. | 3e−08 | 29/72 | 43/72 | polyketide synthase, |
| | | | | 1,4809aa | | (40.28%) | (59.72%) | Streptomyces avermitilis |

As used herein, the term “ADLF” refers to a single open reading frame located in the A54145 locus (SEQ ID NO: 33), where the single open reading frame is formed by the genes encoding the ADLE and ACPH proteins fused together. The gene product of the open reading frame of SEQ ID NO: 33 is provided in SEQ ID NO: 34 wherein residues 1 to 648 of SEQ ID NO: 34 represent an ADLE protein and residues 649 to 743 of SEQ ID NO: 34 represent an ACPH protein. It is expected that a similar fusion of ADLE and ACPH homologues may occur in other lipopeptide biosynthetic loci. It is also expected that other permutations of fusion proteins involving protein families of the invention may be found in lipopeptide loci, for example a fusion of ADLE and ACPH and the acyl-specific C-domain or a fusion of ACPH and the acyl specific C-domain.

Cosmid clones containing genes and proteins of the invention have been deposited with the International Depositary Authority of Canada, Bureau of Microbiology, Health Canada, 1015 Arlington Street, Winnipeg, Manitoba, Canada R3E 3R2 under the terms of the Budapest Treaty on the International Recognition of the Deposit of Microorganisms for Purposes of Patent Procedure. An E. coli DH10B strain harboring cosmid clone 008CH containing the ACPH gene and the acyl-specific C-domain in the biosynthetic locus for ramoplanin from Actinoplanes sp. ATCC 33076 was deposited on Sep. 19, 2001 and assigned accession number IDAC 190901-3. An E. coli DH10B strain harboring cosmid clone 008CO containing the ADLE gene in the biosynthetic locus for ramoplanin from Actinoplanes sp. ATCC 33076 was deposited on Sep. 19, 2001 and assigned accession number IDAC 190901-2. An E. coli DH10B strain harboring cosmid clone 024CK containing the ADLE and ACPH gene and the acyl-specific C-domain in the biosynthetic locus for the lipopeptide from Streptomyces refuineus subsp. thermotolerans was deposited on Feb. 26, 2002 and assigned accession number IDAC 260202-5. An E. coli DH10B strain harboring cosmid clone 184CM containing the ADLF fusion protein and the acyl-specific C-domain in the biosynthetic locus for A54145 lipopeptide from Streptomyces fradiae was deposited on Feb. 26, 2002 and assigned accession number IDAC 260202-1. The E. coli strain deposits are referred to herein as “the deposited strains”.

The sequences of the nucleotides encoding members of the protein families ADLE, ADLF, ACPH and the acyl specific C-domains of the invention present in the deposited strains as well as the amino acid sequences of the corresponding polypeptides are controlling in the event of any conflict with any description of sequences herein. A license may be required to make, use or sell the deposited strains, nucleic acids therein or compounds derived therefrom, and no such license is hereby granted.

As used herein, the term “a polypeptide involved in lipopeptide synthesis” refers to any polypeptide as defined herein as an acyl-specific C-domain, or an adenylating enzyme, or an acyl carrier protein. A “polynucleotide involved in lipopeptide synthesis” refers to a nucleotide sequence encoding a polypeptide involved in lipopeptide synthesis as defined herein.

The term “isolated” means that the material is removed from its original environment, e.g. the natural environment if it is naturally-occurring. For example, a naturally-occurring polynucleotide or polypeptide present in a living organism is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment.

As used herein, “a condition of high stringency” refers to any one of the hybridization conditions described herein, and include other “high stringency” conditions known in the art. In one condition, a polymer membrane containing immobilized denatured nucleic acids is first prehybridized for 30 minutes at 45° C. in a solution consisting of 0.9 M NaCl, 50 mM NaH₂PO₄, pH 7.0, 5.0 mM Na₂EDTA, 0.5% SDS, 10X Denhardt's, and 0.5 mg/ml polyriboadenylic acid. Approximately 2×10⁷ cpm (specific activity 4-9×10⁸ cpm/ug) of ³²P end-labeled oligonucleotide probe are then added to the solution. After 12-16 hours of incubation, the membrane is washed for 30 minutes at room temperature in 1×SET (150 mM NaCl, 20 mM Tris hydrochloride, pH 7.8, 1 mM Na₂EDTA) containing 0.5% SDS, followed by a 30 minute wash in fresh 1×SET at Tm-10° C. for the oligonucleotide probe, where Tm is the melting temperature of the probe. Stringency may be varied by conducting the hybridization at varying temperatures below the melting temperatures of the probes. The melting temperature of the probe may be calculated using the following formula: for oligonucleotide probes between 14 and 70 nucleotides in length, the melting temperature (Tm) in degrees Celcius may be calculated using the formula: Tm=81.5+16.6(log [Na+])+0.41(fraction G+C)−(600/N), where N is the length of the oligonucleotide. If the hybridization is carried out in a solution containing formamide, the melting temperature may be calculated using the equation Tm=81.5+16.6(log [Na+])+0.41 (fraction G+C)−(0.63% formamide)−(600/N), where N is the length of the probe. For probes over 200 nucleotides in length, the hybridization may be carried out at 15-25° C. below the Tm. For shorter probes, such as oligonucleotide probes, the hybridization may be conducted at 5-10° C. below the Tm. Preferably, the hybridization is conducted in 6×SSC for shorter probes and the hybridization is conducted in 50% formamide containing solutions for longer probes.

As used herein, the term “homology” refers to the optimal alignment of sequences (either nucleotides or amino acids), which may be conducted by computerized implementations of algorithms. “Homology”, with regard to polynucleotides, for example, may be determined by analysis with BLASTN version 2.0 using the default parameters. “Homology”, with respect to polypeptides (i.e., amino acids), may be determined using a program, such as BLASTP version 2.2.2 with the default parameters, which aligns the polypeptides or fragments being compared and determines the extent of amino acid identity or similarity between them. It will be appreciated that amino acid “homology” includes conservative substitutions, i.e. those that substitute a given amino acid in a polypeptide by another amino acid of similar characteristics. Typically seen as conservative substitutions are the following replacements: replacements of an aliphatic amino acid such as Ala, Val, Leu and lle with another aliphatic amino acid; replacement of a Ser with a Thr or vice versa; replacement of an acidic residue such as Asp or Glu with another acidic residue; replacement of a residue bearing an amide group, such as Asn or Gln, with another residue bearing an amide group; exchange of a basic residue such as Lys or Arg with another basic residue; and replacement of an aromatic residue such as Phe or Tyr with another aromatic residue. A “homology of 70% or higher” includes a homology of, for example, 70%, 75%, 80%, 85%, 90%, 95%, and up to 100% (identical) between two or more nucleotide or amino acid sequences. A “homology of at least 45%” includes a homology of, for example, 45%, 50%, 60%, 70%, 80%, 90%, and up to 100% (identical) between two or more nucleotide or amino acid sequences.

The present invention provides a method for detecting a polypeptide involved in lipopeptide biosynthesis or a polynucleotide encoding such a polypeptide.

In one embodiment, the method of the present invention provides one or more reference sequences and compares a candidate sequence (either a specific single candidate sequence or a candidate database sequence) with the one or more reference sequences. The sequence homology is determined for the sequences compared. A candidate sequence sharing at least 45% homology to one or more reference sequences is considered to be a candidate polypeptide or a candidate polynucleotide encoding a candidate polypeptide which is involved in lipopeptide biosynthesis. Preferably, a candidate polypeptide sequence sharing 45% homology to consensus sequences 1 or 2, is considered as a candidate acyl-specific C-domain polypeptide, a candidate polypeptide sequence sharing 55% homology to consensus sequence 3 is considered a candidate adenylating enzyme, a candidate polypeptide sequence 50% homology to consensus 4 is considered a candidate acyl-carrier protein. The involvement of these identified sequences in lipopeptide biosynthesis may be confirmed by first expressing the polypeptide from the polynucleotide candidates and performing the function analysis according to methods known in the art and as described herein in Examples 1-2.

In another embodiment of the invention, the subject method compares one or more reference sequences against sequences within a candidate database of a specific organism. This will determine whether the specific organism may contain a polypeptide involved in lipopeptide biosynthesis or a polynucleotide encoding such a polypeptide. If it is determined that a specific organism may contain such a polynucleotide sequence encoding a polypeptide for lipopeptide biosynthesis, proteins from the candidate database (e.g., a part of the whole genome sequence) may be expressed and analyzed according to methods known in the art and as described herein in Examples 1-2.

a preferred embodiment, the reference sequences used in the subject method are selected from the group consisting of polynucleotide or polypeptide sequences representing: an acyl-specific C-domain, an ADLE, an ACPH, and an ADLF in one or more of the biosynthetic loci selected from the group consisting of RAMO, DAPT, A541, 009H, 024A, 023C, A410, 070B and CADA.

In another preferred embodiment, the reference sequences may further include one or more reference polypeptides having at least 45% sequence homology to SEQ ID NO: 1 or SEQ ID NO: 2, one or more reference polypeptides having at least 55% sequence homology to SEQ ID NO: 3, one or more reference polypeptides having at least 50% sequence homology to SEQ ID NO: 4, or one or more reference polynucleotides encoding such polypeptide sequences.

Also within the scope of the present invention are a memory system for storing data that can be accessed by a computer, a computer readable medium comprising a computer program and data for sequence comparison, and a computer system for performing sequence comparison of the present invention.

The computer system of the present invention will provide one or more reference polynucleotide or polypeptide sequences selected from the group consisting of polynucleotide or polypeptide sequences representing an acyl-specific C-domain, an adenylating enzyme (ADLE) or an acyl carrier protein ACPH or a fusion of the two (ADLF) in one or more of the biosynthetic loci selected from RAMO, DAPT, A541, 009H, 024A, 023C, A410, 070B and CADA.

Additionally or alternatively, the computer system of the present invention will provide one or more reference polypeptides comprising the consensus sequences of the present invention, i.e. one or more reference polypeptides having at least 45% sequence homology to SEQ ID NO: 1 or SEQ ID NO: 2, one or more reference polypeptides having at least 55% sequence homology to SEQ ID NO: 3, one or more reference polypeptides having at least 50% sequence homology to SEQ ID NO: 4, or one or more reference polynucleotides encoding such polypeptide sequences.

The computer system of the invention may also provide candidate polynucleotide or polypeptide sequence(s). The candidate polynucleotide or polypeptide may exist as a specific single sequence or it may be a candidate database, e.g., a part of the entire genome sequence of an organism, or protein family sequences.

The computer system of the invention will perform sequence comparison between one or more candidate sequences and one or more reference sequences. The computer system will also determine the level of homology of two or more sequences compared and identify a candidate sequence which shares at least 45% homology with a SEQ ID NO: 1 or SEQ ID NO: 2, and in some embodiments additionally identify a candidate sequence which shares at least 55% homology with SEQ ID NO: 3 or a candidate sequence which shares at least 50% homology with SEQ ID NO: 4.

The memory and computer system of the present invention permits the quick development of methods to search candidate databases and individual candidate sequences for their sequence homology against one or more reference sequences. In addition, the memory and computer system of the present invention will also permit the prediction of protein sequences from polynucleotide sequences, the prediction of homologous protein domains between two or more polypeptides, and the analysis of structure and function from sequence data.

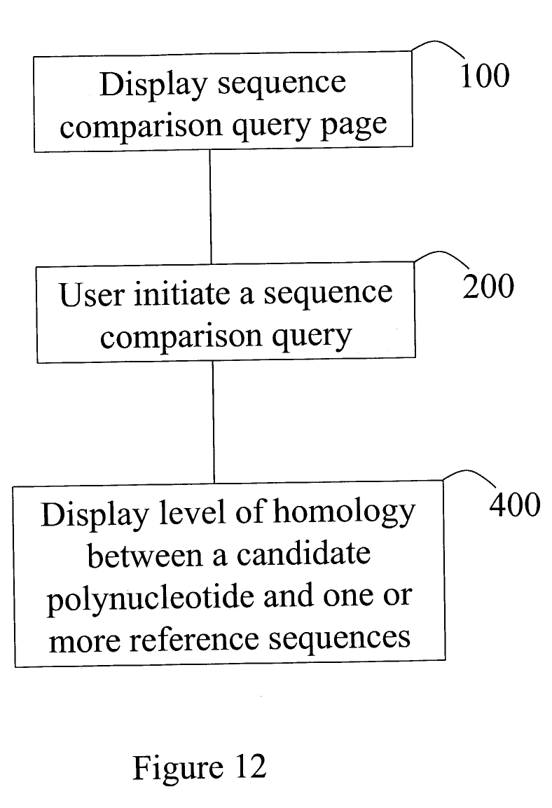

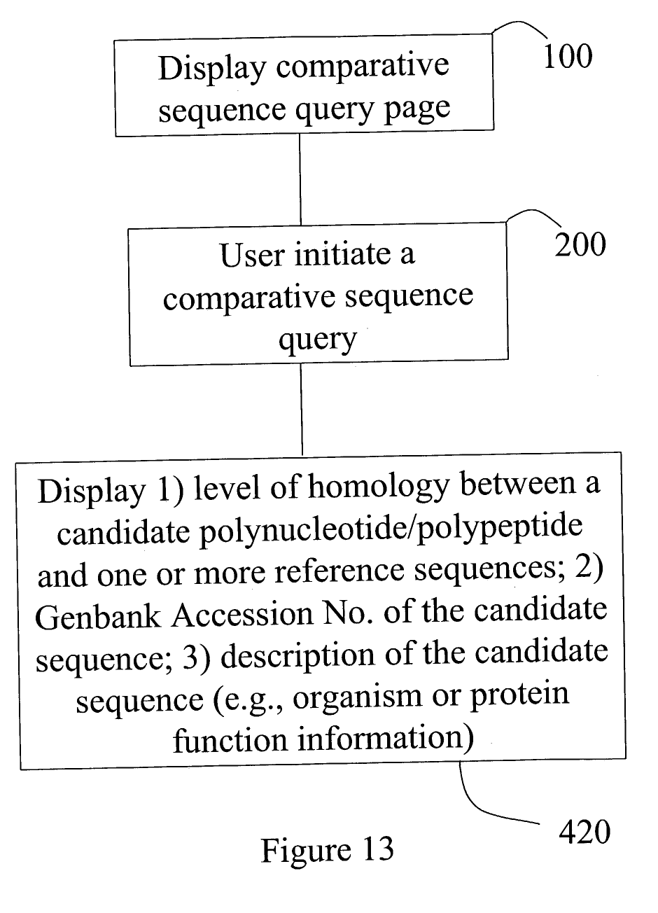

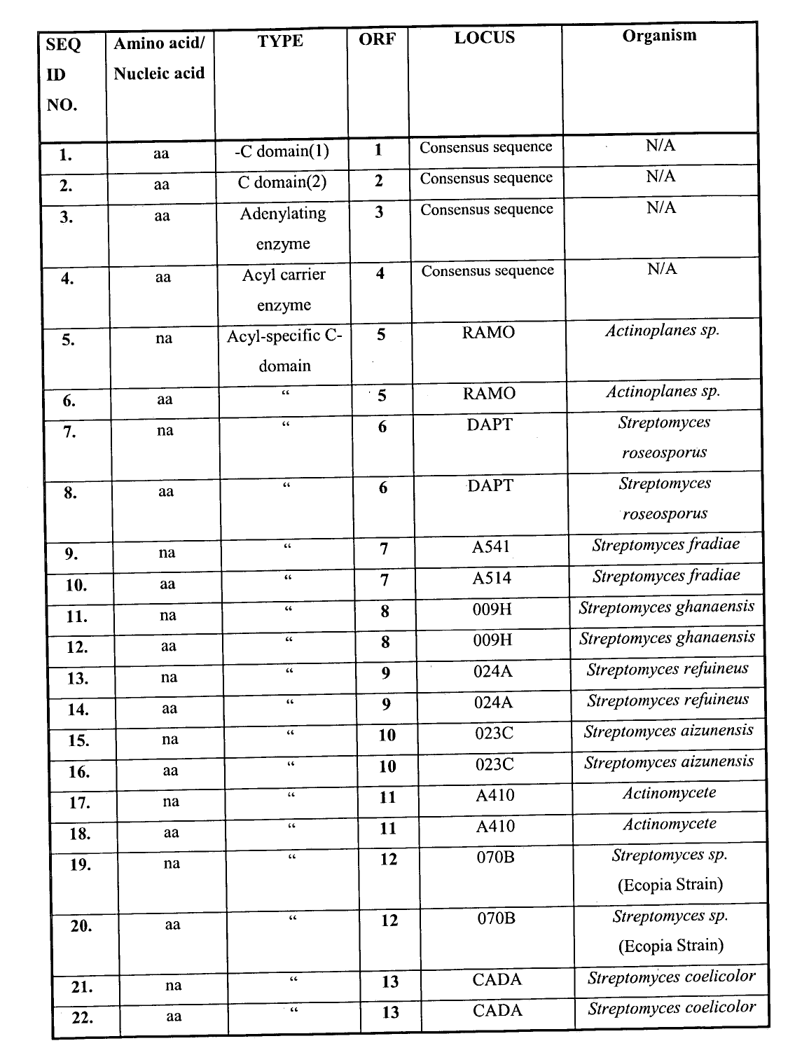

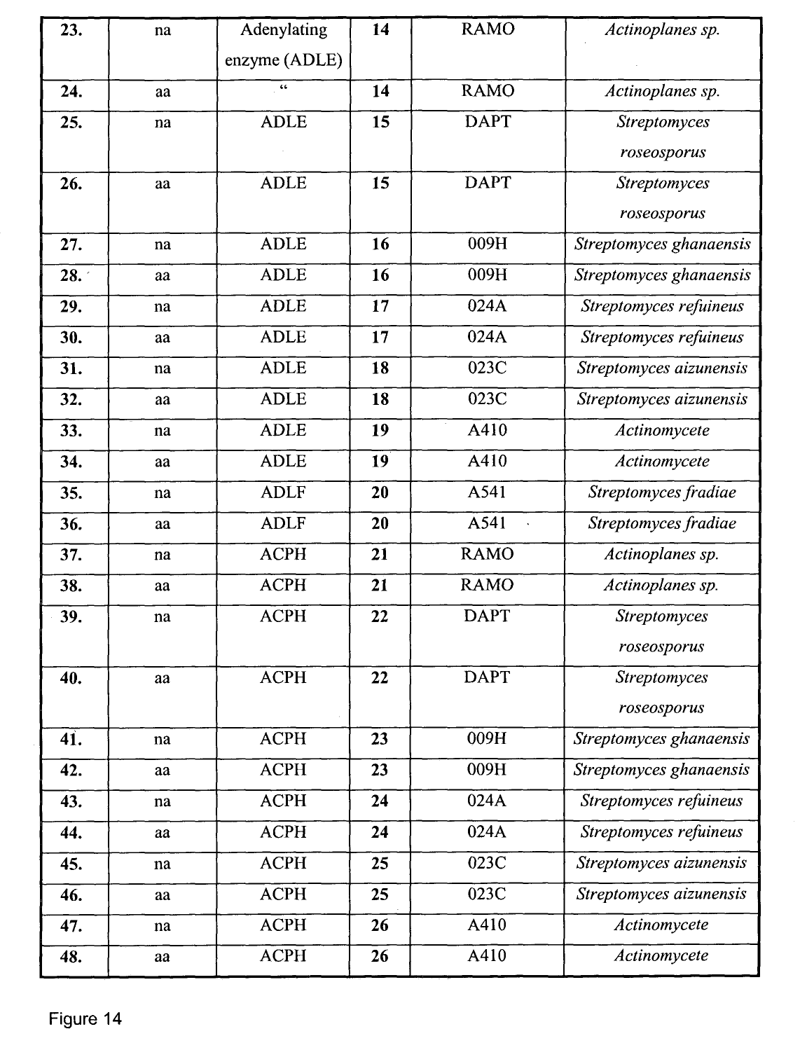

The computer may be programmed to implement a process for effecting the identification, analyses, or modeling of a sequence of a polypeptide or a polynucleotide. In one embodiment the memory of the present invention contains data representing a polypeptide with 70% sequence homology to any one sequence selected from the group consisting of: SEQ ID NOs. 1, 2, 3, 4, 6, 8, 10, 12, 14, 16, 18, 20, 22, 24, 26, 28, 30, 32, 34, 36, 38, 40, 42, 44, 46, and 48. The preferred process by which data for a source database according to the present invention may be obtained is illustrated in FIGS. 12 and 13.

One use of the memory and computer system involves studying an organism's genome (e.g., database candidate sequences) to determine the sequence homology between the polynucleotide/polypeptide sequences in the genome and one or more reference polynucleotide or polypeptide sequences. Such information is of significant interest in assessing whether an organism contains a lipopeptide biosynthesis locus or any polynucleotide/polypeptide involved in lipopeptide biosynthesis.

Another use of the memory and computer system involves studying one or more specific candidate sequences to determine the sequence homology between the specific candidate polynucleotide/polypeptide sequences and one or more reference polynucleotide or polypeptide sequences. Such information helps to determine whether the specific candidate sequence is involved in lipopeptide biosynthesis.

Where a specific polynucleotide candidate sequence or polynucleotide database candidate sequences are being analyzed, the memory and computer system may permit the prediction of an Open Reading Frame (ORF) from a candidate sequence. The ORF corresponds to a nucleotide sequence which could potentially be translated into a polypeptide. Such a stretch of sequence is uninterrupted by a stop codon. An ORF that represents the coding sequence for a full protein generally begins with an ATG “start” codon and terminates with one of the three “stop” codons. For the purposes of this application, an ORF may be any part of a coding sequence, with or without start and/or stop codons. For an ORF to be considered as a good candidate for coding for a bona fide cellular protein, a minimum size requirement is often set, for example, a stretch of DNA that would code for a protein of 50 amino acids or more.

To make the above sequence information manipulation easy to perform and understand, sophisticated computer database systems may be used. In one embodiment, the reference sequences are electronically recorded and annotated with information available from public sequence databases. Examples of such databases include GenBank (NCBI) and the Comprehensive Microbial Resource database (The Institute for Genomic Research). The resulting information is stored in a relational database that may be employed to determine homologies between the reference sequences and genes within and among genomes.

To identify homologies between the sequences, one or more sequence alignment algorithms such as BLAST (Basic Local Alignment Search Tool) or FAST (using the Smith-Waterman algorithm) may be employed. In a particularly preferred embodiment, these two alignment protocols are used in combination. Both of these algorithms look for regions of similarity between two sequences; the Smith-Waterman algorithm is generally more tolerant of gaps, and is used to provide a higher resolution match after the BLAST search provides a preliminary match. These algorithms determine (1) alignment between similar regions of the two sequences, and (2) a percent identity between sequences. For example, alignment may be calculated by matching, base-by-base or amino acid-by-amino acid, the regions of substantial similarity.

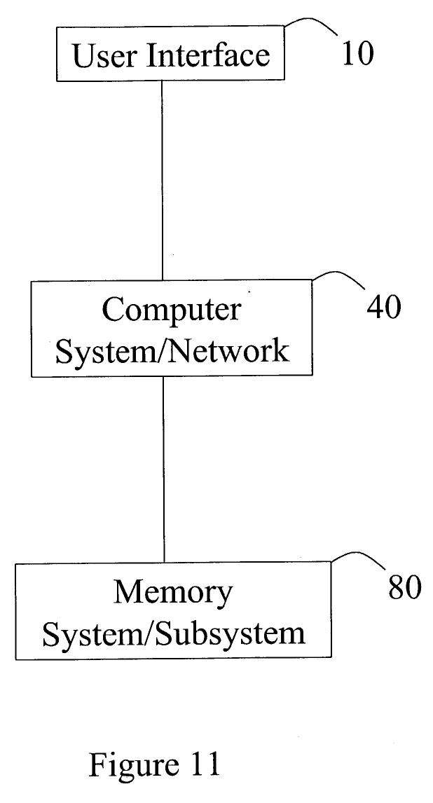

FIG. 11 is a block diagram of a computer system according to one embodiment of the invention. The system shown in FIG. 11 for performing the sequence comparison processing of the invention may be a general purpose computer used alone or in connection with a specialized processing computer. Such processing may be performed by a single platform or by a distributed processing platform. In addition, such processing and functionality can be implemented in the form of special purpose hardware or in the form of software being run by a general purpose computer. Any data handled in such processing or created as a result of such processing can be stored in a temporary memory, such as in the RAM of a given computer system or subsystem. In addition, or in the alternative, such data may be stored in longer-term storage devices, for example, magnetic disks, rewritable optical disks and so on. For purposes of the disclosure herein, computer-readable media may comprise any form of data storage mechanism, including such existing memory technologies as well as hardware or circuit representations of such structures and of such data.

The computer system 40 (FIG. 11) may include an operating system (e.g., UNIX) on which runs a relational database management system, a World Wide Web application, and a World Wide Web server. The software on the computer system may assume numerous configurations. For example, it may be provided on a single machine or distributed over multiple machines.

World Wide Web application includes the executable code necessary for generation of database language statements [e.g., Standard Query Language (SQL) statements]. Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers.

A World Wide Web browser may be used for providing a user interface 10 (FIG. 11). Through the Web browser, a user may construct search requests for retrieving data from a sequence database and/or a genomic database. Thus, the user will typically point and click to user interface elements such as buttons, pull down menus, scroll bars, etc. conventionally employed in graphical user interfaces. The requests so formulated with the user's Web browser are transmitted to a Web application which formats them to produce a query that can be employed to extract the pertinent information from sequence databases or genomic databases.

When network 40 employs a World Wide Web server, it supports a TCP/IP protocol. Local networks such as this are sometimes referred to as “Intranets.” An advantage of such Intranets is that they allow easy communication with public domain databases residing on the World Wide Web (e.g., the GenBank World Wide Web site). Thus, in a particular preferred embodiment of the present invention, users can directly access data (via Hypertext links for example) residing on Internet databases using a HTML interface provided by Web browsers and Web servers.

### EXAMPLE 1

Conserved Genes and Proteins Involved in N-acylation in Lipopeptides

The acyl-specific C-domains and ADLE, ADLF and ACPH protein families of the invention were discovered by identifying, characterizing and comparing several full-length biosynthetic loci, each producing a lipopeptide of known structure and each residing in a microorganism reported to produce the lipopeptide of known structure.

RAMO: Ramoplanin is a lipopeptide produced by Actinoplanes sp. ATCC 33076 (see US Pat. No. 4,303,646). Ramoplanin is a glycosylated lipodepsipeptide of known structure (see, for example, U.S. Pat. No. 4,427,656). The full-length biosynthetic locus for ramoplanin from Actinoplanes sp. (RAMO) was cloned and sequenced (FIG.. 1a). The open reading frames in RAMO were identified and a function was attributed to each protein encoded by the open reading frames. RAMO is described in detail in co-pending U.S. application Ser. No. 09/976,059 USSN and in PCT international application PCT/CA01/01462, published as WO 02/31155.

DAPT: A21978C is a lipopeptide produced by Streptomyces roseosporus. The structure of A21978C is known. While some progress has been reported towards elucidation of the biosynthetic locus responsible for the production of A21978C in Streptomyces roseosporus (DAPT), the full locus was not known. Transposon mutagenesis techniques had been performed to locate DAPT [McHenney et al. (1998) J. Bact. Vol. 180 pp. 143-151] and DNA fragments derived therefrom had been used for insertional mutagenesis experiments that demonstrated inactivation of A21978C production. Analysis of the DNA sequence of the fragments revealed the presence of NRPS genes involved in the biosynthesis A21978C. This genetic and biological data demonstrated beyond doubt that the identified pathway was indeed responsible for A21978C expression. However, the full biosynthetic locus for A21978C had not been reported.

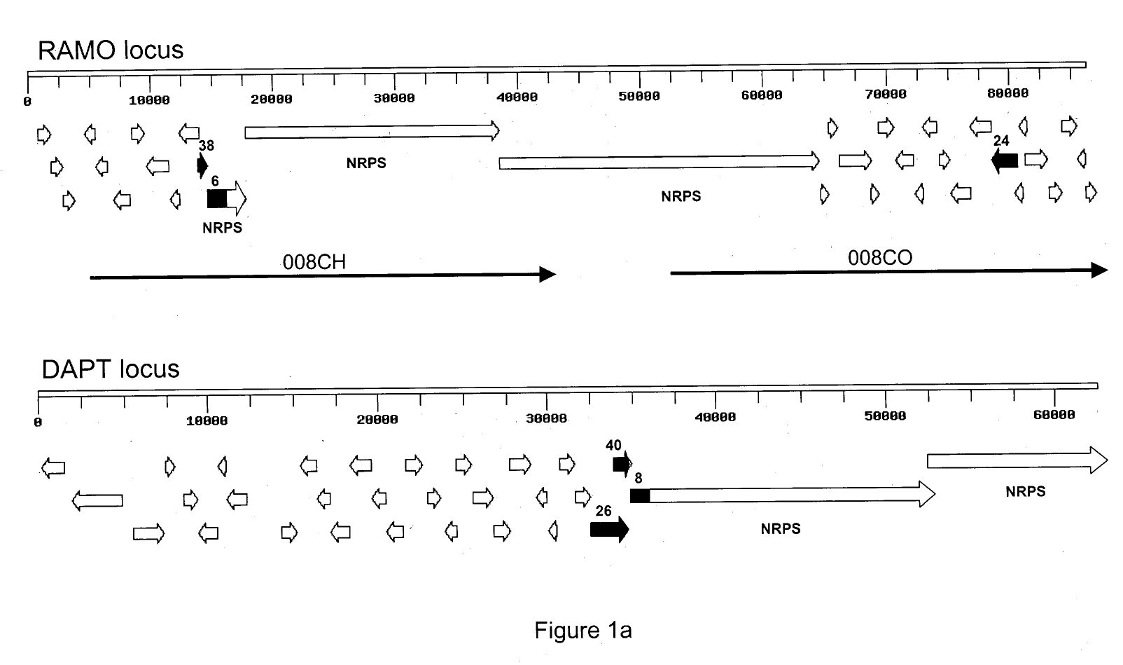

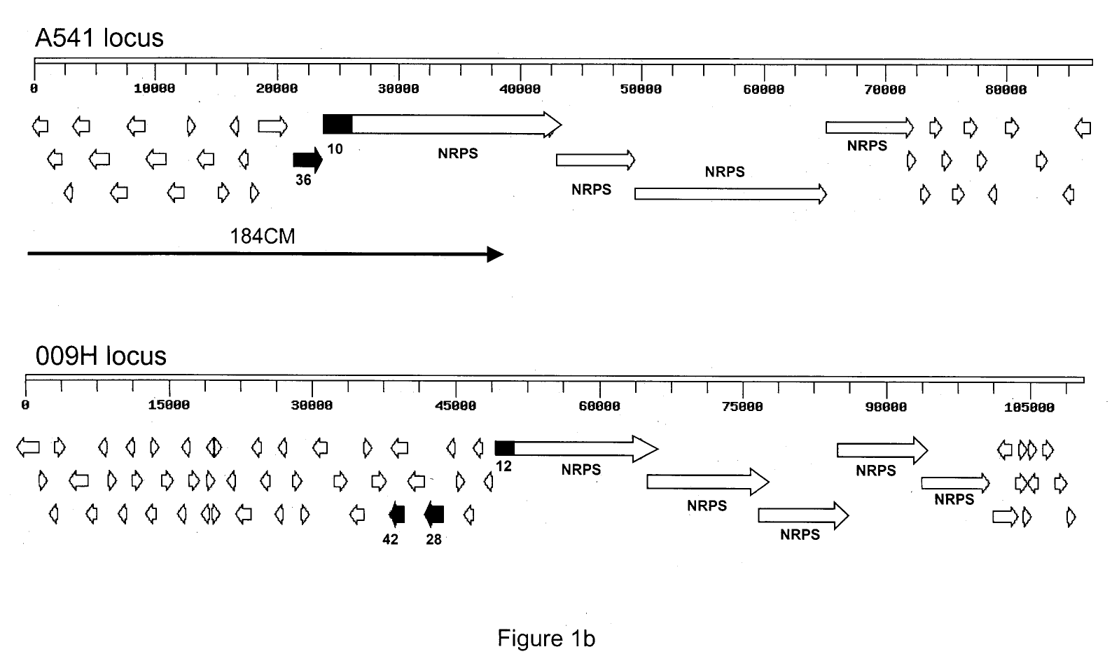

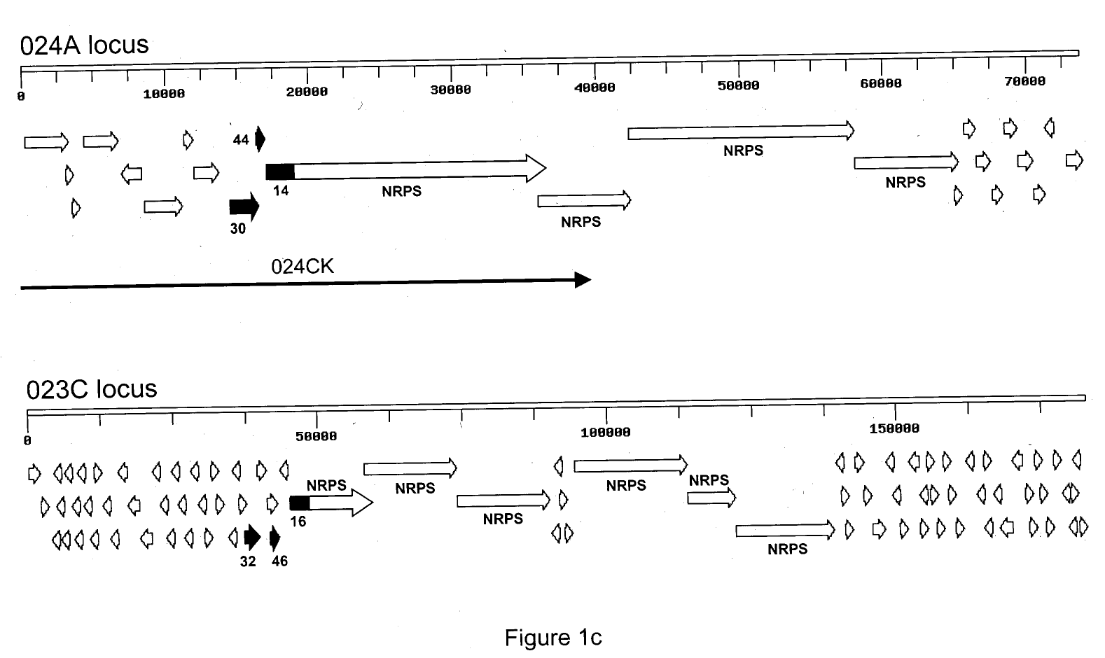

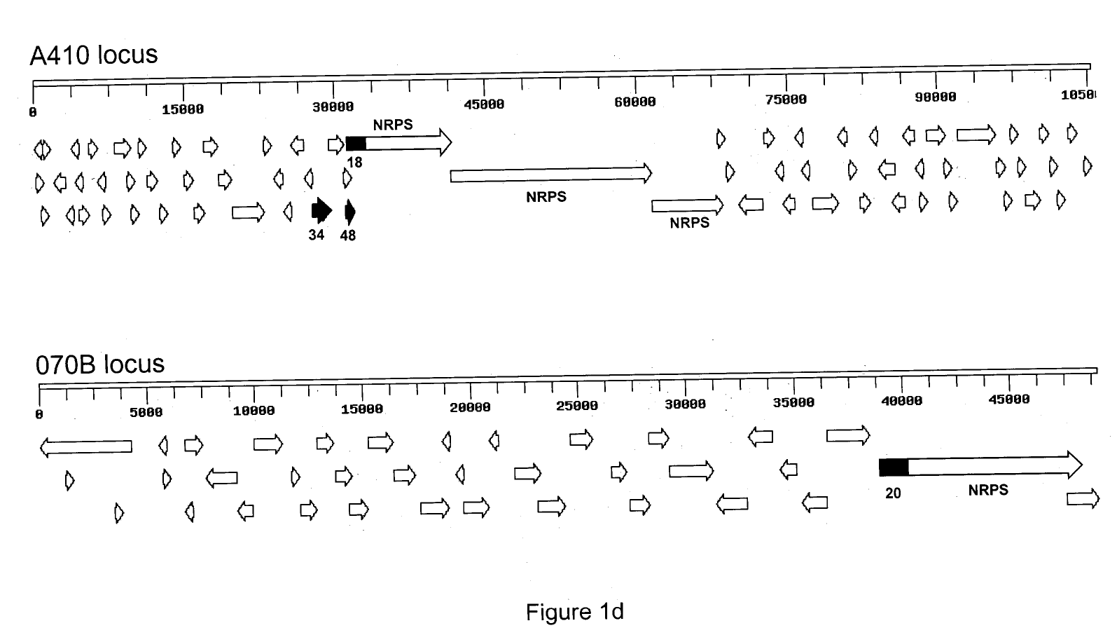

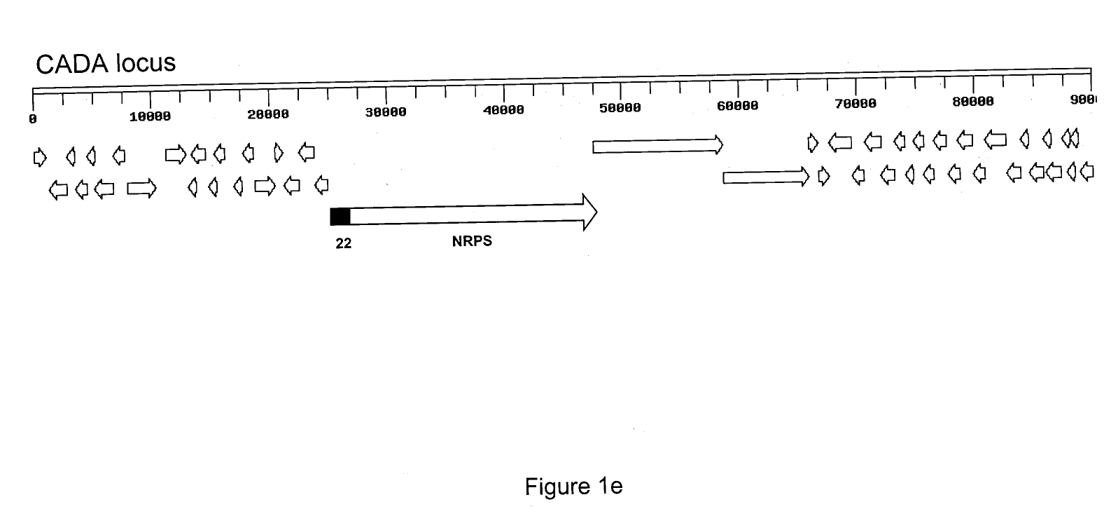

The method used to clone DAPT, a partial locus formed of seven complete and one partial open reading frames (ORFs) (FIG.. 1a), is disclosed in U.S. Ser. No. 60/342,133. Actinomycetes generally produce lipopeptides using NRPS proteins and a number of the ORFs discovered corresponded to NRPS proteins. Moreover, one of the NRPS ORFs discovered contained the partial NRPS sequences previously demonstrated to be part of the A21978C locus, thereby confirming the identify of DAPT. The module and domain organization analysis of ORFs designated 7 to 9 in U.S. Ser. No. 60/342,133 is consistent with that expected for biosynthesis of A21978C as described in detail in U.S. Ser. No. 60/342,133. The nature and order of the amino acid residues specified by ORFs 7 to 9 coincide with the exact chemical structure of A21978C (see Table 3 and FIG. 1 of U.S. Ser. No. 60/342,133). This analysis, as described in detail in USSN 60/342,133 demonstrate beyond doubt that DAPT is indeed the biosynthetic locus for A21978C from S. roseosporus.

A541: Streptomyces fradiae strain NRRL 18158 was known to produce the lipopeptide antibiotic complex A54145 of known structure. However the biosynthetic locus for A54145 in Streptomyces fradiae (A541) was not known. We cloned, sequenced and annotated A541, as disclosed in detail in U.S. Ser. No. 60/342,133, U.S. Ser. No. 60/372,789 and in co-pending U.S. Ser. No. 10/XXX,XXX filed concurrently with the present application and also claiming priority from U.S. Ser. No. 60/342,133 and U.S. Ser. No. 60/372,789. The contents of U.S. Ser. No. 10/XXX,XXX are incorporated herein in its entirety for all purposes.

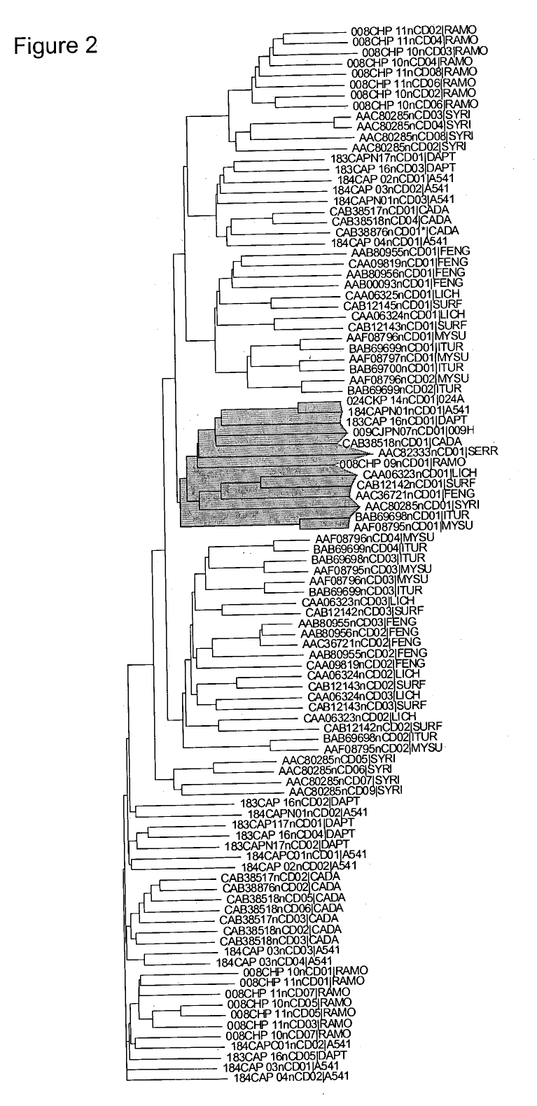

A541 contains three complete and one partial NRPS genes (FIG. 1b). Analysis of the NRPS ORFs revealed the presence of conserved domains involved in the recognition, activation, modification and condensation of amino acids. A total of 13 modules responsible for the condensation of 13 amino acid residues were identified as expected given that A54145 is composed of 13 amino acids. The adenylation domains were examined in order to determine the specificity of the amino acids that they activate and tether to the cognate thiolation domain of the NRPS. The nature and order of the amino acid residues specified by the NRPS ORFs exactly correspond to the nature and order of the amino acid residues found in the A54145 chemical structure (see Table 4 and FIG. 2 of U.S. Ser. No. 60/372,789). A methylation domain of ORF 8, module 5 as disclosed in U.S. Ser. No. 60/372,789 specifying the amino acid glycine corresponds to the amino acid incorporated in the fifth position of A54145 which is a N-methylated glycine (sarcosine). The nature and order of the amino acids specified by the NRPS genes as well as the presence of domains involved in the modification of some of the amino acids confirm that A541 is indeed the biosynthetic locus for A54145 in S. fradiae.

RAMO, DAPT and A541 were analyzed and compared. All three loci contain NRPS loading modules that begin with a condensation domain instead of the conventional adenylation-thiolation domains (FIGS. 1a and b, SEQ ID NOS: 6, 8 and 10 respectively). Such modules would generally be considered not to be capable of initiating peptide assembly on the assumption that the C-domain would likely interfere with this initiation process (see, for example, Linne and Marahiel, 2000, Biochemistry, Vol. 39, pp. 10439-10447). The nucleotide sequences of the members of the conserved family of unusual NRPS C-domains in RAMO, DAPT and A541 are disclosed as SEQ ID NOS: 5, 7 and 9 respectively. The polypeptides coding for the members of the conserved family of unusual NRPS C-domains in RAMO, DAPT and A541 are disclosed as SEQ ID NOS: 6, 8 and 10 respectively.

These C-domains were assessed by computer comparison with proteins found in the GenBank database of protein sequences (National Center for Biotechnology Information, National Library of Medicine, Bethesda, Md., USA) using the BLASTP algorithm (Altschul et al., supra) and the results are presented in Table 1. Amino acid sequence comparison analysis indicates that the RAMO, DAPT and A541 C-domains are related to condensation domains found in other lipopeptide-encoding NRPS systems.

The RAMO, DAPT and A541 C-domains were also compared to a collection of condensation domains from various lipopeptide NRPSs obtained from GenBank or disclosed herein. FIG. 2 shows the evolutionary relatedness of these C-domains. Apart from RAMO, DAPT, A541, FIG. 2 refers to additional lipopeptide biosynthetic loci by way of a four letter designations wherein CADA is the biosynthetic locus for the calcium-dependent antibiotic, FENG is the biosynthetic locus for fengycin, SURF is the biosynthetic locus for surfactin, SYRI is the biosynthetic locus for syringomycin, SERR is the biosynthetic locus for serrawettin, LICH is the biosynthetic locus for lichenysin, ITUR is the biosynthetic locus for iturin, and MYSU is the biosynthetic locus for mycosubtilin. All C-domains included in this analysis are full-length C domains. The convention used to identify and distinguish C domains in FIG. 2 is as follows. Those NRPS C-domain sequences that were obtained from the GenBank database are denoted by accessions beginning with three letters and are followed by digits (usually numbering 5). These first eight characters identifying each of the C domains correspond to the GenBank accession number. The lower case “n” serves to denote “NRPS domain”, and the “CD” followed by two digits denotes “C domain” and its number relative to the other C domains contained on that polypeptide sequence. For example “AAC80285nCD06|SYRI” represents the amino acid sequence corresponding to the sixth C domain contained on the GenBank entry AAC80285 for an NRPS from the syringomycin biosynthetic locus. The NRPS C domain sequences that are disclosed for the first time in this application, in U.S. provisional patent application Ser. No. 60/342,133 USSN or U.S. patent application Ser. No. 09/976,059 USSN follow a similar nomenclature (nCD00) but are denoted by nine-character accessions beginning with three numbers.

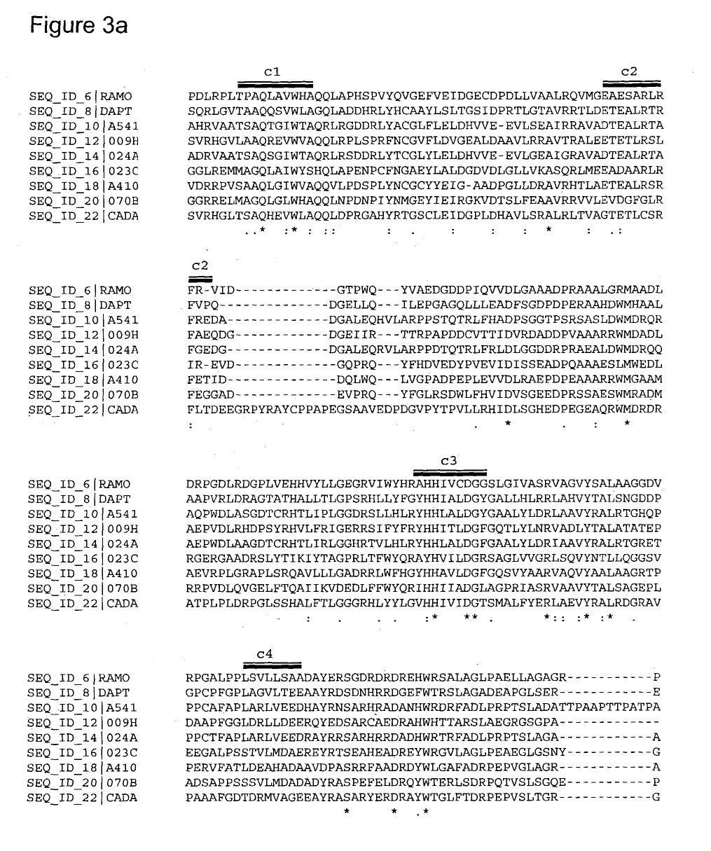

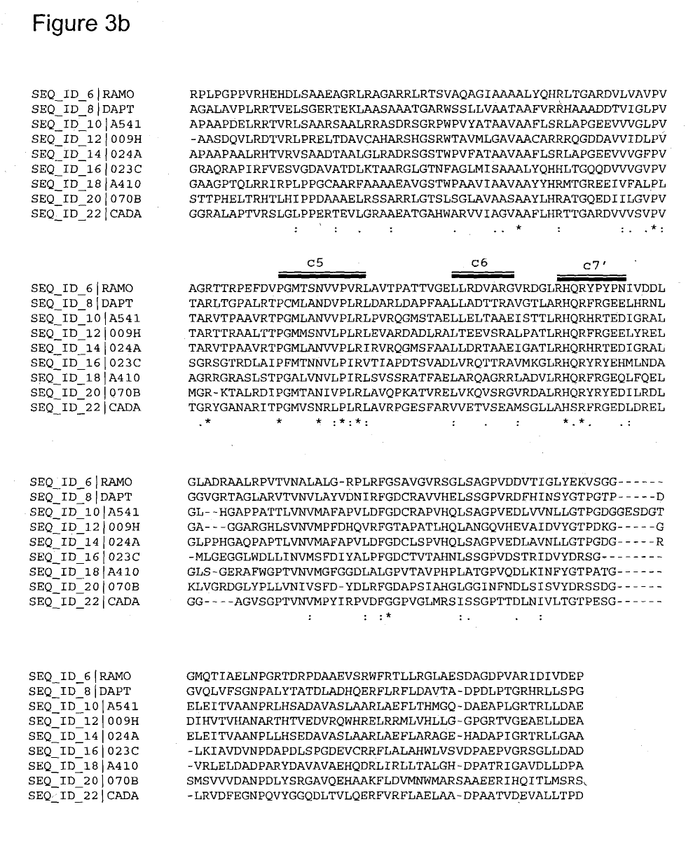

Analysis of a clustal alignment of the C-domains clearly shows that these domains are evolutionarily related to C-domains found in the starter modules of known N-acylated lipopeptides such as calcium-dependent antibiotic (CADA) (FIG. 1e, domain 22), surfactin (SURF), syringomycin (SYRI) and mycosubtilin (MYCO) among others (FIG. 2). Moreover, these special C-domains are significantly evolutionarily distant from regular condensation domains found in NRPSs that catalyze amide bond formation and condensation between two adjacent amino acids (FIG. 2). Alignment of these unusual C-domains demonstrates the conservation of motifs and specific amino acid residues important for their catalytic activity (FIG. 3). Based on these observations, the unusual C-domains are considered to catalyze N-acyl peptide linkages between a fatty acid and the amino terminal group of an amino acid.

A conserved family of activating enzymes (ADLE) was also found to be common to RAMO, DAPT and A541, although the gene encoding the activating enzyme in A541 was fused together with the gene encoding an acyl-carrier protein to form a single ORF (ADLF). The nucleotide sequences of the members of the conserved family of activating enzymes in RAMO, DAPT and A541 are disclosed as SEQ ID NOS: 23, 25 and 35 respectively. The polypeptides coding for these activating enzymes are disclosed as SEQ ID NOS: 24, 26 and 36 respectively. The ADLE activating enzyme portion of the ADLF fusion protein is referred to as SEQ ID NO: 36★.

A conserved family of acyl carrier proteins (ACPH) was also found to be common to RAMO, DAPT and A541, although the gene encoding the acyl carrier protein in A541 was fused together with the gene encoding the activating enzyme to form a single ORF (ADLF). The nucleotide sequences of the members of the conserved family of acyl carrier proteins in RAMO, DAPT and A541 are disclosed as SEQ ID NOS: 37, 39 and 35 respectively. The polypeptides coding for these acyl carrier proteins are disclosed as SEQ ID NOS: 38, 40 and 36 respectively. The ACPH acyl carrier portion of the ADLF fusion protein is referred to as SEQ ID NO: 36★★.

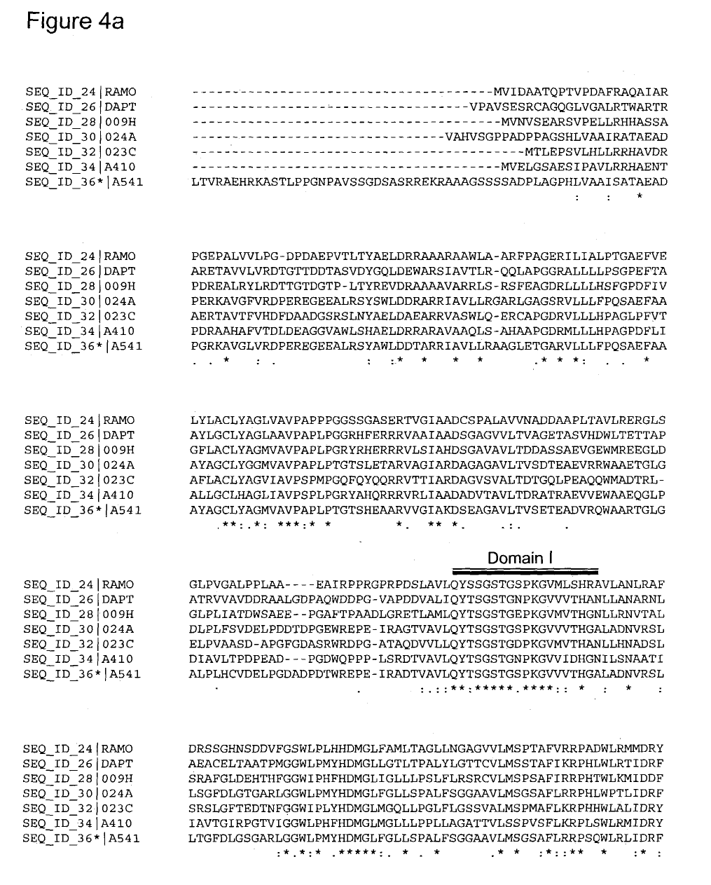

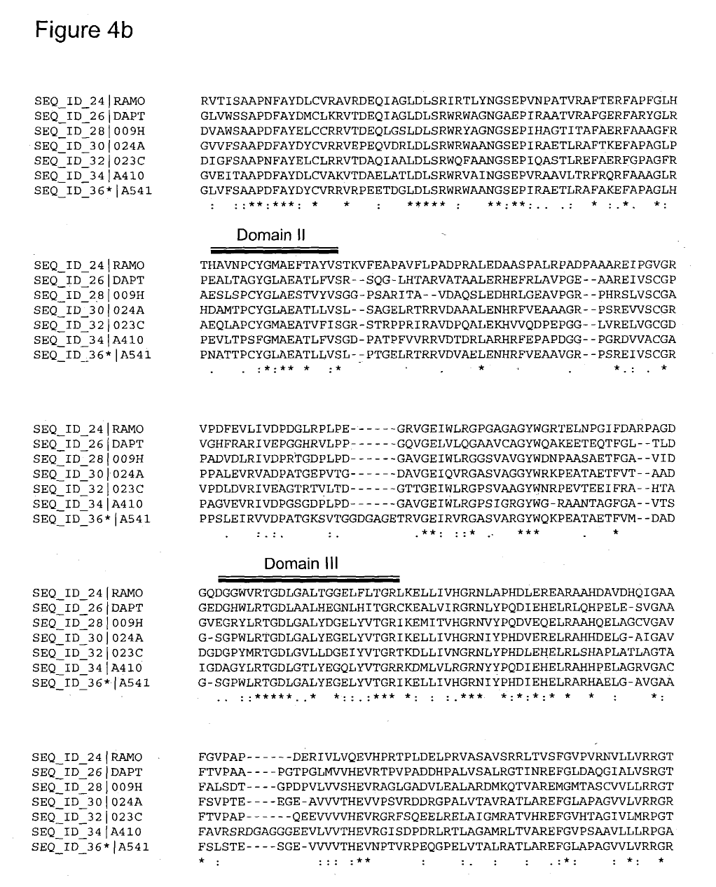

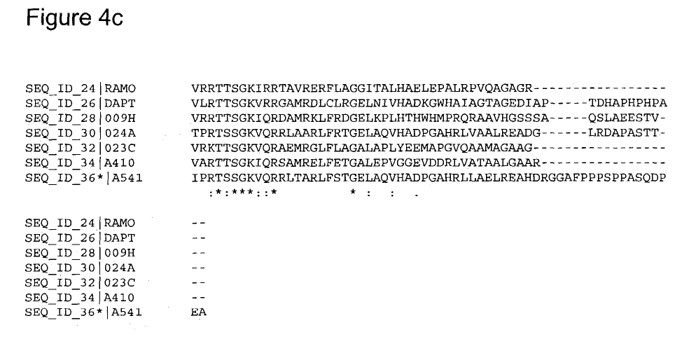

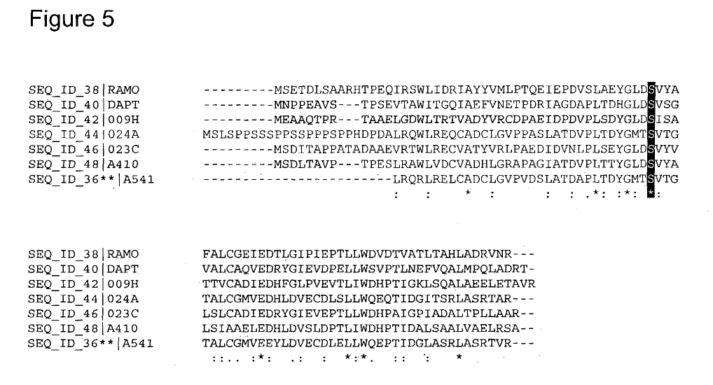

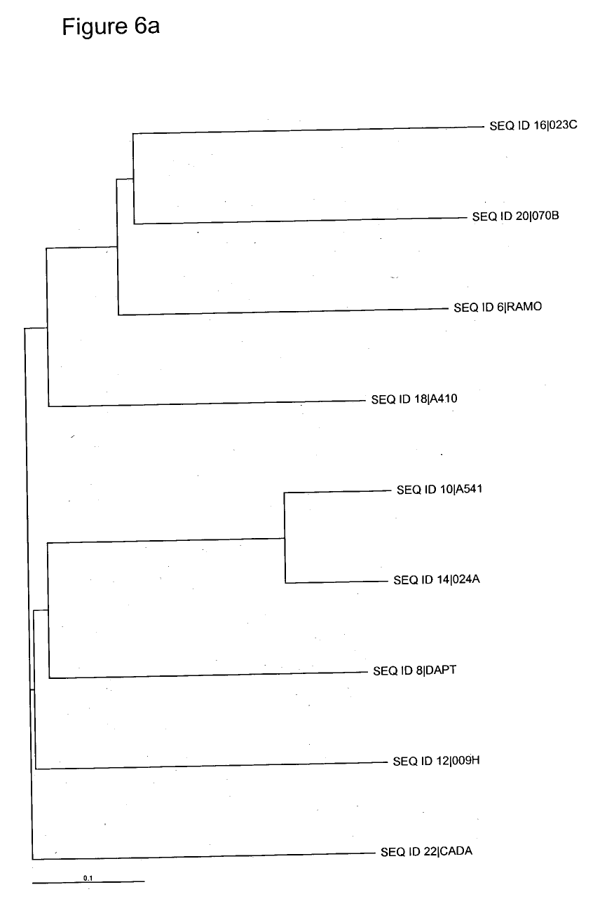

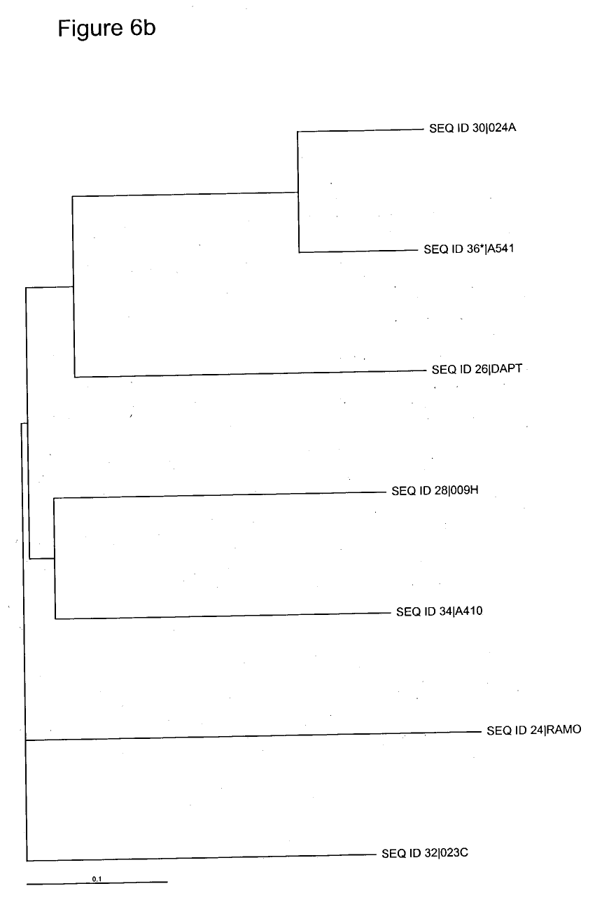

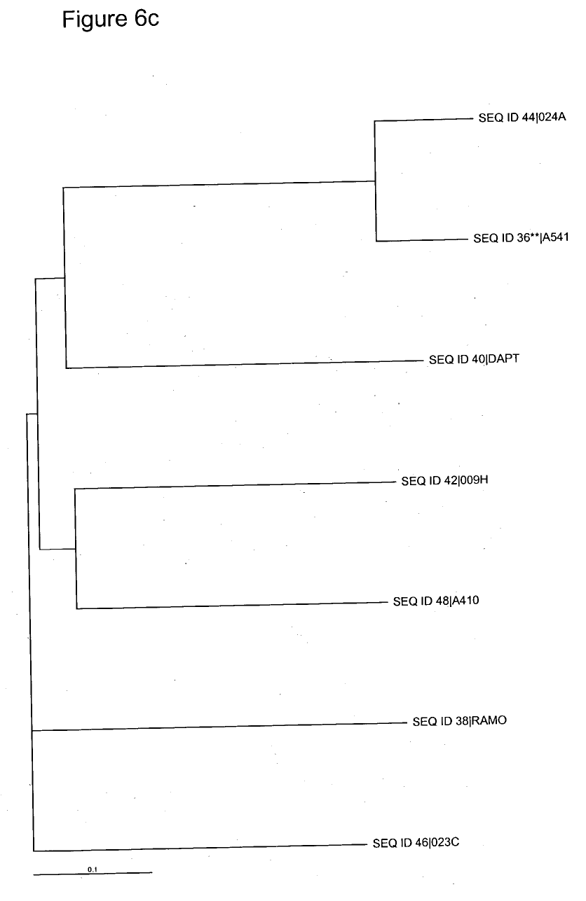

The biological function of the ADLE, ADLF and ACPH ORFs was assessed by amino acid sequence similarity analysis. The ADLE family of proteins shows similarity to various acyl CoA ligase enzymes whereas the ACPH family of proteins has sequence similarities to acyl carrier proteins found in the acyl-condensing polyketide synthase enzymatic systems (Tables 2 and 3). Clustal alignment of ADLE ORFs shows the conservation of domains and residues important for their enzymatic function (FIG. 4). Alignment of ACPH ORFs shows their overall sequence conservation and the absolute conservation of the serine residue that is modified by phosphopantetheinylation to form the active holo-acyl carrier protein (FIG. 5). Both ADLE and ACPH protein families are evolutionarily closely related to corresponding protein families from other lipopeptide loci (FIG. 6).

The ADLE and ACPH proteins as well as the acyl-specific C-domains of the invention are widely conserved throughout the biosynthetic loci of structurally diverse lipopeptides, including glycosylated lipopeptides and acidic glycopeptides. The only structural feature common to ramoplanin, A21978C and A54145 is a peptide backbone appended with a fatty acyl group at the N-terminal amino acid residue. Based on these correlations, the ADLE and ACPH proteins, and the unusual C-domain are considered to be responsible for activating and tethering fatty acyl groups and catalyzing the formation of the N-acyl peptide linkage.

### EXAMPLE 2

Biosynthesis of N-acylated Peptides:

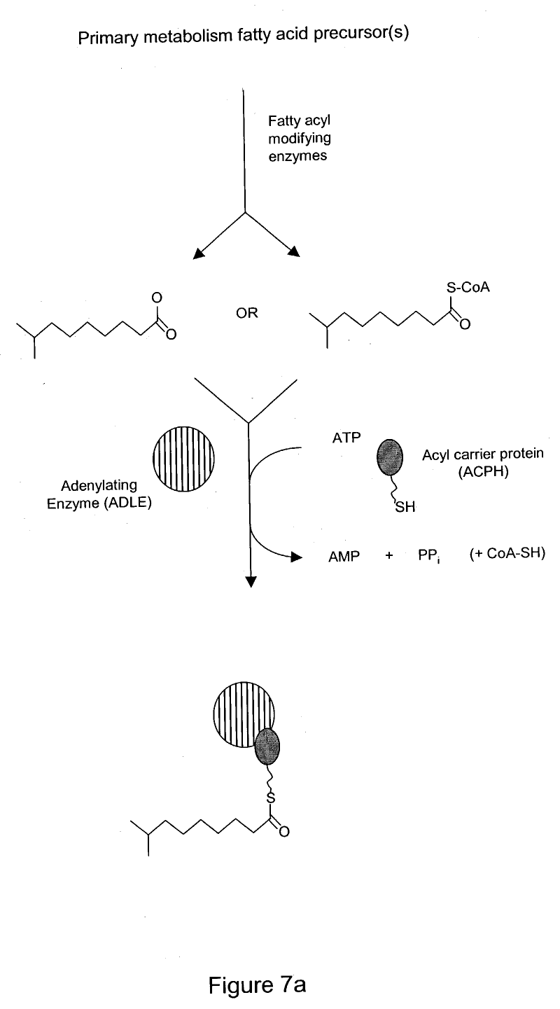

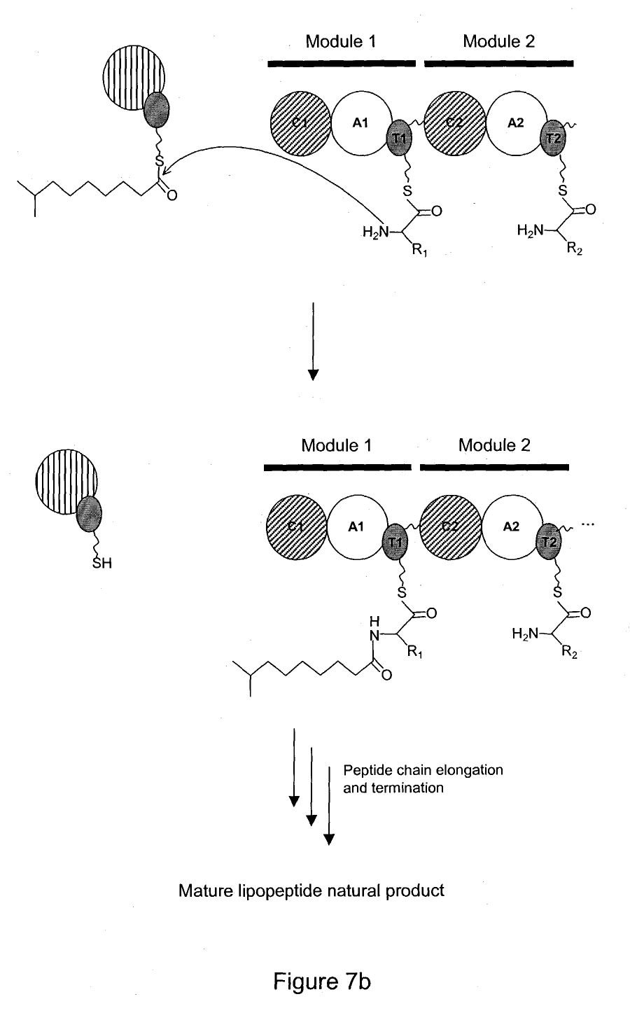

Despite the significant overall evolutionary distance between the lipopeptide-producing microorganisms described in this invention, they all contain closely related C-domains that are used for peptide N-acylation, a step which doubles as the peptide chain initiation step. Without intending to be limited to any particular biosynthetic scheme or mechanism of action, the ADLE, ACPH and unusual NRPS C-domain of the present invention can explain formation of the N-acyl peptide linkage found in lipopeptides. FIG. 7 illustrates a mechanism for NRPS chain initiation in which the fatty acyl group primes the synthesis of the peptide by the NRPS. CoA-linked fatty acyl precursors are channeled from the primary metabolic pool and modified while still attached to CoA by accessory enzymes such as oxidoreductases, epoxidases, desaturases, etc. encoded by genes of primary metabolism or by genes within the biosynthetic locus. The mature fatty acyl-CoA intermediate is then recognized by the cognate adenylating enzyme and transferred onto the phosphopantetheinyl prosthetic arm of the free holo-ACP, releasing CoA-SH and utilizing ATP in the process. It is alternatively contemplated that the adenylating enzyme may recognize free fatty acyl substrate(s) and transfer them onto the phosphopantetheinyl prosthetic arm of the free holo-ACP, utilizing ATP in the process. Once the fatty acyl group is tethered onto the free holo-ACP, the C domain of the first module carries out a reaction in which the carbonyl group of the activated fatty acyl is condensed with the amino group of the amino acid substrate that had been previously activated and tethered by the first module of the NRPS. Hence, peptide chain initiation and N-acylation are closely coupled. Subsequent peptide elongation and termination steps can then proceed as with typical NRPS modules.

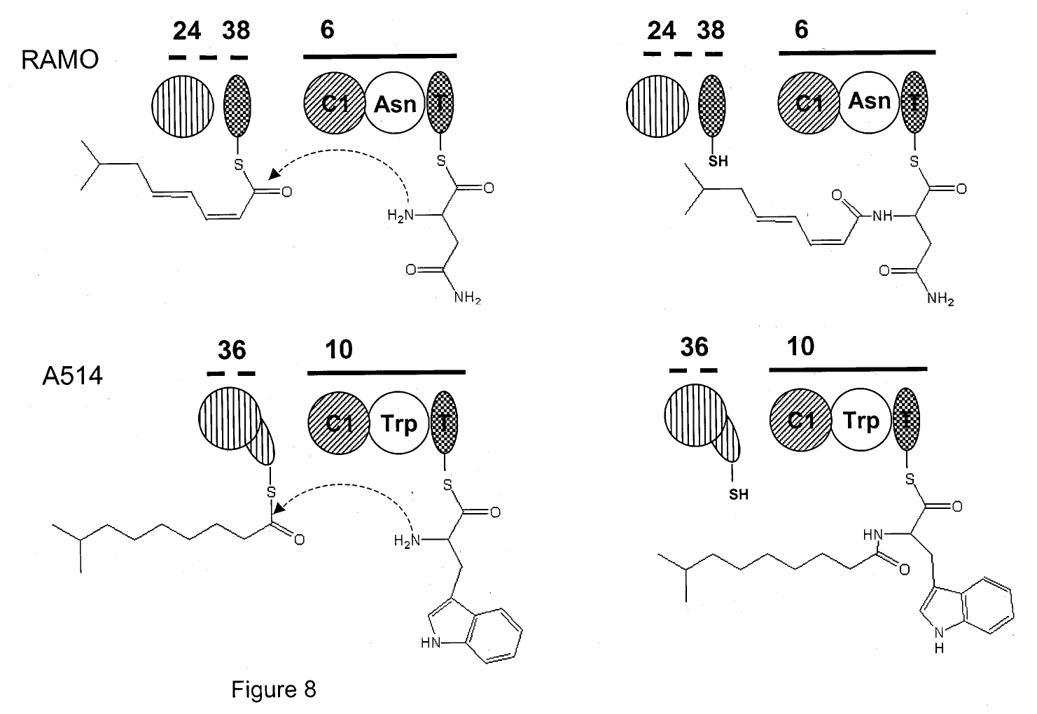

FIG. 8 illustrates the above-described amino acid N-acylation mechanism using specific examples in known lipopeptide biosynthetic pathways. In ramoplanin biosynthesis, an ADLE enzyme activates specific fatty acid moieties and subsequently tethers them onto the phosphopantetheinyl prosthetic arm of the ACPH (disclosed herein as SEQ ID NOS: 24 and 38 respectively). The carbonyl group of the activated fatty acyl is then condensed to the amino group of the asparagine residue (Asn) that had been previously activated by and tethered to the first module of the NRPS. The condensation reaction is catalyzed by the acyl-specific C-domain, disclosed herein as SEQ ID NO: 6, of the first module of the NRPS (FIGS. 1a and 8).

In another example, biosynthesis of the acylated peptide chain of antibiotic A54145 is initiated by activation and tethering of specific fatty acid units onto the ACPH component of the ADLF protein disclosed herein as SEQ ID NO: 36. ADLF represents the fusion of the two protein families, ADLE and ACPH, required for activation of fatty acids in lipopeptide biosynthesis. Once the fatty acid is activated, the acyl-specific C-domain of the first module, disclosed herein as SEQ ID NO 10, catalyzes the condensation of the carbonyl group of the fatty acyl and the amino group of the tryptophan residue (Trp) that had been previously activated by and tethered to the first module of the NRPS (FIGS. 1b and 8).

The same mechanism for peptide N-acylation may be present in other microorganisms. Evidence supporting this hypothesis includes the fact that other lipopeptide NRPS enzymes that have been identified in very diverse microorganisms contain a specialized C domain in the first module. Examples include the syringomycin biosynthetic locus from Pseudomonas syringae pv. syringae (Guenzi at al. (1998) J. Biol. Chem. Vol.273, pp. 32857-32863); the serrawettin W2 biosynthetic locus from Serratia liquefasciens MG1 (Lindum et al. (1998) Vol 180, pp. 6384-6388); the fengycin biosynthetic loci from Bacillus subtilis b213 and A1/3 (Steller et al. (1999) Chem. Biol. Vol. 6, pp. 31-41); the surfactin biosynthetic locus from Bacillus swotilis; the lichenysin biosynthetic locus from Bacillus licheniformis (Konz et al. (1999) J. Bact. Vol. 181, pp. 133-140); and the “calcium-dependent antibiotic” (CADA) biosynthetic locus from Streptomyces coelicolor A3(2) (Hajati et al. (2002) Chem. Biol. Vol. 9, pp. 1175-1187). The CADA biosynthetic locus does not apparently have an adenylating enzyme homologue but it does contain a free acyl carrier protein that may participate together with the unusual C domain of the first NRPS module in the N-acylation mechanism. Therefore, certain fatty acids may require specialized enzymes to transfer the fatty acyl moiety onto the acyl carrier protein, but once tethered onto the free acyl carrier protein the mechanism is analogous to that outlined in FIG. 7. It is noteworthy to point out that the fatty acyl moiety of CDA is unique in that it contains an epoxy modification. Hence such fatty acids may be transferred onto the ACP by some other specialized enzyme.

It is possible that the N-acylation mechanism of the present invention extends beyond bacteria to even more diverse microorganisms such as lower eukaryotes and other organisms. For example, the fungi Aspergillus nidulans var. roseus, Glarea lozoyensis, and Aspergillus japonicus var. aculeatus are known to produce the antifungal lipopeptides echinocandin B, pneumocandin B0, and aculeacin A, respectively (Hino et al. (2001) Journal of Industrial Microbiology and Biotechnology Vol 27, pp. 157-162). Based on the overall similarity between fungal and bacterial NRPS systems and on the fact that we have shown that very diverse NRPS systems employ the same mechanism of N-acylation, the mechanism of peptide N-acylation described in this invention is likely to be operative in these and/or other lipopeptide-producing lower eukaryotes as well.

Although the disclosed mechanism for peptide N-acylation is apparently widespread among very diverse microorganisms, it is not the only means by which lipopeptides can be generated. For example, the lipopeptides mycosubtilin and iturin A produced by Bacillus subtilis ATCC and RB14, respectively, are each assembled by multifunctional hybrid polypeptides comprising fused fatty acid synthase, amino transferase, and NRPS activities (Duitman et al. (1999) Proc. Natl. Acad. Sci USA. Vol. 96, pp. 13294-13299; Tsuge et al. (2001) J. Bact, Vol. 183, pp. 6265-6273). This alternative mechanism of peptide N-acylation may be more evolutionarily restricted as, to the best of our knowledge, it has been identified only in members of the genus Bacillus, and the lipopeptides produced by these biosynthetic loci are members of a distinct sub-group of lipopeptides that contain a P-amino fatty acyl moiety linked to the amino terminus of the peptide core. Despite the fact that this mechanism of N-acylation does not involve the action of ADLE and ACPH homologues, the C-domains that condense the β-amino fatty acyl moiety to the first amino acid of both mycosubtilin and iturin are found to cluster within the highlighted group of acyl-specific C-domains as shown in FIG. 2.

The widespread N-acylation mechanism for peptide natural products provides a knowledge-based approach for discovery and identification of lipopeptide biosynthetic loci in microorganisms. The highly conserved nucleotide sequences that are distinguishing signatures of the adenylating enzyme, the acyl carrier protein, and/or the specialized C-domain involved in the N-acylation mechanism can be identified and utilized as probes to screen libraries of microbial genomic DNA for the purpose of rapidly identifying, isolating, and characterizing lipopeptide biosynthetic loci in microorganisms of interest. The sequences of ADLE, ACPH proteins and the acyl-specific C-domain can also be used for in silico screening of large collections of microorganisms. Such a genetic-based screen has the added advantage over traditional fermentation approaches in that organisms having the genetic potential to produce lipopeptide natural products can be identified without the laborious fermentation, isolation, and characterization of the lipopeptide natural product. In addition, those organisms that normally produce lipopeptides only at very low or undetectable amounts or those organisms that only produce lipopeptides under very specialized growth conditions can nevertheless be readily identified using this genetic approach.

### EXAMPLE 3

Identification of Putative Lipopeptide Biosynthetic Locus 009H:

The sequences of the ADLE, ACPH and the acyl-specific C-domain were used in silico to screen a proprietary database of bacterial secondary metabolism loci, DECIPHER® (Ecopia BioSciences Inc; CA 2,352,451). To facilitate sequence comparisons, a protein domain database was generated that is part of the DECIPHER® database and comprises domains from multimodular proteins such as NRPSs and polyketide synthases, as well as equivalent domains found in non-modular proteins.

Protein sequences from loci RAMO, DAPT and A541 corresponding to acyl-specific C-domains, disclosed as SEQ ID NOS: 6, 8 and 10 respectively, ADLE ORFs, disclosed as SEQ ID NOS: 24, 26 and 36★, and ACPH ORFs, disclosed as SEQ ID NOS: 38, 40 and 36★★, were compared to the DECIPHER® domain database using the BLASTP algorithm (Altschul et al., supra). Moreover, consensus sequences from the acyl-specific C-domain, the ADLE and ACPH proteins, generated using the HMMER software package as described herein and disclosed as SEQ ID NOS: 1, 2, 3 and 4, were also compared to the DECIPHER® domain database.

Determination of sequence homology is assisted by the E value that indicates whether two sequences display sufficient similarity to justify an inference of homology. An E value of 0.00 indicates a perfect homolog. The E values are calculated as described in Altschul et al. 1990, J. Mol. Biol. 215(3): 403-410; in Altschul et al.1993, Nature Genetics 3: 226-272.

Comparison analysis of acyl-specific C-domain sequences with sequences from over 450 loci in the DECIPHER® database revealed the presence of a condensation domain, disclosed herein as SEQ ID NO: 12, that is included in locus 009H found in Streptomyces ghanaensis (NRRL B-12104). Table 4 shows that SEQ ID NO: 12 shows higher sequence similarity with sequences from the acyl-specific C-domains of RAMO, DAPT and A541 (that condense an acyl group to the amino terminal group of an amino acid) than with a typical NRPS condensation domain that catalyzes joining of two amino acids, as exemplified by the C-domain of the first module found in the ramoplanin ORF13 as described in detail in PCT/CA01/01462.

TABLE 4

| SEQ | | | Target Domain |
|---|---|---|---|
| ID NO | Probing Domain | E value | SEQ ID NO |
| 1 | Consensus C1 | 4 e−54 | 12 |
| 2 | Consensus C2 | 1 e−115 | 12 |
| 6 | RAMO C-domain | 4 e−44 | 12 |
| 8 | DAPT C-domain | 4 e−75 | 12 |
| 10 | A541 C-domain | 1 e−65 | 12 |
| — | RAMO ORF13, C-domain | 4 e−11 | 12 |
| 3 | Consensus ADLE | 0.00 | 28 |
| 24 | RAMO ADLE | 1 e−118 | 28 |
| 26 | DAPT ADLE | 1 e−141 | 28 |
| 36* | A541 ADLE | 1 e−141 | 28 |
| 4 | Consensus ACPH | 2 e−22 | 42 |
| 38 | RAMO ACPH | 4 e−15 | 42 |
| 40 | DAPT ACPH | 6 e−15 | 42 |
| 36** | A541 ACPH | 3 e−07 | 42 |

Similarly, ADLE domains with SEQ ID NOS: 3, 24, 26 and 36★ as well as ACPH domains with SEQ ID NOS: 4, 38, 40 and 36★★ were compared to the DECIPHER® domain database. Comparison analysis indicated the presence of proteins with high sequence homology to ADLE and ACPH sequences, disclosed as SEQ ID NOS: 28 and 42 respectively, also found in the 009H locus. The relatedness of SEQ ID NOS: 12, 28 and 42 to acyl-specific C-domains, ADLE and ACPH proteins was further confirmed by clustal sequence alignment showing the conservation of specific protein domains and by phylogenetic analysis (FIGS. 3-6).

Closer inspection of locus 009H shows the presence of 4 NRPS ORFs composed of 13 modules (FIG. 1b). The first NRPS ORF begins with the acyl-specific C-domain (SEQ ID NO: 12) instead of a typical adenylation domain. The ADLE and ACPH proteins (SEQ ID NOS: 28 and 42, respectively) are found in close proximity to the NRPS carrying the acyl-specific C-domain indicating that all three enzymes are part of the same biosynthetic locus. The simultaneous presence of these three enzymes along with the N-terminal location of the acyl-specific C-domain and the presence of a multienzymatic NRPS complex is consistent with the biosynthesis of an N-acylated lipopeptide, specified by locus 009H.

### EXAMPLE 4

Identification of Putative Lipopeptide Biosynthetic Locus 023C

In silico screening of the DECIPHER® database with consensus protein sequences and with sequences from loci RAMO, DAPT and A541 corresponding to acyl-specific C-domains, disclosed as SEQ ID NOS: 1, 2, 6, 8 and 10 respectively, further revealed the presence of an acyl-specific C-domain in locus 023C present in Streptomyces aizunensis NRRL B-11277. As shown in Table 5, sequence comparison analysis demonstrates that the 023C acyl-specific C-domain, disclosed herein as SEQ ID No: 16, is more closely related to the N-acyl capping C-domains from RAMO, DAPT and A541 than to typical NRPS condensation domains represented by the C-domain of the first module found in the ramoplanin ORF13 as described in detail in PCT/CA01/01462.

TABLE 5

| SEQ | | | Target Domain |
|---|---|---|---|
| ID NO | Probing Domain | E value | SEQ ID NO |
| 1 | Consensus C1 | 1 e−152 | 16 |
| 2 | Consensus C2 | 4 e−53 | 16 |
| 6 | RAMO C-domain | 6 e−82 | 16 |
| 8 | DAPT C-domain | 8 e−45 | 16 |
| 10 | A541 C-domain | 1 e−32 | 16 |
| — | RAMO ORF13, C-domain | 3 e−09 | 16 |
| 3 | Consensus ADLE | 0.00 | 32 |
| 24 | RAMO ADLE | 1 e−126 | 32 |
| 26 | DAPT ADLE | 1 e−146 | 32 |
| 36* | A541 ADLE | 1 e−134 | 32 |
| 4 | Consensus ACPH | 1 e−29 | 46 |
| 38 | RAMO ACPH | 2 e−16 | 46 |
| 40 | DAPT ACPH | 9 e−16 | 46 |
| 36** | A541 ACPH | 3 e−07 | 46 |

Proteins related to the ADLE and ACPH families of proteins, disclosed herein as SEQ ID 32 and 46, were also found in locus 023C (Table 5). The relatedness of SEQ ID NOS: 16, 32 and 46 to acyl-specific C-domains, ADLE and ACPH proteins was further confirmed by clustal alignment showing the conservation of specific protein domains and amino acid residues important for catalytic activity (FIGS. 3-5) and by phylogenetic analysis (FIG. 6).

Analysis of locus 023C shows the presence of 6 NRPS ORFs composed of 28 modules (FIG. 1c). The first NRPS ORF begins with the acyl-specific C-domain (SEQ ID NO: 16) indicative of the N-acyl capping mechanism (FIG. 7). Moreover, ADLE and ACPH proteins involved in fatty acid activation and tethering (SEQ ID NOS: 32 and 46 respectively) are also found in the 023 locus near the NRPS ORF, demonstrating that locus 023C is likely to encode an N-acylated lipopeptide metabolite.

### EXAMPLE 5

Identification of Putative Lipopeptide Biosynthetic Locus 024A:

Screening of the DECIPHER® database through protein homology analysis with sequences corresponding to acyl-specific C-domains (SEQ ID NOS: 1, 2, 6, 8 and 10) revealed the presence of an acyl-specific C-domain in locus 024A found in Streptomyces refuineus NRRL 3143. As shown in Table 6, BLASTP analysis demonstrates that the 024C encoded C-domain (SEQ ID NO: 14) is more closely related to domains condensing acyl groups to amino acids than to domains condensing two amino acids, as exemplified by the C-domain of the first module found in the ramoplanin ORF13 as described in detail in PCT/CA01/01462.

TABLE 6

| SEQ | | | Target Domain |
|---|---|---|---|
| ID NO | Probing Domain | E value | SEQ ID NO |
| 1 | Consensus C1 | 2 e−50 | 14 |
| 2 | Consensus C2 | 1 e−150 | 14 |
| 6 | RAMO C-domain | 3 e−43 | 14 |
| 8 | DAPT C-domain | 9 e−83 | 14 |
| 10 | A541 C-domain | 0.00 | 14 |
| — | RAMO ORF13, C-domain | 4 e−16 | 14 |
| 3 | Consensus ADLE | 0.00 | 30 |
| 24 | RAMO ADLE | 1 e−112 | 30 |
| 26 | DAPT ADLE | 1 e−155 | 30 |
| 36* | A541 ADLE | 0.00 | 30 |
| 4 | Consensus ACPH | 6 e−23 | 44 |
| 38 | RAMO ACPH | 4 e−12 | 44 |
| 40 | DAPT ACPH | 2 e−12 | 44 |
| 36** | A541 ACPH | 5 e−32 | 44 |

ADLE and ACPH related proteins, disclosed herein as SEQ ID NOS: 30 and 44, were also found in locus 024A (Table 6). Sequence alignments of all three proteins (SEQ ID NOS: 14, 30 and 44) show conservation of domains and amino acid residues important for catalytic activity of the corresponding enzymes (FIGS. 3-5). Additionally, these proteins are evolutionarily related to members of the acyl-specific C-domains, ADLE and ACPH families of proteins as indicated by phylogenetic analysis (FIG. 6).

Analysis of the 024A complete locus (FIG. 1c and U.S. Ser. No. 60/342,133, U.S. Ser. No. 30/372,789 and co-pending U.S. Ser. No. 10/XXX,XXX) reveals the presence of 4 NRPS ORFs composed of 13 modules. Consistent with an N-acyl peptide capping mechanism, the acyl-specific C-domain (SEQ ID NO: 14) is located at the N-terminal position of the first NRPS ORF, Moreover, the ADLE and ACPH ORFs (SEQ ID NOS: 30 and 44 respectively) are immediately adjacent to the acyl-specific C-domain suggesting a functional interaction between the three proteins. Based on these observations, locus 024A was predicted and subsequently proven to direct the biosynthesis of an N-acylated lipopeptide (see Example 8).

### EXAMPLE 6

Identification of Lipopeptide 41,012 Biosynthetic Locus A410:

Protein homology comparison of sequences specifying acyl-specific C-domains (SEQ ID NOS: 1, 2, 6, 8 and 10) with sequences found in the DECIPHER® database revealed the presence of a related C-domain, disclosed herein as SEQ ID 18, in locus A410 found in Actinoplanes nipponensis Routien ATCC 31145. This microorganism has been shown to synthesize an acidic polypeptide antibiotic of undetermined chemical strutcure, compound 41,012, that belongs to the amphomycin group of N-acylated lipopeptides (U.S. Pat. No. 4,001,397). As shown in Table 7, BLASTP demonstrates that the A410 encoded C-domain (SEQ ID NO: 18) is more closely related to domains condensing acyl groups to amino acids than to domains condensing two amino acids, as exemplified by the C-domain of the first module found in the ramoplanin ORF13 as described in detail in PCT/CA01/01462.

TABLE 7

| SEQ | | | Target Domain |
|---|---|---|---|
| ID NO | Probing Domain | E value | SEQ ID NO |
| 1 | Consensus C1 | 9 e−70 | 18 |
| 2 | Consensus C2 | 1 e−121 | 18 |
| 6 | RAMO C-domain | 3 e−64 | 18 |
| 8 | DAPT C-domain | 5 e−75 | 18 |
| 10 | A541 C-domain | 3 e−62 | 18 |
| — | RAMO ORF13, C-domain | 1 e−13 | 18 |
| 3 | Consensus ADLE | 0.00 | 34 |
| 24 | RAMO ADLE | 1 e−111 | 34 |
| 26 | DAPT ADLE | 1 e−137 | 34 |
| 36* | A541 ADLE | 1 e−141 | 34 |
| 4 | Consensus ACPH | 4 e−31 | 48 |
| 38 | RAMO ACPH | 1 e−14 | 48 |
| 40 | DAPT ACPH | 5 e−14 | 48 |
| 36** | A541 ACPH | 2 e−10 | 48 |

ADLE and ACPH related proteins, disclosed herein as SEQ ID NOS: 34 and 48, were also found in locus A410 (Table 7). Sequence alignments of all three proteins (SEQ ID NOS: 18, 34 and 48) show the conservation of domains and amino acid residues important for catalytic activity of these enzymes (FIGS. 3-5). Additionally, these proteins are evolutionarily related to members of the acyl-specific C-domains, ADLE and ACPH families of proteins as indicated by phylogenetic analysis (FIG. 6).

Locus A410 specifies 3 NRPS ORFs composed of 11 modules (FIG. 1d). Consistent with an N-acyl peptide capping mechanism, the acyl-specific C-domain (SEQ ID NO: 18) is located at the N-terminal position of the first NRPS ORF. Moreover, the ADLE and ACPH ORFs (SEQ ID NOS: 34 and 48 respectively) are found adjacent to the acyl-specific C-domain indicating that locus A410 specifies an N-acylated lipopeptide consistent with the described characteristics of antibiotic compound 41,012.

### EXAMPLE 7

Identification of Putative Lipopeptide Biosynthetic Locus 070B:

In silico screening of the DECIPHER® database with sequences corresponding to acyl-specific C-domains (SEQ ID NOS: 1, 2, 6, 8 and 10) revealed the presence of an acyl-specific C-domain in locus 070B found in Streptomyces sp. (Ecopia BioSciences, strain 070). As shown in Table 8, BLASTP analysis demonstrates that the 070B encoded C-domain (SEQ ID NO: 20) is more closely related to domains condensing acyl groups to amino acids than to domains condensing two amino acids, as exemplified by the C-domain of the first module found in the ramoplanin ORF13 as described in detail in PCT/CA01/01462.

TABLE 8

| SEQ | | | Target Domain |
|---|---|---|---|
| ID NO | Probing Domain | E value | SEQ ID NO |
| 1 | Consensus C1 | 1 e−153 | 20 |
| 2 | Consensus C2 | 2 e−67 | 20 |
| 6 | RAMO C-domain | 3 e−82 | 20 |
| 8 | DAPT C-domain | 3 e−47 | 20 |
| 10 | A541 C-domain | 6 e−48 | 20 |
| — | RAMO ORF13, C-domain | 9 e−16 | 20 |

Sequence alignment of the 070B acyl-specific C-domain (SEQ ID NO: 20) with related domains from various lipopeptide biosynthetic ORFs shows conservation of domains and amino acid residues important for catalytic activity of these enzymes (FIG. 3). Additionally, this protein is evolutionarily related to members of the acyl-specific C-domains as indicated by phylogenetic analysis (FIG. 6).

In contrast to the other loci presented herein, ADLE and ACPH related proteins were not detected in 070B.

Analysis of the 070B locus found in the DECIPHER® database shows the presence of an incomplete NRPS ORF composed of three modules (FIG. 1d). Consistent with the biosynthesis of an N-acylated lipopeptide, the acyl-specific C-domain is located at the N-terminus of the NRPS ORF. The lack of ADLE and ACPH sequences can be attributed to the fact that the sequence of the locus is not yet complete. Alternatively, 070B may be similar to the CADA locus in Streptomyces coelicolor A3(2) which specifies an N-acylated lipopeptide and lacks ADLE and ACPH related enzymes. Despite the potential absence of ADLE and ACPH in 070B, the presence and location of the acyl-specific C-domain clearly indicates that 070B specifies an N-acylated lipopeptide.

### EXAMPLE 8

Biosynthesis of an N-acylated Lipopeptide by Locus 024A:

Locus 024A in Streptomyces refuineus subsp. thermotolerans NRRL 3143 was shown to possess several characteristics of an N-acylated lipopeptide encoding locus, namely the presence of an acyl-specific C-domain (SEQ ID NO: 14) located at the N-terminus of the first NRPS ORF involved in the assembly of the polypeptide, ADLE and ACPH family proteins (SEQ ID NOS: 30 and 44 respectively) as well as an NRPS multienzymatic system composed of 13 modules (see Example 5 and FIG. 1c).

Protein homology analysis of the acyl-specific C-domain, the ADLE and the ACPH proteins with other proteins in the DECIPHER® database indicated a high homology of these proteins with corresponding proteins found in the A541 locus (SEQ ID NOS: 10, 36★ and 36★★) that specifies production of antibiotic A54145 in Streptomyces fradiae NRRL 18158 (Table 6 in example 5). Closer inspection of the two loci revealed the presence of an identical NRPS system that could be responsible for the synthesis of a 024A polypeptide scaffold identical to that of A54145 (FIGS. 1b and c and U.S. Ser. No. 60/342,133, U.S. Ser. No. 30/372,789 and co-pending U.S. Ser. No. 10/XXX,XXX).

Based on these observations and on the fact that there are known growth conditions for expressing lipopeptide A54145 in Streptomyces fradiae (U.S. Pat. No. 4,977,083), Streptomyces refuineus subsp. thermotolerans was grown under identical culture conditions to assess possible induction of locus 024A and determine the nature of the specified product.

Streptomyces fradiae and Streptomyces refuineus subsp. thermotolerans were grown at 30° C. for 48 hour in a rotary shaker in 25 mL of a seed medium consisting of glucose (10 g/L), potato starch (30 g/L), soy flour (20 g/L), Pharmamedia (20g/L), and CaCO₃ (2 g/L) in tap water. Five mL of this seed culture was used to inoculate 500 mL of production media in a 4L baffled flask. Production media consisted of glucose (25 g/L), soy grits (18.75 g/L), blackstrap molasses (3.75 g/L), casein (1.25 g/L), sodium acetate (8 g/L), and CaCO₃ (3.13 g/L) in tap water, and proceeded for 7 days at 30° C. on a rotary shaker. The production culture was centrifuged and filtered to remove mycelia and solid matter. The pH was adjusted to 6.4 and 46 mL of Diaion HP20 was added and stirred for 30 minutes. HP20 resin was collected by Buchner filtration and washed successively with 140 mL water and 90 mL 15% CH₃CN/H₂0, and the wash was discarded. HP20 resin was then eluted with 140 mL 50% CH₃CN/H₂0 (fraction HP20 E2). This pool was passed over a 5 mL Amberlite IRA68 column (acetate cycle) and the flow through (fraction IRA FT) was reserved for bioassay. The column was washed with 25 mL 50% CH₃CN/H₂0 and eluted with 25 mL 50% CH₃CN/H₂0 containing 0.1 N HOAc (fraction IRA E1), and then eluted with 25 mL 50% CH₃CN/H₂0 containing 1.0 N HOAc (fraction IRA E2). Biological activity was followed during purification by bioassay with Micrococcus luteus in Nutrient Agar containing 5 mM CaCl₂.

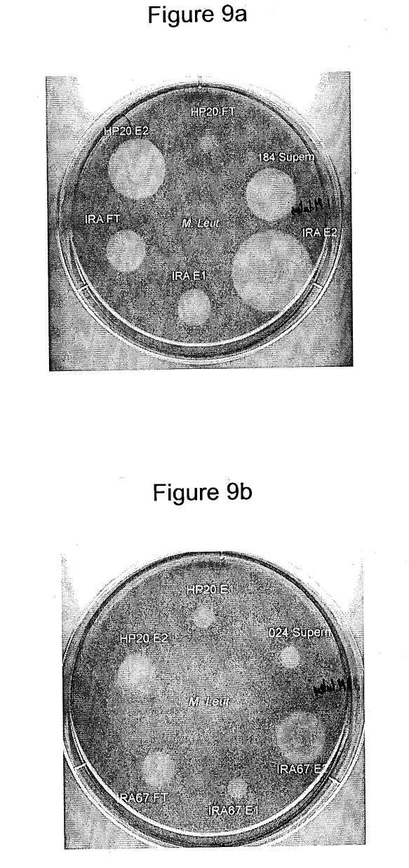

FIG. 9a is a photograph of a plate generated during extraction of an anionic lipopeptide from Streptomyces fradiae. FIG. 9a shows an enrichment of activity based on IRA67 anion exchange chromatography consistent with expression of an acidic lipopeptide. This activity is concentrated during the extraction procedure as indicated by the increased diameter of lysis rings. A54145 was detected via HPLC/MS in fraction IRA E2 as evidenced by mass ion ES²⁺=830.5 consistent with the structures of A54145C,D (U.S. Pat. No. 4,994,270).

FIG. 9b is a photograph of a plate generated during a similar extraction scheme performed on extracts from Streptomyces refuineus subsp. thermotolerans. FIG. 9b shows a similar enrichment of activity based on IRA67 anion exchange chromatography consistent with expression of an acidic lipopeptide. This activity is concentrated during the extraction procedure as indicated by the increased diameter of lysis rings. A mass ion of ES²⁺=830.5, identical to that of A54145, was present in fraction IRA E2 confirming that an N-acylated acidic lipopeptide, identical to A54145C,D, is produced by 024A in Streptomyces refuineus subsp. thermotolerans.

### EXAMPLE 9

Use of the N-acyl Capping Cassette to Engineer Peptide Synthetases Capable of Producing Novel Lipopeptides

The availability and understanding of lipopeptide N-acyl capping components increases the potential of redesigning (un)natural products by engineered peptide synthetases. It has been demonstrated that, using known molecular biology techniques, functional hybride peptide synthetases may be engineered that are capable of producing rationally designed peptide products (Mootz et al. (2000) Proc. Natl. Acad. Sci. U S A. Vol 97 pp. 5848-5853). Moreover, it has been postulated that through domain swapping, change-of-substrate specificity by mutagenesis, and an induced termination to achieve release of a defined shortened product, it may be possible to obtain a recombinant NRPS system that produces antipain, a potent cathepsin inhibitor produced by Streptomyces roseus and whose biosynthetic machinery is unknown (Doekel S, Marahiel MA. (2001) Metab. Eng. Vol 3 pp. 64-77). Mootz et al. (supra) described genetic engineering using an NRPS system to produce a peptide product that is not a naturally occurring product, and Doekel and Marahiel (supra) described a prophetic example of engineering an NRPS system to make the known natural product antipain.

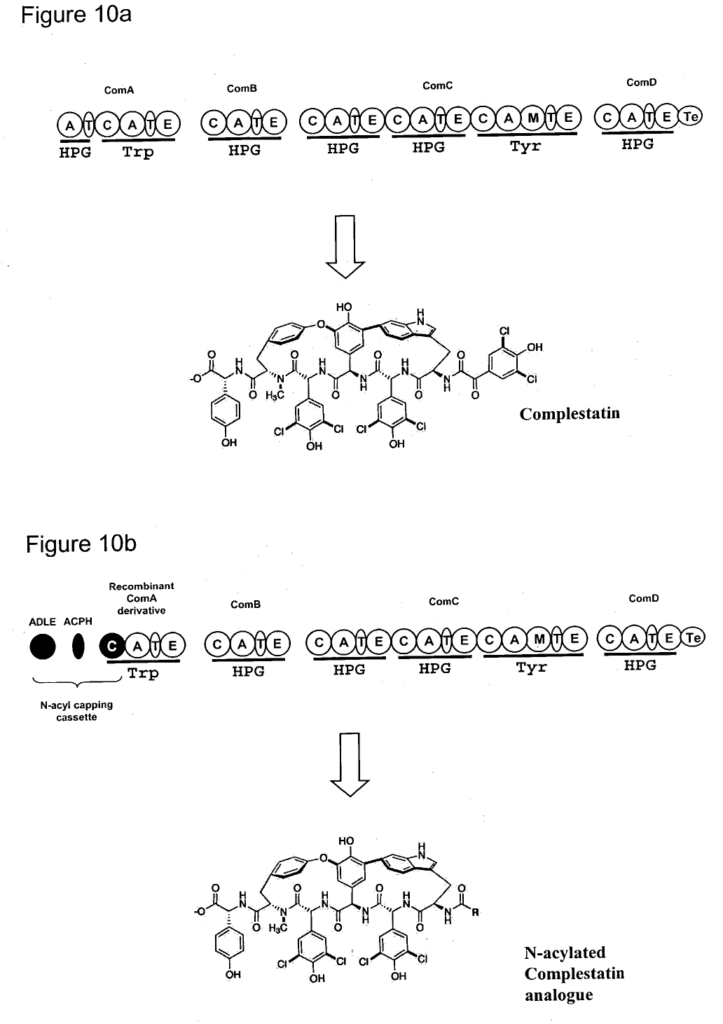

The following outlines a strategy whereby the NRPS biosynthetic machinery of a nonlipopeptide natural product, complestatin, can be modified so as to produce an N-acylated analogue of complestatin (FIG. 10).

Streptomyces lavendulae produces complestatin, a cyclic peptide natural product that antagonizes pharmacologically relevant protein-protein interactions including formation of the C4b, 2b complex in the complement cascade and gp120-CD4 binding in the HIV life cycle. Complestatin, a member of the vancomycin group of natural products, consists of an alpha-ketoacyl hexapeptide backbone modified by oxidative phenolic couplings and halogenations. The entire complestatin biosynthetic and regulatory gene cluster spanning ca. 50 kb was cloned and sequenced (Chiu et al. (2001) Proc. Natl. Acad. Sci. U S A Vol 98 pp. 8548-8553). It includes four NRPS genes, comA, comB, comC, and comD (FIG. 10, panel a). The comA gene encodes an NRPS that is composed of a loading module that incorporates hydroxyphenylglycine (HPG; or a derivative thereof) followed by a module that incorporates tryptophan (Trp), the first two residues of complestatin. Through domain swapping, the loading module and the C domain of the tryptophan-incorporating module can be replaced by one of the acyl-specific C-domains disclosed herein. Preferably, the acyl-specific C-domain of the A541, DAPT, or 024A loci would be used, as these domains are naturally specific for condensing an acyl moiety to a tryptophan residue. In addition to this domain swapping, the ADLE and ACPH genes would also be introduced into the system so as to provide a means to generate activated acyl substrates that can be used by the acyl-specific C domain. Thus, FIG. 10b depicts a rationally designed recombinant NRPS system that should give rise to N-acylated complestatin analogue(s). The recombinant NRPS system depicted in FIG. 10b could be employed either in vivo, using an appropriate recombinant host or in vitro using purified enzymes supplemented with the appropriate substrates.

One approach whereby N-acylated complestatin analogue(s) could be generated in vivo would involve the use of Streptomyces lavendulae, the complestatin producer, as the host strain. Briefly, the N-acyl capping cassette would replace the comA gene. This could be accomplished either by inactivation of the comA gene on the Streptomyces lavendulae chromosome followed by the introduction of a plasmid expressing the ADLE, ACPH, and the recombinant ComA derivative, or by physically replacing, by way of a double recombination (Keiser et al., supra) the comA gene on the Streptomyces lavendulae chromosome by a cassette containing genes encoding the ADLE, ACPH, and the recombinant ComA derivative. The resulting recombinant strains could be further modified to include genes involved in the biosynthesis of the acyl moieties and/or could be provided acyl moieties or precursors thereof in the fermentation medium.

One approach whereby N-acylated complestatin analogue(s) could be generated in vitro would involve the over-expression of the ADLE, ACPH, recombinant ComA, ComB, ComC, and ComD polypeptides in an appropriate host, for example E. coli, followed by the preparation of an extract or purified fraction thereof and use of said preparation together with appropriate substrates as outlined in Mootz et al. (2000). It is expected that, in the absence of accessory proteins the product produced by this in vitro system might not contain certain modifications such as the cross-linking of residues that is catalyzed by specific complestatin cytochrome P450 enzymes.

All patents, patent applications, and published references cited herein are hereby incorporated by reference in their entirety. While this invention has been particularly shown and described with reference to preferred embodiments thereof, it will be understood by those skilled in the art that various changes in form and details may be made therein without departing from the scope of the invention encompassed by the appended claims.

1. An isolated polynucleotide encoding an acyl-specific C-domain, wherein said isolated polynucleotide encodes a polypeptide which comprises at least 45% sequence identity to at least one sequence selected from SEQ ID NOS: 1 and 2.

2. An isolated polynucleotide comprising a sequence selected from the group consisting of:
(a) a sequence selected from the group consisting of SEQ ID NOS: 5, 7, 9, 11, 13, 15, 17 and 19;
(b) a sequence that is complementary to (a);
(c) a sequence which hybridizes to said sequence of (a) or (b) under conditions of high stringency; and
(d) a sequence which has at least 70% or higher homology to said sequence of (a), (b), or (c).

3. The isolated polynucleotide of claim 1, wherein said acyl-specific C-domain is involved in lipopeptide acyl-capping.

4. The isolated polynucleotide of claim 3, wherein said isolated polynucleotide resides in a gene locus selected from the group consisting of:
(a) the biosynthetic locus for ramoplanin from Actinoplanes sp. ATCC 33076;
(b) the biosynthetic locus for A21978C from Streptomyces roseosporus NRRL 11379;
(c) the biosynthetic locus for A54145 from Streptomyces fradiae ATCC 18158;
(d) the biosynthetic locus for the calcium-dependent antibiotic from Streptomyces coelicolor A3(2);
(e) the biosynthetic locus for a lipopeptide natural product from Streptomyces ghanaensis NRRL B-12104;
(f) the biosynthetic locus for a lipopeptide natural product from Streptomyces refuineus NRRL 3143;
(g) the biosynthetic locus for a lipopeptide natural product from Streptomyces aizunensis NRRL B-11277;
(h) the biosynthetic locus for a lipopeptide natural product from Actinoplanes nipponensis FD 24834 ATCC 31145; and
(i) the biosynthetic locus for a lipopeptide natural product from a Streptomyces sp. organism.

5. Two or more isolated polynucleotides, wherein the first polynucleotide is a polynucleotide of claim 1, and the second polynucleotide encodes a polypeptide selected from the group consisting of:
(j) a polypeptide having at least 55% sequence identity to SEQ ID NO: 3, and
(k) a polypeptide having at least 50% sequence identity to SEQ ID NO: 4.

6. An isolated polynucleotide comprising a sequence selected from the group consisting of:
(a) a sequence selected from the group consisting of SEQ ID NOs. 23, 25, 27, 29, 31, 33, 35, 37, 39, 41, 43, 45 and 47;
(b) a sequence that is complementary to (a);
(c) a sequence which hybridizes to said sequence of (a) or (b) under conditions of high stringency, and
(d) a sequence which has at least 70% or higher homology to said sequence of (a), (b), or (c).

7. The isolated polynucleotide of claim 6, wherein said isolated polynucleotide resides in a biosynthetic locus selected from the group consisting of:
(a) the biosynthetic locus for ramoplanin from Actinoplanes sp. ATCC 33076;
(b) the biosynthetic locus for A21978C from Streptomyces roseosporus NRRL 11379;
(c) the biosynthetic locus for A54145 from Streptomyces fradiae ATCC 18158;
(d) the biosynthetic locus for a lipopeptide natural product from Streptomyces ghanaensis NRRL B-12104;
(e) the biosynthetic locus for a lipopeptide natural product from Streptomyces refuineus NRRL 3143;
(f) the biosynthetic locus for a lipopeptide natural product from Streptomyces aizunensis NRRL B-11277;
(g) the biosynthetic locus for a lipopeptide natural product from Actinoplanes nipponensis FD 24834 ATCC 31145; and
(h) the biosynthetic locus for a lipopeptide natural product from a Streptomyces sp. organism.

8. An isolated acyl-specific C-domain, encoded by a polynucleotide which comprises a sequence selected from the group consisting of:
(a) a sequence selected from the group consisting of SEQ ID NOs. 5, 7, 9, 11, 13, 15, 17, 19; and
(b) a sequence that is complementary to (a);
(c) a sequence which hybridizes to said sequence of (a) or (b) under conditions of high stringency; and
(d) a sequence which has at least 70% or higher homology to said sequence of (a), (b), or (c).

9. An isolated acyl-specific C-domain comprising at least 45% sequence homology to at least one sequence selected from SEQ ID NO. 1 and SEQ ID NO. 2.

10. An isolated acyl-specific C-domain comprising a polypeptide sequence selected from the group consisting of:
(a) a sequence selected from the group consisting of SEQ ID NOs. 6, 8, 10, 12, 14, 16, 18, 20 and 22; and
(b) a sequence which has at least 70% or higher homology to said sequence of (a).

11. Two or more isolated polypeptides, wherein the first isolated polypeptide is an acyl-specific C-domain according to claim 9; and the second isolated polypeptide is selected from the group consisting of:
(a) a polypeptide having at least 55% identity to SEQ ID NO. 3 and
(b) a polypeptide having at least 50% identity to SEQ ID NO. 4.

12. An isolated polypeptide comprising a polypeptide selected from the group consisting of:
(a) SEQ ID NOs. 24, 26, 28, 30, 32, 34, 36, 38, 40, 42, 44, 46 and 48; and
(b) a sequence which has at least 70% or higher homology to said sequence of (a).

13. An N-acyl-capping cassette comprising at least one acyl-specific C-domain polypeptide and another polypeptide selected from the group consisting of an adenylating protein and an acyl-carrier protein.

14. A computer readable medium, comprising:
(a) a computer program stored on said media containing instructions sufficient to implement a process for effecting the identification, analysis, or modeling of a representation of a polynucleotide or polypeptide sequence;
(b) data stored on said media representing a sequence of a polynucleotide selected from the group consisting of:
i) a polynucleotide encoding an acyl-specific C-domain, said polynucleotide encoding a polypeptide having at least 45% sequence identity with either SEQ ID NO: 1 or SEQ ID NO: 2;
ii) a polynucleotide encoding a polypeptide having at least 55% sequence identity with SEQ ID NO: 3; and
iii) a polynucleotide encoding a polypeptide having at least 50% sequence identity with SEQ ID NO: 4; and
(c) a data structure reflecting the underlying organization and structure of said data to facilitate said computer program access to data elements corresponding to logical sub-components of the sequence, said data structure being inherent in said program and in the way in which said computer program organizes and accesses said data.

15. A computer readable medium, comprising:
(a) a computer program stored on said media containing instructions sufficient to implement a process for effecting the identification, analysis, or modeling of a representation of a polypeptide sequence;
(b) data stored on said media representing a sequence of a polypeptide selected from the group consisting of:
i) polypeptide representing an acyl-specific C-domain and having at least 45% sequence identity with either SEQ ID NO: 1 or SEQ ID NO: 2;
ii) a polypeptide having at least 55% sequence identity with SEQ ID NO: 3; and
iii) a polypeptide having at least 50% sequence identity with SEQ ID NO: 4 and
(c) a data structure reflecting the underlying organization and structure of said data to facilitate said computer program access to data elements corresponding to logical sub-components of the sequence, said data structure being inherent in said program and in the way in which said computer program organizes and accesses said data.

16. A memory for storing data that can be accessed by a computer programmed to implement a process for effecting the identification, analysis, or modeling of a sequence of a polynucleotide or a polypeptide, said memory comprising data representing a polynucleotide selected from the group consisting of:
(a) a polynucleotide encoding an acyl-specific C-domain, said polynucleotide encoding a polypeptide having at least 45% sequence identity with either SEQ ID NO: 1 or SEQ ID NO: 2;
(b) a polynucleotide encoding a polypeptide having at least 55% sequence identity with SEQ ID NO: 3; and
(c) a polynucleotide encoding a polypeptide having at least 50% sequence identity with SEQ ID NO: 4.

17. A memory for storing data that can be accessed by a computer programmed to implement a process for effecting the identification, analysis, or modeling of a sequence of a polypeptide, said memory comprising data representing a polypeptide selected from the group consisting of:
(a) a polypeptide having at least 45% sequence identity with either SEQ ID NO: 1 or SEQ ID NO: 2;
(b) a polypeptide having at least 55% sequence identity with SEQ ID NO: 3; and
(c) a polypeptide having at least 50% sequence identity with SEQ ID NO: 4.

18. A method for detecting a polypeptide involved in lipopeptide biosynthesis or a polynucleotide encoding such a polypeptide comprising the step of identifying:
(a) a polypeptide having at least 45% sequence identity to SEQ ID NO:1 or SEQ ID NO:2, or
(b) a polynucleotide encoding a polypeptide having at least 45% sequence identity to SEQ ID NO:1 or SEQ ID NO:2, and
wherein said at least 45% sequence identity indicates a polypeptide involved in lipopeptide biosynthesis.

19. A method according to claim 18 wherein the identifying step comprising the steps of:
(a) providing a reference polynucleotide or polypeptide sequence selected from the group consisting of polynucleotide or polypeptide sequences representing an acyl-specific domain;
(b) comparing said reference sequence to one or more candidate polynucleotide or polypeptide sequences stored on a computer readable medium;
(c) determining level of homology between said reference sequence and said one or more candidate sequences, and
(d) identifying a candidate sequence which shares at least 70% homology with reference sequence.

20. The method of claim 19, wherein said reference sequence is a polypeptide of SEQ ID NOS. 6, 8, 10, 12, 14, 16, 18, 20, 22 or a polynucleotide encoding a polypeptide of SEQ ID NOS. 6, 8, 10, 12, 14, 16, 18, 20 or 22.

21. The method of claim 19 further comprising determining structural motifs common to said candidate sequence and said reference sequence.

22. The method of claim 18 further comprising the step of identifying, in proximity to the polypeptide of a) or the polynucleotide of b) at least
c) one polypeptide having at least 55% sequence identity to SEQ ID NO: 3 or one polynucleotide sequence encoding a polypeptide having at least 55% sequence identity to SEQ ID NO: 3; or
d) one polypeptide having at least 50% sequence identity to SEQ ID NO: 4 or one polynucleotide sequence encoding a polypeptide having at least 50% sequence identity to SEQ ID NO: 4.

23. The method according to claim 22 wherein
(a) the polypeptide of c) or d) is a polypeptide of SEQ ID NO: 24, 26, 28, 30, 32, 34, 36, 38 or 40, or a polypeptide having at least 70% sequence identity to a polypeptide of SEQ ID NO: 24, 26, 28, 30, 32, 34, 36, 38 or 40; or
(b) the nucleotide of c) or d) is a nucleotide encoding a polypeptide of SEQ ID NO: 24, 26, 28, 30, 32, 34, 36, 38 or 40 or a nucleotide encoding a polypeptide having at least 70% sequence identity to a polypeptide of SEQ ID NO: 24, 26, 28, 30, 32, 34, 36, 38 or 40.

24. A computer system comprising:
(a) a database of reference sequences, wherein the reference sequences encode proteins involved in lipid biosynthesis, and wherein the reference sequences include one or more of:
(i) a polypeptide sequence representing an acyl-specific C-domain or a polynucleotide encoding an acyl-specific C-domain; and
(b) a user interface capable of:
(i) receiving a test sequence for comparing against each of the reference sequences in the database; and
(ii) displaying the results of the comparison.

25. A computer system of claim 24 wherein the reference sequences further include one or more of:
(iv) a polypeptide sequence representing an adenylating enzyme or a polynucleotide encoding an adenylating enzyme; and
(v) a polypeptide sequence representing an acyl carrier protein or a poynucleotide encoding an acyl carrier protein.

26. A computer system of claim 25 wherein
(a) the reference sequence of (i) is selected from SEQ ID NOS: 1, 2, 5, 6, 7, 8, 9, 10, 11, 12, 13, 14, 15, 16, 17, 18, 19, 20, 21 and 22;
(b) the reference sequence of (iv) is selected from SEQ ID NOS: 3, 23, 24, 25, 26, 27, 28, 29, 30, 31, 32, 33 and 34; and
(c) the reference sequence of (v) is selected from SEQ ID NO: 4, 37, 38, 39, 40, 41, 42, 43, 44, 45, 46, 47 and 48.
